(19) United States
(12) Patent Application Publication
(10) Pub. No.: US 2026/0327312 A1
(43) Pub. Date: Oct. 22, 2026

(54) COMPOSITIONS AND METHODS FOR TREATING CANCER WITH SUBCUTANEOUS ADMINISTRATION OF ANTI-PD1 ANTIBODIES

(71) Applicant: Merck Sharp & Dohme LLC, Rahway, NJ (US)

(72) Inventors: Omobolaji O. Akala, New York, NY (US); Carolina De Miranda Silva, Rahway, NJ (US); Elliot Keith Chartash, Basking Ridge, NJ (US); Mallika Lala, III, West New York, NJ (US); Kapil Mayawala, Iselin, NJ (US)

(73) Assignee: Merck Sharp & Dohme LLC, Rahway, NJ (US)

(21) Appl. No.: 19/534,095

(22) Filed: Feb. 9, 2026

Related U.S. Application Data
(63) Continuation of application No. 18/484,631, filed on Oct. 11, 2023.
(60) Provisional application No. 63/415,526, filed on Oct. 12, 2022.
(60) Provisional application No. 63/415,928, filed on Oct. 13, 2022.
(60) Provisional application No. 63/449,478, filed on Mar. 2, 2023.

(51) Int. Cl. C07K 16/28; A61K 9/19; A61K 31/282; A61K 31/337; A61K 31/519; A61K 31/573; A61K 31/714; A61K 38/47; A61K 39/00; A61K 39/395; A61P 35/00

(52) U.S. Cl. CPC C07K 16/2818; A61K 9/19; A61K 31/282; A61K 31/337; A61K 31/519; A61K 31/573; A61K 31/714; A61K 38/47; A61K 39/3955; A61P 35/00; C12Y 302/01035; A61K 2039/505; A61K 2039/545; C07K 2317/24

(57) ABSTRACT

The invention relates to compositions and methods for treating cancer in a patient comprising subcutaneously administering a PD-1 antagonist, e.g., an anti-PD-1 antibody (e.g. pembrolizumab), or antigen binding fragment thereof, with or without hyaluronidase every six weeks, in specific amounts to the patient. In specific embodiments, the amount of anti-PD-1 antibody, or antigen binding fragment thereof, is about 600 mg to about 1000 mg. In specific embodiments, the administration occurs about every three weeks, and the amount of anti-PD-1 antibody, or antigen binding fragment thereof, is about 300 mg to about 500 mg. In certain embodiments, the PD-1 antagonist is pembrolizumab, or an antigen binding fragment thereof. Also provided are compositions and kits formulated for subcutaneous administration comprising a particular dosage of an anti-PD-1 antibody, or antigen-binding fragment thereof, and uses thereof for treating cancer.

### CROSS-REFERENCE TO RELATED APPLICATIONS

This application is a continuation application of U.S. patent application Ser. No. 18/484,631, filed Oct. 11, 2023, which claims priority from and the benefit of U.S. Provisional Application No. 63/449,478, filed Mar. 2, 2023, U.S. Provisional Application No. 63/415,928, filed Oct. 13, 2022, and U.S. Provisional Application No. 63/415,526, filed Oct. 12, 2022; each of which is incorporated by reference in its entirety herein.

### REFERENCE TO SEQUENCE LISTING SUBMITTING ELECTRONICALLY

This application contains an electronic Sequence Listing which has been submitted in XML file format via Patent Center, the content of which is incorporated by reference herein in its entirety. The Sequence Listing XML file submitted via Patent Center is entitled “25591-US-CNT-2-SEQLIST.xml” with a size of 29,821 bytes; and a date of creation of Feb. 4, 2026.

### FIELD OF THE INVENTION

The invention relates to compositions and therapies useful for the treatment of cancer. In particular, the invention relates to a method for treating cancer which comprises administering to a patient in need thereof an anti-PD-1 antibody, or antigen binding fragment thereof, using the dosage regimens specified herein. Also provided are compositions and kits formulated for subcutaneous administration comprising a particular dosage of an anti-PD-1 antibody, or antigen-binding fragment thereof.

### BACKGROUND OF THE INVENTION

PD-1 is recognized as an important player in immune regulation and the maintenance of peripheral tolerance. PD-1 is moderately expressed on naive T, B and natural killer T (NKT) cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of cancers, e.g., ovarian, renal, colorectal, pancreatic, and liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33). Immune therapies targeting the PD-1 axis include monoclonal antibodies directed to the PD-1 receptor (KEYTRUDA™ (pembrolizumab), Merck and Co., Inc., Kenilworth, NJ, USA and OPDIVO™ (nivolumab), Bristol-Myers Squibb Company, Princeton, NJ, USA) and also those that bind to the PD-L1 ligand (MPDL3280A; TECENTRIQ™ (atezolizumab), Genentech, San Francisco, CA, USA; IMFINZI™ (durvalumab), AstraZeneca Pharmaceuticals LP, Wilmington, DE; and BAVENCIO™ (avelumab), Merck KGaA, Darmstadt, Germany). Both therapeutic approaches have demonstrated anti-tumor effects in numerous cancer types.

Hyaluronidases are enzymes that degrade hyaluronic acid present in the extracellular matrix. It is known that there are six types of hyaluronidases in humans: Hyal1, Hyal2, Hyal3, Hyal4, HyalPS1, and PH20/SPAM1. PH20/SPAM1 (hereinafter referred to as PH20) is expressed in the sperm plasma membrane and the acrosomal membrane.

Hyaluronidase hydrolyzes hyaluronic acid, thereby reducing the viscosity of hyaluronic acid in the extracellular matrix and increasing the permeability thereof into tissue (skin). The subcutaneous area of the skin has a neutral pH of about 7.0 to 7.5. Among the various types of hyaluronidases, PH20 is widely used (Bookbinder et al., 2006). In examples in which PH20 is used, PH20 is often co-administered with an antibody therapeutic agent which is injected subcutaneously (Bookbinder et al., 2006). rHuPH20, also known as Hylenex®, approved by the FDA is indicated as an adjuvant to increase the dispersion and absorption of other injected drugs.

Currently approved anti-PD-1 antibody treatments for use in multiple cancer indications are administered as an IV infusion at a dose of (i) either 200 mg or 2 mg/kg Q3W or (ii) 400 mg Q6W. It would be beneficial to develop a dosing schedule that allows for the administration of a safe and effective subcutaneous dose of an anti-PD-1 antibody with hyaluronidase that provides comparable exposure of the approved IV infusion dose. An alternative to IV infusions, such as a subcutaneous administration, would provide convenience and flexibility to patients, reduce patient time in the treatment room, and shorten the time needed by providers to administer the treatment.

### SUMMARY OF THE INVENTION

The invention provides alternative, convenient, cost-efficient, subcutaneous dosing regimens for treating a cancer patient with an anti-PD-1 antibody, or antigen-binding fragment thereof, wherein the dosing schedule is expected to provide a safe and effective dose of the anti-PD-1 antibody, or antigen-binding fragment thereof. Specifically, the invention provides a method of treating cancer in a human patient in need thereof comprising subcutaneously administering a dose of from about 600 mg to about 1000 mg of an anti-PD-1 antibody, or antigen binding fragment thereof, to the patient every six weeks; wherein the anti-PD-1 antibody or antigen-binding fragment thereof comprises (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively. In specific embodiments, the antibody or antigen binding fragment thereof is administered with a hyaluronidase including in specific embodiments a human hyaluronidase. In embodiments of the invention, the antibody or antigen-binding fragment is pembrolizumab or an antigen-binding fragment thereof. In a further embodiment, the anti-PD-1 antibody is pembrolizumab.

The invention also provides a method of treating cancer in a human patient in need thereof comprising subcutaneously administering to the patient approximately every three weeks a dose of from about 300 mg to about 500 mg of the anti-PD-1 antibody, or antigen binding fragment thereof, wherein the anti-PD-1 antibody or antigen-binding fragment thereof comprises (a) light chain (LC) complementarity determining regions (CDRs) LC-CDR1, LC-CDR2 and LC-CDR3 comprising a sequence of amino acids as set forth in SEQ ID NOs: 1, 2 and 3, respectively, and heavy chain (HC) CDRs HC-CDR1, HC-CDR2 and HC-CDR3 comprising a sequence of amino acids as set forth in SEQ ID NOs: 6, 7 and 8, respectively, and a hyaluronidase. In embodiments of the invention, the hyaluronidase is a human hyaluronidase. In embodiments of the invention, the antibody or antigen-binding fragment is pembrolizumab or an antigen-binding fragment thereof. In a further embodiment, the anti-PD-1 antibody is pembrolizumab.

The invention also relates to a pharmaceutical composition for subcutaneous injection comprising a dose of from about 600 mg to about 1000 mg of an anti-PD-1 antibody or antigen binding fragment thereof, and from about 700 Units to about 50000 Units of a human hyaluronidase. The invention also relates to a pharmaceutical composition for subcutaneous injection comprising a dose of from about 300 mg to about 500 mg of an anti-PD-1 antibody or antigen binding fragment thereof, and from about 700 Units to about 50000 Units of a human hyaluronidase.

In all of the disclosed treatment methods, compositions and uses herein, the anti-PD-1 antibody or antigen-binding fragment inhibits the binding of PD-L1 to PD-1, and preferably also inhibits the binding of PD-L2 to PD-1. In specific embodiments of the treatment methods, compositions and uses of the invention, the anti-PD-1 antibody or antigen-binding fragment is a monoclonal antibody, which specifically binds to PD-1 and blocks the binding of PD-L1 to PD-1. In one particular embodiment, the anti-PD-1 antibody comprises a heavy chain and a light chain, wherein the light and heavy chains comprise the amino acid sequences SEQ ID NO:5 and SEQ ID NO:10 or 11, respectively.

In specific embodiments of the disclosed treatment methods, compositions and uses, the cancer expresses one or both of PD-L1 and PD-L2. In specific embodiments, PD-L1 expression is present or elevated in the cancer.

### BRIEF DESCRIPTION OF THE DRAWINGS

FIG. 1 shows flat exposure-response for C_{trough} vs. tumor size change at 200 mg Q3W IV infusion in KEYNOTE-252. The C_{trough} at 400 mg Q6W IV infusion from KEYNOTE-555 fell within this range of C_{trough} where flat exposure-response is established.

FIG. 2A-F. Observed pharmacokinetic data from KEYNOTE-555 compared with the model-predicted pharmacokinetic profile for pembrolizumab 400 mg Q6W (A: cycle 1, B: steady state); the model-predicted C_{trough} for pembrolizumab 200 mg Q3W and 2 mg/kg Q3W compared to 400 mg Q6W (C: initial treatment six weeks, D: steady state); and the model predicted Cₘₐₓ for pembrolizumab 200 mg Q3W, 2 mg/kg Q3W, and 10 mg/kg Q2W compared to 400 mg Q6W (E: initial treatment six weeks, F: steady state).

FIG. 3A shows the distribution (5^{th}, 25^{th}, 50^{th}, 75^{th}, and 95^{th} percentiles; solid lines from bottom to top) of C_{trough} at cycle 1 using pharmacokinetics (“PK”) model-based simulations at a dose of 790 mg Q6W SC and 400 mg Q6W IV of pembrolizumab. Min IV refers to the 5th percentile value from distribution of the exposure measure for the 400 mg Q6W IV dose; Median IV refers to the 50th percentile value from distribution of the exposure measure for the 400 mg Q6W IV dose; Max IV refers to the 95th percentile value from distribution of the exposure measure for the 400 mg Q6W IV dose; Min IV, Median IV and Max IV are dashed lines from bottom to top.

FIG. 3B shows the distribution (5^{th}, 25^{th}, 50^{th}, 75^{th}, and 95^{th} percentiles; solid lines from bottom to top) of C_{trough} at steady state (cycle 3) using PK model-based simulations at a dose of 790 mg Q6W SC and 400 mg Q6W IV of pembrolizumab. Min IV refers to the 5th percentile value from distribution of the exposure measure for the 400 mg Q6W IV dose; Median IV refers to the 50th percentile value from distribution of the exposure measure for the 400 mg Q6W IV dose; Max IV refers to the 95th percentile value from distribution of the exposure measure for the 400 mg Q6W IV dose; Min IV, Median IV and Max IV are dashed lines from bottom to top.

FIG. 4A shows the distribution (5^{th}, 25^{th}, 50^{th}, 75^{th}, and 95^{th} percentiles solid lines from bottom to top) of AUC_{0-6 wks} at cycle 1 using PK model-based simulations at a dose of 790 mg Q6W SC and 400 mg Q6W IV of pembrolizumab. Min IV refers to the 5th percentile value from distribution of the exposure measure for the 400 mg Q6W IV dose; Median IV refers to the 50th percentile value from distribution of the exposure measure for the 400 mg Q6W IV dose; Max IV refers to the 95th percentile value from distribution of the exposure measure for the 400 mg Q6W IV dose; Min IV, Median IV and Max IV are dashed lines from bottom to top.

FIG. 4B shows the distribution (5^{th}, 25^{th}, 50^{th}, 75^{th}, and 95^{th} percentiles solid lines from bottom to top) of AUC_{0-6 wks} at steady state (cycle 3) using PK model-based simulations at a dose of 790 mg Q6W SC and 400 mg Q6W IV of pembrolizumab. Min IV refers to the 5th percentile value from distribution of the exposure measure for the 400 mg Q6W IV dose; Median IV refers to the 50th percentile value from distribution of the exposure measure for the 400 mg Q6W IV dose; Max IV refers to the 95th percentile value from distribution of the exposure measure for the 400 mg Q6W IV dose; Min IV, Median IV and Max IV are dashed lines from bottom to top.

FIG. 5 shows the distribution (5^{th}, 25^{th}, 50^{th}, 75^{th}, and 95^{th} percentiles; solid lines from bottom to top) of Cₘₐₓ at steady state (cycle 3) using PK model-based simulations at a dose of 790 mg Q6W SC and 400 mg Q6W IV of pembrolizumab. Min IV refers to the 5th percentile value from distribution of the exposure measure for the 400 mg Q6W IV dose; Median IV refers to the 50th percentile value from distribution of the exposure measure for the 400 mg Q6W IV dose; Max IV refers to the 95th percentile value from distribution of the exposure measure for the 400 mg Q6W IV dose; Min IV, Median IV and Max IV are dashed lines from bottom to top.

FIG. 6A shows the distribution (5^{th}, 25^{th}, 50^{th}, 75^{th}, and 95^{th} percentiles; solid lines from bottom to top) of C_{trough} at cycle 1 using PK model-based simulations at a dose of 395 mg Q3W (left figure is C_{trough} after 3 weeks; right figure is C_{trough} after 6 weeks) and 790 mg Q6W SC of pembrolizumab-HLN. Min Ref refers to the 5th percentile value from distribution of the exposure measure for the 790 mg Q6W SC dose; Median Ref refers to the 50th percentile value from distribution of the exposure measure for the 790 mg Q6W SC dose; Max Ref refers to the 95th percentile value from distribution of the exposure measure for the 790 mg Q6W SC dose; Min Ref, Median Ref and Max Ref are dashed lines from bottom to top.

FIG. 6B shows the distribution (5^{th}, 25^{th}, 50^{th}, 75^{th}, and 95^{th} percentiles; solid lines from bottom to top) of C_{trough} at steady state using PK model-based simulations at a dose of 395 mg Q3W and 790 mg Q6W SC of pembrolizumab-HLN. Min Ref refers to the 5th percentile value from distribution of the exposure measure for the 790 mg Q6W SC dose; Median Ref refers to the 50th percentile value from distribution of the exposure measure for the 790 mg Q6W SC dose; Max Ref refers to the 95th percentile value from distribution of the exposure measure for the 790 mg Q6W SC dose; Min Ref, Median Ref and Max Ref are dashed lines from bottom to top.

FIG. 7A shows the distribution (5^{th}, 25^{th}, 50^{th}, 75^{th}, and 95^{th} percentiles; solid lines from bottom to top) of AUC_{0-6 wks} at cycle 1 using PK model-based simulations at a dose of 395 mg Q3W and 790 mg Q6W SC of pembrolizumab-HLN. Min Ref refers to the 5th percentile value from distribution of the exposure measure for the 790 mg Q6W SC dose; Median Ref refers to the 50th percentile value from distribution of the exposure measure for the 790 mg Q6W SC dose; Max Ref refers to the 95th percentile value from distribution of the exposure measure for the 790 mg Q6W SC dose; Min Ref, Median Ref and Max Ref are dashed lines from bottom to top.

FIG. 7B shows the distribution (5^{th}, 25^{th}, 50^{th}, 75^{th}, and 95^{th} percentiles; solid lines from bottom to top) of AUC_{0-6 wks} at steady state using PK model-based simulations at a dose of 395 mg Q3W and 790 mg Q6W SC of pembrolizumab-HLN. Min Ref refers to the 5th percentile value from distribution of the exposure measure for the 790 mg Q6W SC dose; Median Ref refers to the 50th percentile value from distribution of the exposure measure for the 790 mg Q6W SC dose; Max Ref refers to the 95th percentile value from distribution of the exposure measure for the 790 mg Q6W SC dose; Min Ref, Median Ref and Max Ref are dashed lines from bottom to top.

FIG. 8 Similarity of pembrolizumab clearance based on a PK model across indications. Percentiles of the distribution of post-hoc estimated individual baseline clearance values among subjects per indication (number of subjects per indication shown above) are represented by the line (50th), box (25th-75th) and whiskers (5th-95th). The sample size (N) per group is provided above each box-whisker plot. NSCLC=non-small cell lung cancer; HN=head & neck squamous cell carcinoma; UC=urothelial cancer; MSIH=microsatellite instability high cancers; HCC=hepatocellular carcinoma; cHL=classical Hodgkin's lymphoma; PMBCL=primary mediastinal B-cell lymphoma.

### DETAILED DESCRIPTION OF THE INVENTION

The invention provides methods of treatment (e.g., methods of treating cancer) for a patient (e.g., a human patient) comprising subcutaneous administration of specified dosages of an anti-PD-1 antibody (e.g., pembrolizumab) or antigen-binding fragment thereof, and optionally a hyaluronidase (and in specific embodiments a human hyaluronidase). Such administration is expected to provide a safe and effective dose of the anti-PD-1 antibody or antigen-binding fragment thereof. Also provided are compositions and kits formulated for subcutaneous administration comprising a dosage of an anti-PD-1 antibody, or antigen-binding fragment thereof, and optionally a hyaluronidase (e.g., a human hyaluronidase), and uses thereof for treating cancer. In certain embodiments of the invention, the anti-PD-1 antibody is pembrolizumab or an antigen binding fragment of pembrolizumab.

### I. Abbreviations and Definitions

As used throughout the specification and appended claims, the following abbreviations apply:
AUC area under the concentration-time curve
AUCss area under the concentration-time curve at steady state
CDR complementarity determining region
Cl confidence interval
CL clearance
Cmax,ss peak concentrations at steady state
CPS combined positive score
CV coefficient of variation of between-subject distributions of parameters;
ECOG Eastern Cooperative Oncology Group
eGFR: estimated glomerular filtration rate
E-R exposure (concentration)-response
F bioavailability
FFPE formalin-fixed paraffin-embedded
FR framework region
GM geometric mean
HCC hepatocellular carcinoma
HNSCC head and neck squamous cell cancer
HL Hodgkin lymphoma
IgG immunoglobulin G
IHC immunohistochemistry or immunohistochemical
IMAX: maximum effect of time on CL
IV intravenous
ka first order absorption rate constant
LPS lymphoma proportion score
mAb monoclonal antibody
MCC Merkel cell carcinoma
MEL melanoma
MMR mismatch repair
MPS modified proportion score
MRI magnetic resonance imaging
MSI-H microsatellite instability-high
NCI National Cancer Institute
NSCLC non-small cell lung cancer
OS overall survival
PD-1 programmed death 1 (a.k.a. programmed cell death-1 and programmed death receptor 1)
PD-L1 programmed cell death 1 ligand 1
PD-L2 programmed cell death 1 ligand 2
PFS progression free survival
PK pharmacokinetic
Q intercompartmental clearance
Q2W one dose every two weeks
Q3W one dose every three weeks
Q6W one dose every six weeks
RCC renal cell carcinoma
RSE relative standard error
SC subcutaneous
TI₅₀ time at which 50% of maximum effect on clearance has been achieved
t_{lag} lag time for absorption
TPS tumor proportion score
Vc central volume of distribution
V_{H} immunoglobulin heavy chain variable region
V_{L} immunoglobulin light chain variable region
Vp peripheral volume of distribution

Presented population parameter estimates exclude effects of covariates; therefore, such estimates apply to a hypothetical typical patient with average characteristics.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,”, “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

The term “about” or “approximately”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” or “approximately” can mean a variation of ±0.1%, ±0.5%, ±1%, ±2%, ±3%, ±4%, ±5%, ±6%, ±7%, ±8%, ±9%, ±10% or ±11%.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” a cancer, as used herein, means to administer an anti-PD-1 antibody, or antigen-binding fragment, to a subject having a cancer, or diagnosed with a cancer, to achieve at least one positive therapeutic effect on the cancer, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating or abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (see, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In specific embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

Unless otherwise indicated, an “antibody fragment” or “antigen binding fragment” refers to antigen binding fragments of antibodies, i.e., antibody fragments that retain the ability to specifically bind to the antigen bound by the full-length antibody, e.g., fragments that retain one or more CDR regions, e.g., three heavy chain CDRs and three light chain CDRs. Examples of antigen binding fragments include, but are not limited to, Fab, Fab′, F(ab′)₂, and Fv fragments.

“Anti-PD-1 antibody” as used in any of the treatment methods, compositions and uses of the invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, compositions and uses of the invention in which a human individual is being treated, the anti-PD-1 antibody, or antigen binding fragment thereof, is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In specific embodiments the human constant region is selected from the group consisting of IgG1, IgG2, IgG3 and IgG4 constant regions, and in particular embodiments, the human constant region is an IgG1 or IgG4 constant region. In specific embodiments, the antigen binding fragment is selected from the group consisting of Fab, Fab′-SH, F(ab′)₂, scFv and Fv fragments.

“AUC” “and “Cmax” are pharmacokinetic measures of the systemic exposure to the drug (e.g. pembrolizumab) in humans after its administration, and are typically considered drivers of drug efficacy. “AUC” represents the average exposure over a dosing interval. “Cmax” is the maximum or highest (peak) drug concentration observed soon after its administration. In the specific case of pembrolizumab, which is administered as a subcutaneous injection, the peak concentration occurs immediately after end of infusion. Cmax is a metric that is typically considered a driver of safety.

“Biotherapeutic agent” means a biological molecule, such as an antibody or fusion protein, that blocks ligand/receptor signaling in any biological pathway that supports tumor maintenance and/or growth or suppresses the anti-tumor immune response.

The term “buffer” encompasses those agents which maintain the solution pH of the formulations of the invention in an acceptable range, or, for lyophilized formulations of the invention, provide an acceptable solution pH prior to lyophilization. The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

“C_{trough}” is the trough concentration achieved at the end of the dosing interval. The SC:IV C_{trough} ratio is the ratio (e.g. geometric mean ratio) of the C_{trough} achieved with the SC dose relative to an IV dose at the end of the same dosing interval.

“Co-administration” as used herein refers to the agents administered to a subject simultaneously or at about the same time. The agents may or may not be in physical combination prior to administration. For example, the anti-PD-1 antibody and the hyaluronidase can be contained in separate vials, and when in liquid solution, may be mixed into the same injection device, and administered simultaneously to the patient.

“Co-formulated” or “co-formulation” or “coformulation” or “co-formulated” as used herein refers to at least two different proteins or agents which are formulated together and stored as a combined product in a single vial, container, device or vessel (for example an injection device) rather than being formulated and stored individually and then mixed before administration or separately administered.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Additional cancers that may be treated in accordance with the invention include those characterized by elevated expression of one or both of PD-L1 and PD-L2 in tested tissue samples.

“CDR” or “CDRs” means complementarity determining region(s) in an immunoglobulin variable region, generally defined using the Kabat numbering system. “Chemotherapeutic agent” is a chemical compound useful in the treatment of cancer.

Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods, compositions, and uses of the invention include cytostatic and/or cytotoxic agents.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Diagnostic anti-PD-L monoclonal antibody” means a mAb which specifically binds to the mature form of the designated PD-L (PD-L1 or PD-L2) that is expressed on the surface of certain mammalian cells. A mature PD-L lacks the presecretory leader sequence, also referred to as leader peptide. The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 21)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO 2014/100079. These antibodies comprise the light chain and heavy chain variable region amino acid sequences shown in Table 2 below:

TABLE 2
Monoclonal Antibodies 20C3 and 22C3
20C3 Light Chain Mature Variable Region

| | |
|---|---|
| DIVMSQSPSSLAVSAGEKVTMSCKSSQSLLNSRTRKNYLAWY | SEQ ID NO: 22 |
| QQKPGQSPKLLIYWASTRESGVPDRFTGSGSGTDFTLTISSVQ | |
| AEDLAVYYCQQSYDVVTFGAGTKLELK | |

20C3 Heavy Chain Mature Variable Region

| | |
|---|---|
| QVQVQQSGAELAEPGASVKMSCKASGYIFTSYWMHWLKQR | SEQ ID NO: 23 |
| PGQGLEWIGYINPSSDYNEYSEKFMDKATLTADKASTTAYM | |
| QLISLTSEDSAVYYCARSGWLVHGDYYFDYWGQGTTLTVSS | |

22C3 Light Chain Mature Variable Region

| | |
|---|---|
| DIVMSQSPSSLAVSAGEKVTMTCKSSQSLLHTSTRKNYLAWY | SEQ ID NO: 24 |
| QQKPGQSPKLLIYWASTRESGVPDRFTGSGSGTDFTLTISSVQ | |
| AEDLAVYYCKQSYDVVTFGAGTKLELK | |

22C3 Heavy Chain Mature Variable Region

| | |
|---|---|
| QVHLQQSGAELAKPGASVKMSCKASGYTFTSYWIHWIKQRP | SEQ ID NO: 25 |
| GQGLEWIGYINPSSGYHEYNQKFIDKATLTADRSSSTAYMHL | |
| TSLTSEDSAVYYCARSGWLIHGDYYFDFWGQGTTLTVSS | |

Another anti-human PD-L1 mAb that has been reported to be useful for TIC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P. R. China; Catalog number 10084-R015).

“Framework region” or “FR” as used herein means the immunoglobulin variable regions excluding the CDR regions.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Hypervariable region” refers to the amino acid residues of an antibody that are responsible for antigen-binding. The hypervariable region comprises amino acid residues from a “complementarity determining region” or “CDR” (i.e., LC-CDR1, LC-CDR2 and LC-CDR3 in the light chain variable domain and HC-CDR1, HC-CDR2 and HC-CDR3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues refers to those variable domain residues other than the hypervariable region residues defined herein as CDR residues.

“Immunogenic agent” refers to a composition capable of inducing a humoral and/or cell-mediated immune response. Immunogenic agents may include, for example, attenuated cancerous cells, tumor antigens, antigen presenting cells such as dendritic cells pulsed with tumor derived antigen or nucleic acids, immune stimulating cytokines (e.g., IL-2, IFNα2, GM-CSF), and cells transfected with genes encoding immune stimulating cytokines, such as but not limited to GM-CSF.

“In need thereof” refers to in need of treatment.

“Kabat,” as used herein, means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers: BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-β-RII and ACTC can be used. Commercially available kits for MSI analysis include, for example, the Promega MSI multiplex PCR assay, FoundationOne® CDx (F1CDx) next generation sequencing based in vitro diagnostic device using DNA isolated from formalin-fixed, paraffin-embedded (FFPE) tumor tissue specimens.

“High frequency microsatellite instability” or “microsatellite instability-high (MSI-H)” refers to a tumor in which two or more of the five NCI markers indicated above show instability in its DNA or ≥30-40% of the total markers in its DNA demonstrate instability (i.e., have insertion/deletion mutations).

“Non-MSI-H cancer” as used herein refers to microsatellite stable (MSS) and low frequency MSI (MSI-L) cancer.

“Microsatellite Stable (MSS)” refers to a tumor in which none of the five NCI markers indicated above show instability in its DNA (i.e., have insertion/deletion mutations).

“Patient” (alternatively referred to as “subject” or “individual” herein) refers to a mammal (e.g., rat, mouse, dog, cat, rabbit) capable of being treated with the methods and compositions of the invention, most preferably a human. In specific embodiments, the patient is an adult patient. In other embodiments, the patient is a pediatric patient.

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include reverse transcription polymerase chain reaction (RT-PCR) and real-time quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if at least 1%, and preferably 5% of total tumor cells exhibit histologic evidence of cell-surface membrane staining.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD-L1 antibody may also be scored for PD-L1 protein expression by assessing PD-L1 expression in both the tumor cells and infiltrating immune cells in the tissue section using a scoring process. See WO 2014/165422. One PD-L1 scoring process comprises examining each tumor nest in the tissue section for staining, and assigning to the tissue section one or both of a modified H score (MHS) and a modified proportion score (MPS). To assign the MHS, four separate percentages are estimated across all of the viable tumor cells and stained mononuclear inflammatory cells in all of the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate or strong staining percentages. The estimated percentages, the sum of which is 100%, are then inputted into the formula of 1×(percent of weak staining cells)+2×(percent of moderate staining cells)+3×(percent of strong staining cells), and the result is assigned to the tissue section as the MHS. The MPS is assigned by estimating, across all of the viable tumor cells and stained mononuclear inflammatory cells in all of the examined tumor nests, the percentage of cells that have at least partial membrane staining of any intensity, and the resulting percentage is assigned to the tissue section as the MPS. In specific embodiments, the tumor is designated as positive for PD-L1 expression if the MHS or the MPS is positive.

Another method for scoring/quantifying PD-L1 expression in a tumor is the “combined positive score” or “CPS,” which refers to an algorithm for determining a PD-L1 expression score from a tumor sample of a patient. The CPS is useful in selecting patients for treatment with particular treatment regimens including methods of treatment comprising administration of an anti-PD-1 antibody in which expression of PD-L1 is associated with a higher response rate in a particular patient population relative to same patient population that does not express PD-L1. The CPS is determined by determining the number of viable PD-L1 positive tumor cells, the number of viable PD-L1 negative tumor cells, and the number of viable PD-L1 positive mononuclear inflammatory cells (MIC) in a tumor tissue from a patient having a tumor and calculating the CPS using the following formula:

$$\frac{( \# PD - L 1 positive tumor cells ) + ( \# PD - L 1 positive MIC )}{\begin{matrix}( \# PD - L 1 positive tumor cells ) + \\ ( \# PD - L 1 negative tumor cells )\end{matrix}} \times 100 \% .$$

In particular embodiments, the PD-L1 expression scoring method used is the “lymphoma proportion score.” Lymphoma is characterized by a homogeneous population of confluent cells which efface the architecture of the lymph node or the architecture of metastatic site. The “LPS” or “lymphoma proportion score” is the percentage of this population of cells which express PD-L1. When determining the LPS, no attempt is made to distinguish the truly neoplastic cells from the reactive cells. PD-L1 expression is characterized by partial or complete membrane staining at any intensity.

Yet another scoring method for PD-L1 expression is the “TPS” or “tumor proportion score,” which is the percentage of tumor cells expressing PD-L1 on the cell membrane. TPS typically includes the percentage of neoplastic cells expressing PD-L1 at any intensity (weak, moderate, or strong), which can be determined using an immunohistochemical assay using a diagnostic anti-human PD-L1 mAb, e.g., antibody 20C3 and antibody 22C3, described, supra. Cells are considered to express PD-L1 if membrane staining is present, including cells with partial membrane staining.

The level of PD-L mRNA expression may be compared to the mRNA expression levels of one or more reference genes that are frequently used in quantitative RT-PCR, such as ubiquitin C.

In specific embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In specific embodiments, PD-L1 expression in a tumor sample is determined to be elevated if PD-L1 protein (and/or PD-L1 mRNA) in the sample is at least 10%, 20%, or 30% greater than in the control.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 3. Pembrolizumab has been approved by the U.S. FDA as described in the Prescribing Information for KEYTRUDA™ (Merck & Co., Inc., Whitehouse Station, NJ USA; initial U.S. approval 2014, updated March 2021).

“Pembrolizumab variant” as used herein means a monoclonal antibody that comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine or glycine residues of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“Pharmaceutical formulation” or “pharmaceutical composition” refers to preparations which are in such form as to permit the active ingredients to be effective, and which contains no additional components which are toxic to the subjects to which the formulation would be administered.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Pharmacokinetic “steady state” is a period of time during which any accumulation of drug concentrations owing to multiple doses has been maximized and systemic drug exposure is considered uniform after each subsequent dose administered; in the specific case of pembrolizumab, steady state is achieved at and after ~16 weeks of administration.

“Platinum-containing chemotherapy” (also known as platins) refers to the use of chemotherapeutic agent(s) used to treat cancer that are coordination complexes of platinum. Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

“RECIST 1.1 Response Criteria” as used herein means the definitions set forth in Eisenhauer, E. A. et al., Eur. J Cancer 45:228-247 (2009) for target lesions or non-target lesions, as appropriate based on the context in which response is being measured.

“Therapeutic agent” refers to an additional agent relative to the anti-PD-1 antibody or antigen-binding fragment thereof. A therapeutic agent may be, e.g., a chemotherapeutic, a biotherapeutic agent, or an immunogenic agent.

“Tissue section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor Mutational Burden” or “TMB” as used herein refers to the number of somatic mutations in a tumor's genome and/or the number of somatic mutations per area of the tumor's genome. TMB high (or TMB-H) refers to a tumor with a high mutational burden. In specific embodiments, a tumor is said to be TMB-H if it contains ≥10 mutations/megabase (Mut/Mb). An FDA approved test, such as FoundationOne® CDx is available for solid tumors to determine whether the solid tumor is TMB-H (i.e., has ≥10 mutations/megabase).

“Variable regions” or “V region” as used herein means the segment of IgG chains which is variable in sequence between different antibodies. It extends to Kabat residue 109 in the light chain and 113 in the heavy chain.

“PH 20” refers to the wild-type PH20 hyaluronidase of SEQ ID NO: 16.

“PH20 variant” as used herein is a variant of PH20 that has amino acid residue substitutions including M345T, S347T, M348K, K349E, L352Q, L353A, L354I, D355K, N356E, E359D, and I361T in SEQ ID NO: 16.

A “PH20 variant fragment” or “PH20 variant fragment thereof” “or “fragment of a PH20 variant” is a PH20 variant that has either an N-terminus deletion of amino acid residues 1-36, 1-37, 1-38, 1-39, 1-40, 1-41, or 1-42 of SEQ ID NO: 16; and/or a C-terminus deletion of amino acid residues 455-509, 456-509, 457-509, 458-509, 459-509, 460-509, 461-509, 462-509, 463-509, 464-509, 465-509, 466-509, 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500-509, 501-509, 502-509, 503-509, 504-509, 505-509, 506-509, 507-509, 508-509, or 509, wherein the numbering is by reference to SEQ ID NO: 16.

“Unit” or “U” refers to One unit of Hyaluronidase activity: amount of PH20 variant or fragment thereof that causes a change in the optical density at 600 nm at conditions suitable for reaction of hyaluronic acid and the enzyme and calculated according to a calibration curve using an activity standard. An example of the assay is described in Example 4 of US 2022/0089738. Hyaluronic acid (HA) binds to albumin and the albumin-HA complex develops turbidity. When HA is hydrolyzed by hyaluronidase, turbidity of albumin-HA complex is reduced. As such, this assay measures turbidity to determine hyaluronidase enzyme activity of PH20 variants or fragments thereof. Hyaluronidase activity is based on the following reaction: Hyaluronic acid->Di- and monosaccharides+smaller hyaluronic acid fragments. One skilled in the art understands that the hyaluronidase activity in Units per mg of hyaluronidase can vary depending on the purity, manufacturing process etc. of the hyaluronidase.

### II. PD-1 Antibodies and Antigen Binding Fragments Useful in the Invention

Examples of mAbs that bind to human PD-1, useful in the formulations, treatment methods, compositions, and uses of the invention, are described in U.S. Pat. Nos. 7,521,051, 8,008,449, and 8,354,509. Specific anti-human PD-1 mAbs useful as the PD-1 antagonist or the anti-PD-1 antibody in the treatment methods, compositions, and uses of the invention include: pembrolizumab (formerly known as MK-3475, SCH 900475 and lambrolizumab), a humanized IgG4 mAb with the structure described in WHO Drug Information, Vol. 27, No. 2, pages 161-162 (2013).

In specific embodiments of the treatment methods, compositions, kits and uses of the invention, the anti-PD-1 antibody, or antigen binding fragment thereof, comprises: (a) light chain CDRs LC-CDR1, LC-CDR2 and LC-CDR3 comprising a sequence of amino acids as set forth in SEQ ID NOs: 1, 2 and 3, respectively, and heavy chain CDRs HC-CDR1, HC-CDR2 and HC-CDR3 comprising a sequence of amino acids as set forth in SEQ ID NOs: 6, 7 and 8, respectively. In other embodiments, the anti-PD-1 antibody, or antigen binding fragment thereof, is a humanized antibody. In other embodiments, the anti-PD-1 antibody, or antigen binding fragment thereof, is a chimeric antibody. In specific embodiments, the anti-PD-1 antibody, or antigen binding fragment thereof, is a monoclonal antibody.

In other embodiments of the treatment methods, compositions, kits and uses of the invention, the anti-PD-1 antibody, or antigen binding fragment thereof, specifically binds to human PD-1 and comprises (a) a heavy chain variable region comprising an amino acid sequence as set forth in SEQ ID NO: 9, or a variant thereof, and (b) a light chain variable region comprising an amino acid sequence selected from the group consisting of SEQ ID NO: 4 or a variant thereof. In one embodiment, the anti-PD-1 antibody, or antigen binding fragment thereof that specifically binds to human PD-1 comprises (a) a heavy chain variable region comprising an amino acid sequence as set forth in SEQ ID NO: 9, and (b) a light chain variable region comprising an amino acid sequence of SEQ ID NO: 4.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitutions in the framework region.

In another embodiment of the treatment methods, compositions, kits and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in any one of SEQ ID NO: 10-15, or a variant thereof; and (b) a light chain comprising or consisting of a sequence of amino acids as set forth in SEQ ID NO: 5, or a variant thereof. In one embodiment, the anti-PD-1 antibody or antigen-binding fragment thereof is a monoclonal antibody which specifically binds to human PD-1 and comprises (a) a heavy chain consisting of a sequence of amino acids as set forth in any one of SEQ ID NO: 10-15; and (b) a light chain consisting of a sequence of amino acids as set forth in SEQ ID NO: 5.

In yet another embodiment of the treatment methods, compositions, kits and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO: 11 and (b) a light chain comprising or consisting of a sequence of amino acids as set forth in SEQ ID NO: 5.

Table 3 below provides a list of the amino acid sequences of exemplary anti-PD-1 mAbs for use in the treatment methods, compositions, kits and uses of the invention.

TABLE 3
Exemplary anti-PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |
| Heavy | XVQLVQSGVEVKKPGASVKVSCKASGYTFTNYYMYWV | 11 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLG | |
| | X is pyroglutamate | |
| Heavy | XVQLVQSGVEVKKPGASVKVSCKASGYTFTNYYMYWV | 12 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |
| | X is pyroglutamate | |
| Heavy | QVQLVQSGVEVKKPGASVKVSCKASGYTFTNYYMYWV | 13 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLG | |
| Heavy | XVQLVQSGVEVKKPGASVKVSCKASGYTFTNYYMYWV | 14 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSL | |
| | X is pyroglutamate | |
| Heavy | QVQLVQSGVEVKKPGASVKVSCKASGYTFTNYYMYWV | 15 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSL | |

As known to those skilled in the art, pyroglutamic acid is the conjugate acid of pyroglutamate, and is in equilibrium with pyroglutamate in solution.

### III. Human Hyaluronidase

It is known that there are six types of hyaluronidases in humans: Hyal1, Hyal2, Hyal3, Hyal4, HyalPS1, and PH20/SPAM1. Recombinant forms of these hyaluronidases with modifications, mutations, addition, truncations can be used in the disclosed methods, uses, compositions, and kits. See, e.g., U.S. Pat. Nos. 7,767,429, 8,431,380, 7,871,607, International Publication No. WO 2020/022791, U.S. Patent Publication No. US2006/0104968 and European Patent 1858926, and in numerous other patents and publications. Exemplary of such agents is the known agent PEGPH20 or rHuPH20. The methods, uses, compositions and kits of the invention encompass the use of any human hyaluronidase or fragments thereof, or variants or fragments thereof.

### PH20 Variants and Fragments Thereof

In one embodiment, the PH20 variant or fragment thereof further comprises an amino acid residue substitution at one or more positions selected from the group consisting of T341, L342, S343, 1344, and N363. In one embodiment, the PH20 variant or fragment thereof further comprises one or more amino acid residue substitutions selected from the group consisting of T341A, T341C, T341D, T341G, T341S, L342W, S343E, I344N and N363G.

In one embodiment of the PH20 variant or fragment thereof, the amino acid residue substitutions are selected from the group consisting of the following amino acid residue substitution groups:
(a) T341S, L342W, S343E, I344N, M345T, S347T, M348K, K349E, L352Q, L353A, L354I, D355K, N356E, E359D and I361T;
(b) L342W, S343E, I344N, M345T, S347T, M348K, K349E, L352Q, L353A, L354I, D355K, N356E, E359D and I361T;
(c) M345T, S347T, M348K, K349E, L352Q, L353A, L354I, D355K, N356E, E359D, I361T and N363 G;
(d) T341G, L342W, S343E, I344N, M345T, S347T, M348K, K349E, L352Q, L353A, L354I, D355K, N356E, E359D and I361T;
(e) T341A, L342W, S343E, I344N, M345T, S347T, M348K, K349E, L352Q, L353A, L354I, D355K, N356E, E359D and I361T;
(f) T341C, L342W, S343E, I344N, M345T, S347T, M348K, K349E, L352Q, L353A, L354I, D355K, N356E, E359D and I361T;
(g) T341D, L342W, S343E, I344N, M345T, S347T, M348K, K349E, L352Q, L353A, L354I, D355K, N356E, E359D and I361T;
(h) I344N, M345T, S347T, M348K, K349E, L352Q, L353A, L354I, D355K, N356E, E359D and I361T; and
(i) S343E, I344N, M345T, S347T, M348K, K349E, L352Q, L353A, L354I, D355K, N356E, E359D and I361T.

In one embodiment of the PH20 variant or fragment thereof, the amino acid residue substitutions consists of: T341S, L342W, S343E, I344N, M345T, S347T, M348K, K349E, L352Q, L353A, L354I, D355K, N356E, E359D and I361T.

In one aspect of the foregoing embodiments of a PH20 variant fragment, the PH20 variant fragment has an N-terminus deletion of amino acid residues 1-36, 1-37, 1-38, 1-39, 1-40, 1-41, or 1-42 of SEQ ID NO: 16. In another embodiment, the PH20 variant fragment has an N-terminus deletion of amino acid residues 1-36 of SEQ ID NO: 16. In another embodiment, the PH20 variant fragment has an N-terminus deletion of amino acid residues 1-37 of SEQ ID NO: 16. In another embodiment, the PH20 variant fragment has an N-terminus deletion of amino acid residues 1-38 of SEQ ID NO: 16.

In another aspect of the foregoing embodiments of a PH20 variant fragment, the PH20 variant fragment has a C-terminus deletion of amino acid residue(s) 455-509, 456-509, 457-509, 458-509, 459-509, 460-509, 461-509, 462-509, 463-509, 464-509, 465-509, 466-509, 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500-509, 501-509, 502-509, 503-509, 504-509, 505-509, 506-509, 507-509, 508-509, or 509, wherein the numbering is in reference to SEQ ID NO: 16. In one embodiment, the PH20 variant fragment thereof has a C-terminus deletion of amino acid residues 455-509, 458-509, 461-509, 464-509, 465-509, 466-509, 467-509, 468-509, 470-509, 471-509, 472-509, 473-509, 474-509, 475-509, 476-509, 478-509, 480-509, 482-509, 484-509, 486-509, 488-509, or 490-509, wherein the numbering is in reference to SEQ ID NO: 16. In one embodiment, the PH20 variant fragment has a C-terminus deletion of amino acid residues 468-509, wherein the numbering is in reference to SEQ ID NO: 16.

In one embodiment, the PH20 variant fragment consists of the amino acid sequence set forth in SEQ ID NO: 17 or 18. In other embodiments, the PH20 variant or fragment thereof is any of the sequences disclosed in Table 11 of EP3636752.

TABLE 4
Hyaluronidase and exemplary variants

| | | SEQ |
|---|---|---|
| | | ID |
| Protein | Sequence | NO: |
| Wild- | MGVLKFKHIFFRSFVKSSGVSQIVFTFLLIPCCLTLNFRAPPVIPNVPFL | 16 |
| type | WAWNAPSEFCLGKFDEPLDMSLFSFIGSPRINATGQGVTIFYVDRLGY | |
| PH20 | YPYIDSITGVTVNGGIPQKISLQDHLDKAKKDITFYMPVDNLGMAVID | |
| | WEEWRPTWARNWKPKDVYKNRSIELVQQQNVQLSLTEATEKAKQEF | |
| | EKAGKDFLVETIKLGKLLRPNHLWGYYLFPDCYNHHYKKPGYNGSCFN | |
| | VEIKRNDDLSWLWNESTALYPSIYLNTQQSPVAATLYVRNRVREAIRVS | |
| | KIPDAKSPLPVFAYTRIVFTDQVLKFLSQDELVYTFGETVALGASGIVIWG | |
| | TLSIMRSMKSCLLLDNYMETILNPYIINVTLAAKMCSQVLCQEQGVCIRKN | |
| | WNSSDYLHLNPDNFAIQLEKGGKFTVRGKPTLEDLEQFSEKFYCSCYSTLS | |
| | CKEKADVKDTDAVDVCIADGVCIDAFLKPPMETEEPQI | |
| | FYNASPSTLSATMFIVSILFLIISSVASL | |
| PH20 | LNFRAPPVIPNVPFLWAWNAPSEFCLGKFDEPLDMSLFSFIGSPRINATG | 17 |
| variant | QGVTIFYVDRLGYYPYIDSITGVTVNGGIPQKISLQDHLDKAKKDITFYM | |
| 1 | PVDNLGMAVIDWEEWRPTWARNWKPKDVYKNRSIELVQQQNVQLSLT | |
| | EATEKAKQEFEKAGKDFLVETIKLGKLLRPNHLWGYYLFPDCYNHHYKK | |
| | PGYNGSCFNVEIKRNDDLSWLWNESTALYPSIYLNTQQSPVAATLYVRNR | |
| | VREAIRVSKIPDAKSPLPVFAYTRIVFTDQVLKFLSQDELVYTFGETVALGA | |
| | SGIVIWGTLSITRTKESCQAIKEYMDTTLNPYIINVTLAAKMCSQVLCQEQG | |
| | VCIRKNWNSSDYLHLNPDNFAIQLEKGGKFTVRGKPTLEDLEQFSEKFYC | |
| | SCYSTLSCKEKADVKDTDAVDVCIADGVCIDAFLKPPMETEEPQIFYNASP | |
| | STLS | |
| PH20 | FRAPPVIPNVPFLWAWNAPSEFCLGKFDEPLDMSLFSFIGSPRINATGQGVTI | 18 |
| variant | FYVDRLGYYPYIDSITGVTVNGGIPQKISLQDHLDKAKKDITFYMPVDNLG | |
| 2 | MAVIDWEEWRPTWARNWKPKDVYKNRSIELVQQQNVQLSLTEATEKAKQ | |
| | EFEKAGKDFLVETIKLGKLLRPNHLWGYYLFPDCYNHHYKKPGYNGSCFN | |
| | VEIKRNDDLSWLWNESTALYPSIYLNTQQSPVAATLYVRNRVREAIRVSKIP | |
| | DAKSPLPVFAYTRIVFTDQVLKFLSQDELVYTFGETVALGASGIVIWGSWEN | |
| | TRTKESCQAIKEYMDTTLNPYIINVTLAAKMCSQVLCQEQGVCIRKNWNSS | |
| | DYLHLNPDNFAIQLEKGGKFTVRGKPTLEDLEQFSEKFYCSCYSTLSCKEKA | |
| | DVKDTDAVDVCIADGVCIDAF | |

In one aspect of the methods, compositions, kits and uses of the invention, the pharmaceutical composition comprises about 165 mg/mL of the anti-human PD-1 antibodies, or antigen binding fragments thereof, about 10 mM histidine buffer; about 10 mM L-methionine, or a pharmaceutically acceptable salt thereof, about 7% w/v sucrose, about 0.02% w/v polysorbate 80, and a PH20 variant or fragment. In one embodiment, the pharmaceutical composition comprises about 130 mg/mL of the anti-human PD-1 antibodies, or antigen binding fragments, thereof, about 10 mM histidine buffer, about 10 mM L-methionine, or a pharmaceutically acceptable salt thereof, about 7% w/v sucrose, about 0.02% w/v polysorbate 80, and a PH20 variant or fragment.

In specific embodiments of the pharmaceutical composition, the PH20 variant or fragment thereof is present in a concentration of about 1000 U/ml. In another embodiment, the concentration of the PH20 variant or fragment thereof is about 1500 U/ml. In another embodiment, the concentration of the PH20 variant or fragment thereof is about 2000 U/ml. In another embodiment, the concentration of the PH20 variant or fragment thereof is about 3000 U/ml. In another embodiment, the concentration of the PH20 variant or fragment thereof is about 4000 U/ml. In another embodiment, the concentration of the PH20 variant or fragment thereof is about 5000 U/ml. In another embodiment, the concentration of the PH20 variant or fragment thereof is about 6000 U/ml. In a further embodiment, the concentration of the PH20 variant or fragment thereof is about 1000-6000 U/ml. In a further embodiment, the concentration of the PH20 variant or fragment thereof is about 2000-5000 U/ml.

In specific embodiments of the pharmaceutical composition, the PH20 variant or fragment thereof is present in a concentration of about 150 U/ml. In another embodiment, the concentration of the PH20 variant or fragment thereof is about 300 U/ml. In another embodiment, the concentration of the PH20 variant or fragment thereof is about 600 U/ml. In another embodiment, the concentration of the PH20 variant or fragment thereof is about 750 U/ml. In a further embodiment, the concentration of the PH20 variant or fragment thereof is about 150-5000 U/ml. In a further embodiment, the concentration of the PH20 variant or fragment thereof is about 500 to 8000 U/ml.

In other aspects of the methods, compositions, kits and uses of the invention, the pharmaceutical composition comprises about 165 mg/mL of the anti-human PD-1 antibodies, and 2000 U/ml PH20 variant or fragment. In one embodiment, the pharmaceutical composition comprises about 130 mg/mL of the anti-human PD-1 antibodies, and 2000 U/ml PH20 variant or fragment.

rHuPH20 and Fragments Thereof

rHuPH20, also known as Hylenex®, consists of the amino acid sequence in SEQ ID NO: 20, which is amino acid residues 36-482 of wild-type human PH20 in SEQ ID NO:16 (amino acid residues 1-36 is the signal peptide sequence). In one embodiment, the rHuPH20 or variant or fragment is amino acid residues 36-464, 36-465, 36-466, 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO: 16; amino acid residues 37-464, 37-465, 37-466, 37-467, 37-468, 37-469, 37-470, 37-471, 37-472, 37-473, 37-474, 37-475, 37-476, 37-477, 37-478, 37-479, 37-480, 37-481, 37-482, or 37-483 of SEQ ID NO: 16; amino acid residues 38-464, 38-465, 38-466, 38-467, 38-468, 38-469, 38-470, 38-471, 38-472, 38-473, 38-474, 38-475, 38-476, 38-477, 38-478, 38-479, 38-480, 38-481, 38-482, or 38-483 of SEQ ID NO: 16; amino acid residues 39-464, 39-465, 39-466, 39-467, 39-468, 39-469, 39-470, 39-471, 39-472, 39-473, 39-474, 39-475, 39-476, 39-477, 39-478, 39-479, 39-480, 39-481, 39-482, or 39-483 of SEQ ID NO: 16; amino acid residues 40-464, 40-465, 40-466, 40-467, 40-468, 40-469, 40-470, 40-471, 40-472, 40-473, 40-474, 40-475, 40-476, 40-477, 40-478, 40-479, 40-480, 40-481, 340-482, or 40-483 of SEQ ID NO: 16; amino acid residues 41-464, 41-465, 41-466, 41-467, 41-468, 41-469, 41-470, 41-471, 41-472, 41-473, 41-474, 41-475, 41-476, 41-477, 41-478, 41-479, 41-480, 41-481, 41-482, or 41-483 of SEQ ID NO: 16; or amino acid residues 42-464, 42-465, 42-466, 42-467, 42-468, 42-469, 42-470, 42-471, 42-472, 42-473, 42-474, 42-475, 42-476, 42-477, 42-478, 42-479, 42-480, 42-481, 42-482, or 42-483 of SEQ ID NO: 16. In a preferred embodiment, the rHuPH20 variant consists of amino acid residues 36-483 of SEQ ID NO: 16 (which is SEQ ID NO: 19). In a preferred embodiment, the rHuPH20 fragment consists of amino acid residues 36-477 of SEQ ID NO: 16. In a preferred embodiment, the rHuPH20 fragment consists of amino acid residues 36-478 of SEQ ID NO: 16. In a preferred embodiment, the rHuPH20 fragment consists of amino acid residues 36-479 of SEQ ID NO: 16. In a preferred embodiment, the rHuPH20 fragment consists of amino acid residues 36-480 of SEQ ID NO: 16. In a preferred embodiment, the rHuPH20 fragment consists of amino acid residues 36-481 of SEQ ID NO: 16. In further embodiments, the rHuPH20 variant or fragment are those disclosed in U.S. Pat. No. 7,767,429, incorporated herein by reference in its entirety.

TABLE 5
Hyaluronidase and exemplary variants

| | | SEQ |
|---|---|---|
| | | ID |
| Protein | Sequence | NO: |
| rHuPH20 | LNFRAPPVIPNVPFLWAWNAPSEFCLGKFDEPLDMSLFSFIGSPRIN | 19 |
| variant 1 | ATGQGVTIFYVDRLGYYPYIDSITGVTVNGGIPQKISLQDHLDKAK | |
| | KDITFYMPVDNLGMAVIDWEEWRPTWARNWKPKDVYKNRSIEL | |
| | VQQQNVQLSLTEATEKAKQEFEKAGKDFLVETIKLGKLLRPNHL | |
| | WGYYLFPDCYNHHYKKPGYNGSCFNVEIKRNDDLSWLWNESTA | |
| | LYPSIYLNTQQSPVAATLYVRNRVREAIRVSKIPDAKSPLPVFAYT | |
| | RIVFTDQVLKFLSQDELVYTFGETVALGASGIVIWGTLSIMRSMKS | |
| | CLLLDNYMETILNPYIINVTLAAKMCSQVLCQEQGVCIRKNWNSS | |
| | DYLHLNPDNFAIQLEKGGKFTVRGKPTLEDLEQFSEKFYCSCYSTL | |
| | SCKEKADVKDTDAVDVCIADGVCIDAFLKPPMETEEPQIFYN | |
| rHuPH20 | LNFRAPPVIPNVPFLWAWNAPSEFCLGKFDEPLDMSLFSFIGSPRIN | 20 |
| | ATGQGVTIFYVDRLGYYPYIDSITGVTVNGGIPQKISLQDHLDKAK | |
| | KDITFYMPVDNLGMAVIDWEEWRPTWARNWKPKDVYKNRSIEL | |
| | VQQQNVQLSLTEATEKAKQEFEKAGKDFLVETIKLGKLLRPNHL | |
| | WGYYLFPDCYNHHYKKPGYNGSCFNVEIKRNDDLSWLWNESTA | |
| | LYPSIYLNTQQSPVAATLYVRNRVREAIRVSKIPDAKSPLPVFAYT | |
| | RIVFTDQVLKFLSQDELVYTFGETVALGASGIVIWGTLSIMRSMKS | |
| | CLLLDNYMETILNPYIINVTLAAKMCSQVLCQEQGVCIRKNWNSS | |
| | DYLHLNPDNFAIQLEKGGKFTVRGKPTLEDLEQFSEKFYCSCYSTL | |
| | SCKEKADVKDTDAVDVCIADGVCIDAFLKPPMETEEPQIFY | |

In one aspect, the methods, compositions, kits and uses of the invention use a pharmaceutical composition that comprises about 165 mg/mL of the anti-human PD-1 antibodies, about 10 mM histidine buffer; about 10 mM L-methionine, or a pharmaceutically acceptable salt thereof, about 7% w/v sucrose, about 0.02% w/v polysorbate 80, and a rHuPH20 or variant or fragment. In one embodiment, the pharmaceutical composition comprises about 130 mg/mL of the anti-human PD-1 antibodies, or antigen binding fragments thereof, about 10 mM histidine buffer, about 10 mM L-methionine, or a pharmaceutically acceptable salt thereof, about 7% w/v sucrose, about 0.02% w/v polysorbate 80, and a rHuPH20 or variant or fragment.

In specific embodiments of the pharmaceutical compositions, the rHuPH20 or variant or fragment thereof is present in a concentration of about 1000 U/ml. In another embodiment, the concentration of the rHuPH20 or variant or fragment thereof is about 1500 U/ml. In another embodiment, the concentration of the rHuPH20 or variant or fragment thereof is about 2000 U/ml. In another embodiment, the concentration of the rHuPH20 or variant or fragment thereof is about 3000 U/ml. In another embodiment, the concentration of the rHuPH20 or variant or fragment thereof is about 4000 U/ml. In another embodiment, the concentration of the rHuPH20 or variant or fragment thereof is about 5000 U/ml. In another embodiment, the concentration of the rHuPH20 or variant or fragment thereof is about 6000 U/ml. In a further embodiment, the concentration of the rHuPH20 or variant or fragment thereof is about 1000-6000 U/ml. In a further embodiment, the concentration of the rHuPH20 or variant or fragment thereof is about 2000-5000 U/ml.

In specific embodiments of the pharmaceutical composition, the rHuPH20 or variant or fragment thereof is present in a concentration of about 150 U/ml. In another embodiment, the concentration of the rHuPH20 or variant or fragment thereof is about 300 U/ml. In another embodiment, the concentration of the rHuPH20 or variant or fragment thereof is about 600 U/ml. In another embodiment, the concentration of the rHuPH20 or variant or fragment thereof is about 750 U/ml. In a further embodiment, the concentration of the rHuPH20 or variant or fragment thereof is about 150-5000 U/ml. In a further embodiment, the concentration of the rHuPH20 or variant or fragment thereof is about 500 to 8000 U/ml.

In another aspect, the methods, compositions, kits and uses of the invention use a pharmaceutical composition that comprises about 165 mg/mL of the anti-human PD-1 antibodies, or antigen binding fragments thereof, and about 2000 U/ml of a rHuPH20 or variant or fragment. In one embodiment, the pharmaceutical composition comprises about 130 mg/mL of the anti-human PD-1 antibodies, or antigen binding fragments thereof, and about 2000 U/ml of a rHuPH20 or variant or fragment.

### IV. Methods and Uses of the Invention

The invention provides a method of treating cancer in a human patient in need thereof comprising subcutaneously administering to the patient a dose of from about 600 mg to about 1000 mg of an anti-PD-1 antibody, or antigen binding fragment thereof, and a human hyaluronidase, every approximately six weeks. In another aspect, the invention provides use of an anti-PD-1 antibody, or antigen binding fragment thereof and a human hyaluronidase for the preparation of a medicament for the treatment of cancer in a human patient, wherein the patient is subcutaneously administered a dose of from about 600 mg to about 1000 mg of an anti-PD-1 antibody, or antigen binding fragment thereof every approximately six weeks. In another aspect, the invention provides use of an anti-PD-1 antibody, or antigen binding fragment thereof for the preparation of a medicament for the treatment of cancer in a human patient, wherein the patient is co-administered subcutaneously a dose of from about 600 mg to about 1000 mg of an anti-PD-1 antibody, or antigen binding fragment thereof and a human hyaluronidase every approximately six weeks. In another aspect, the invention provides a pharmaceutical composition comprising an anti-PD-1 antibody, or antigen binding fragment thereof and a human hyaluronidase for the treatment of cancer in a human patient, wherein the patient is subcutaneously administered a dose of from about 600 mg to about 1000 mg of the anti-PD-1 antibody, or antigen binding fragment thereof and a human hyaluronidase every approximately six weeks. In another aspect, the invention provides a pharmaceutical composition comprising an anti-PD-1 antibody, or antigen binding fragment thereof for the treatment of cancer in a human patient, wherein the patient is co-administered subcutaneously a dose of from about 600 mg to about 1000 mg of the anti-PD-1 antibody, or antigen binding fragment thereof and a human hyaluronidase every approximately six weeks.

The invention also provides a method of treating cancer in a human patient in need thereof comprising subcutaneously administering to the patient a dose of from about 600 mg to about 1000 mg of an anti-PD-1 antibody, or antigen binding fragment thereof every approximately six weeks. In another aspect, the invention provides use of an anti-PD-1 antibody, or antigen binding fragment thereof for the preparation of a medicament for the treatment of cancer in a human patient, wherein the patient is subcutaneously administered a dose of from about 600 mg to about 1000 mg of an anti-PD-1 antibody, or antigen binding fragment thereof every approximately six weeks. In another aspect, the invention provides an anti-PD-1 antibody, or antigen binding fragment thereof for the treatment of cancer in a human patient, wherein the patient is subcutaneously administered a dose of from about 600 mg to about 1000 mg of the anti-PD-1 antibody, or antigen binding fragment thereof every approximately six weeks.

The invention further provides a method of treating cancer in a human patient in need thereof comprising subcutaneously administering to the patient about 300 mg to about 500 mg of an anti-PD-1 antibody, or antigen binding fragment thereof and a human hyaluronidase, every approximately three weeks. In another aspect, the invention provides use of an anti-PD-1 antibody, or antigen binding fragment thereof and a human hyaluronidase for the preparation of a medicament for the treatment of cancer in a human patient, wherein the patient is subcutaneously administered a dose of from about 300 mg to about 500 mg of an anti-PD-1 antibody, or antigen binding fragment thereof and a human hyaluronidase every approximately three weeks. In another aspect, the invention provides use of an anti-PD-1 antibody, or antigen binding fragment thereof for the preparation of a medicament for the treatment of cancer in a human patient, wherein the patient is co-administered subcutaneously a dose of from about 300 mg to about 500 mg of an anti-PD-1 antibody, or antigen binding fragment thereof and a human hyaluronidase every approximately three weeks. In another aspect, the invention provides a pharmaceutical composition comprising an anti-PD-1 antibody, or antigen binding fragment thereof and a human hyaluronidase for the treatment of cancer in a human patient, wherein the patient is subcutaneously administered a dose of from about 300 mg to about 500 mg of the anti-PD-1 antibody, or antigen binding fragment thereof and a human hyaluronidase every approximately three weeks. In another aspect, the invention provides a pharmaceutical composition comprising an anti-PD-1 antibody, or antigen binding fragment thereof for the treatment of cancer in a human patient, wherein the patient is co-administered subcutaneously a dose of from about 300 mg to about 500 mg of the anti-PD-1 antibody, or antigen binding fragment thereof and a human hyaluronidase every approximately three weeks.

In particular embodiments of the invention, the anti-PD-1 antibody, or antigen-binding fragment thereof, is pembrolizumab. In other embodiments, the anti-PD-1 antibody, or antigen-binding fragment thereof, is a pembrolizumab variant.

In embodiments of any of the methods or uses of the invention, the bioavailability of the anti-PD-1 antibody, or antigen binding fragment thereof in combination with the human hyaluronidase is 55-60%. In embodiments of any of the methods or uses described herein, the bioavailability of the anti-PD-1 antibody, or antigen binding fragment thereof in combination with the human hyaluronidase, is 57-59%. In embodiments of any of the methods or uses described herein, the bioavailability of the anti-PD-1 antibody, or antigen binding fragment thereof in combination with the human hyaluronidase is 57%.

In embodiments of any of the methods or uses of the invention, the subcutaneous administration of the anti-PD-1 antibody, or antigen binding fragment thereof results in a C_{trough} that is within 20% of the C_{trough} of a 400 mg dose of the anti-PD-1 antibody, or antigen binding fragment thereof, administered by an intravenous (IV) route of administration every 6 weeks. In another embodiment, the subcutaneous administration of the anti-PD-1 antibody, or antigen binding fragment thereof results in a C_{trough} that is at least the same as, or less than 35% greater than, the C_{trough} of a 400 mg dose of the anti-PD-1 antibody, or antigen binding fragment thereof, administered by an intravenous (IV) route of administration every 6 weeks. In another embodiment, the subcutaneous administration of the anti-PD-1 antibody, or antigen binding fragment thereof results in a C_{trough} that is at least the same as, or less than 30% greater than, the C_{trough} of a 400 mg dose of the anti-PD-1 antibody, or antigen binding fragment thereof, administered by an intravenous (IV) route of administration every 6 weeks. In another embodiment, the subcutaneous administration of the anti-PD-1 antibody, or antigen binding fragment thereof results in a C_{trough} that is about 25-30% greater than, the C_{trough} of a 400 mg dose of the anti-PD-1 antibody, or antigen binding fragment thereof, administered by an intravenous (IV) route of administration every 6 weeks. In another embodiment, the subcutaneous administration of the anti-PD-1 antibody, or antigen binding fragment thereof results in a C_{trough} that is about 30% greater than, the C_{trough} of a 400 mg dose of the anti-PD-1 antibody, or antigen binding fragment thereof, administered by an intravenous (IV) route of administration every 6 weeks. In another embodiment, the subcutaneous administration of the anti-PD-1 antibody, or antigen binding fragment thereof results in a C_{trough} that is about 20-35% greater than, the C_{trough} of a 400 mg dose of the anti-PD-1 antibody, or antigen binding fragment thereof, administered by an intravenous (IV) route of administration every 6 weeks.

In specific embodiments of the methods or uses of the invention, the subcutaneous administration of the dose of the anti-PD-1 antibody (e.g., pembrolizumab), or antigen binding fragment thereof, results in a C_{trough} that is the same, or greater than, the C_{trough} of the dose administered by 400 mg Q6W IV route of administration. In an embodiment of any of the methods or uses described herein, the subcutaneous administration of the anti-PD-1 antibody, or antigen binding fragment thereof, results in a ratio of subcutaneous C_{trough} to IV C_{trough} (e.g. geometric mean ratio) of at least 0.8, at least 1, at least 1.2, at least 1.3, at least 1.4, at least 1.5, or at least 1.6. In specific embodiments, the subcutaneous administration results in a PK profile having a SC:IV C_{trough} ratio of at least 0.8 or greater. In specific embodiments, the subcutaneous administration results in a PK profile having a SC:IV C_{trough} ratio of at least 1.0 or greater. In specific embodiments, the subcutaneous administration results in a SC:IV C_{trough} ratio of at least 1.2 or greater. In specific embodiments, the subcutaneous administration results in a SC:IV C_{trough} ratio of at least 1.3 or greater. In specific embodiments, the subcutaneous administration results in a SC:IV C_{trough} ratio of at least 1.4 or greater. In specific embodiments, the subcutaneous administration results in a SC:IV C_{trough} ratio of at least 1.5 or greater. In specific embodiments, the subcutaneous administration results in a SC:IV C_{trough} ratio of at least 1.6 or greater.

In specific embodiments of the methods or uses of the invention, the subcutaneous administration of the dose of the anti-PD-1 antibody (e.g., pembrolizumab), or antigen binding fragment thereof, results in a SC:IV C_{trough} ratio of 0.8 to 1.6, 1.0 to 1.6, 1.1 to 1.6, 1.2 to 1.6, 1.3 to 1.6, 1.4 to 1.6, 1.2 to 1.5, 1.3 to 1.5, 1.4 to 1.5 or 1.3 to 1.4.

In specific embodiments of the methods or uses of the invention, the subcutaneous administration of the dose of anti-PD-1 antibody (e.g., pembrolizumab), or antigen binding fragment thereof, results in a SC:IV C_{trough} ratio of 1.0 to 1.6. In specific embodiments, the subcutaneous administration results in a SC:IV C_{trough} ratio of 1.1 to 1.6. In specific embodiments, the subcutaneous administration results in a SC:IV C_{trough} ratio of 1.2 to 1.6. In specific embodiments, the subcutaneous administration results in a SC:IV C_{trough} ratio of 1.3 to 1.6. In specific embodiments, the subcutaneous administration results in a SC:IV C_{trough} ratio of 1.4 to 1.6. In specific embodiments, the subcutaneous administration results in a SC:IV C_{trough} ratio of at 1.2 to 1.5. In specific embodiments, the subcutaneous administration results in a SC:IV C_{trough} ratio of at 1.3 to 1.5. In specific embodiments, the subcutaneous administration results in a SC:IV C_{trough} ratio of at 1.4 to 1.5. In specific embodiments, the subcutaneous administration results in a SC:IV C_{trough} ratio of at 1.3 to 1.4. In specific embodiments, the subcutaneous administration results in a SC:IV C_{trough} ratio of at about 1.2 to 1.3. In specific embodiments, the subcutaneous administration results in a SC:IV C_{trough} ratio of at about 1.2 to 1.4.

In specific embodiments of the methods or uses of the invention, the subcutaneous administration of the dose of anti-PD-1 antibody (e.g., pembrolizumab), or antigen binding fragment thereof, results in a AUC_{(0-6 weeks)} that is at least 0.8 ratio of the AUC_{(0-6 weeks)} of a 400 mg dose of the anti-PD-1 antibody, or antigen binding fragment thereof, administered by a Q6W IV route of administration at cycle 1 or steady state. In specific embodiments of the methods or uses of the invention, the subcutaneous administration of the dose of anti-PD-1 antibody (e.g., pembrolizumab), or antigen binding fragment thereof, results in an AUC_{(0-6 weeks)} of about a 1.0 ratio of the AUC_{(0-6 weeks)} of a 400 mg dose of the anti-PD-1 antibody, or antigen binding fragment thereof, administered by a Q6W IV route of administration during treatment (e.g. at cycle 1 or steady state). In specific embodiments of the methods or uses of the invention, the subcutaneous administration of the dose of anti-PD-1 antibody (e.g., pembrolizumab), or antigen binding fragment thereof, results in an AUC_{(0-6 weeks)} of about 0.8 to 1.2 ratio of the AUC_{(0-6 weeks)} of a 400 mg dose of the anti-PD-1 antibody, or antigen binding fragment thereof, administered by a Q6W IV route of administration during treatment (e.g., at cycle 1 or steady state). In specific embodiments of the methods or uses of the invention, the subcutaneous administration of the dose of anti-PD-1 antibody (e.g., pembrolizumab), or antigen binding fragment thereof, results in an AUC_{(0-6 weeks)} of about 1.0 to 1.1 ratio of the AUC_{(0-6 weeks)} of a 400 mg dose of the anti-PD-1 antibody, or antigen binding fragment thereof, administered by a Q6W IV route of administration during treatment (e.g. at cycle 1 or steady state). In specific embodiments, the subcutaneous administration results in a PK profile having a SC:IV AUC_{(0-6 weeks)} ratio of at least 0.8, 1.0 or greater after six cycles of administration.

In embodiments of any of the methods or uses of the invention, the subcutaneous administration of the anti-PD-1 antibody (e.g., pembrolizumab) co-formulated with a human hyaluronidase (e.g. PH20 variant 2) at 760-790 mg every six weeks results in a C_{trough} that is within 20% of the C_{trough} of a 400 mg dose of the anti-PD-1 antibody, administered by an intravenous (IV) route of administration every 6 weeks. In specific embodiments of the methods or uses of the invention, the subcutaneous administration of the anti-PD-1 antibody (e.g., pembrolizumab) co-formulated with a human hyaluronidase (e.g. PH20 variant 2) at 760-790 mg every six weeks, results in a ratio of about 1.0 to 1.35 subcutaneous C_{trough} to IV C_{trough} (e.g. geometric mean ratio) of the dose administered by 400 mg Q6W IV route of administration. In one embodiment of the foregoing embodiments, the subcutaneous administration of the anti-PD-1 antibody (e.g. pembrolizumab) is co-formulated with PH20 variant 2 and is at 790 mg every six weeks. In preferred embodiments of the methods or uses of the invention, the subcutaneous administration of the anti-PD-1 antibody (e.g., pembrolizumab) co-formulated with PH20 variant 2 at 790 mg every six weeks, results in a ratio of about 1.3 subcutaneous C_{trough} to IV C_{trough} (e.g. geometric mean ratio) of the dose administered by 400 mg Q6W IV route of administration.

In specific embodiments of the methods or uses of the invention, the subcutaneous administration of the anti-PD-1 antibody (e.g., pembrolizumab) co-formulated with a human hyaluronidase (e.g. PH20 variant 2) at 760-790 mg every six weeks results in an AUC_{(0-6 weeks)} of about 0.8 to 1.2 ratio of the AUC_{(0-6 weeks)} of a 400 mg dose of the anti-PD-1 antibody, administered by a Q6W IV route of administration during the treatment duration. In specific embodiments of the methods or uses of the invention, the subcutaneous administration of the dose of anti-PD-1 antibody (e.g., pembrolizumab) co-formulated with a human hyaluronidase (e.g. PH20 variant 2) at 760-790 mg every six weeks results in an AUC_{(0-6 weeks)} of about 1.0 to 1.2 ratio of the AUC_{(0-6 weeks)} of a 400 mg dose of the anti-PD-1 antibody, administered by a Q6W IV route of administration during the treatment duration. In specific embodiments of the methods or uses of the invention, the subcutaneous administration of anti-PD-1 antibody (e.g., pembrolizumab) co-formulated with a human hyaluronidase (e.g. PH20 variant 2) at 760-790 mg every six weeks results in an AUC_{(0-6 weeks)} of about 1.0 to 1.1 ratio of the AUC_{(0-6 weeks)} of a 400 mg dose of the anti-PD-1 antibody, administered by a Q6W IV route of administration during the treatment duration. In one embodiment of the foregoing embodiments, the subcutaneous administration of the anti-PD-1 antibody (e.g. pembrolizumab) is co-formulated with PH20 variant 2 and is at 790 mg every six weeks. In preferred embodiments of the methods or uses of the invention, the subcutaneous administration of the anti-PD-1 antibody (e.g., pembrolizumab) co-formulated with PH20 variant 2 at 790 mg every six weeks, results in an AUC_{(0-6 weeks)} of about 1.1 ratio of the AUC_{(0-6 weeks)} of the dose administered by 400 mg Q6W IV route of administration.

In embodiments of any of the methods or uses of the invention, the subcutaneous administration of the anti-PD-1 antibody (e.g. pembrolizumab) co-formulated with a human hyaluronidase (e.g. PH20 variant 2) at 760-790 mg every six weeks results in a Cₘₐₓ that is about 40% lower than the Cₘₐₓ of a 400 mg dose of the anti-PD-1 antibody, administered by an intravenous (IV) route of administration every 6 weeks at cycle 1. In embodiments of any of the methods or uses of the invention, the subcutaneous administration of the anti-PD-1 antibody (e.g. pembrolizumab) co-formulated with a human hyaluronidase (e.g. PH20 variant 2) at 760-790 mg every six weeks results in a Cₘₐₓ that is about 35-38% lower than the Cₘₐₓ of a 400 mg dose of the anti-PD-1 antibody, administered by an intravenous (IV) route of administration every 6 weeks at cycle 1. In embodiments of any of the methods or uses of the invention, the subcutaneous administration of the anti-PD-1 antibody (e.g pembrolizumab) co-formulated with a human hyaluronidase (e.g. PH20 variant 2) at 760-790 mg every six weeks results in a Cₘₐₓ that is about 20-30% lower than the Cₘₐₓ of a 400 mg dose of the anti-PD-1 antibody, or antigen binding fragment thereof, administered by an intravenous (IV) route of administration every 6 weeks at steady state. In embodiments of any of the methods or uses of the invention, the subcutaneous administration of the anti-PD-1 antibody (e.g. pembrolizumab) co-formulated with a human hyaluronidase (e.g. PH20 variant 2) at 760-790 mg every six weeks results in a Cₘₐₓ that is about 22-25% lower than the Cₘₐₓ of a 400 mg dose of the anti-PD-1 antibody, administered by an intravenous (IV) route of administration every 6 weeks at steady state. In one embodiment of the foregoing embodiments, the subcutaneous administration of the anti-PD-1 antibody (e.g. pembrolizumab) co-formulated with a human hyaluronidase (e.g. PH20 variant 2) is at 790 mg every six weeks. In embodiments of any of the methods or uses of the invention, the subcutaneous administration of the anti-PD-1 antibody (e.g. pembrolizumab) co-formulated with PH20 variant 2 at 790 mg every six weeks results in a Cₘₐₓ that is about 22% lower than the Cₘₐₓ of a 400 mg dose of the anti-PD-1 antibody, administered by an intravenous (IV) route of administration every 6 weeks at steady state. In embodiments of any of the methods or uses of the invention, the subcutaneous administration of the anti-PD-1 antibody (e.g. pembrolizumab) co-formulated with PH20 variant 2 at 790 mg every six weeks results in a Cₘₐₓ that is about 35% lower than the Cₘₐₓ of a 400 mg dose of the anti-PD-1 antibody, administered by an intravenous (IV) route of administration every 6 weeks at cycle 1.

In embodiments of any of the methods or uses of the invention, the subcutaneous administration of the anti-PD-1 antibody (e.g., pembrolizumab) co-formulated with a human hyaluronidase (e.g. PH20 variant 2) at 380-395 mg every three weeks results in a C_{trough} that is within 20% of the C_{trough} of a 400 mg dose of the anti-PD-1 antibody, administered by an intravenous (IV) route of administration every 6 weeks. In specific embodiments of the methods or uses of the invention, the subcutaneous administration of the anti-PD-1 antibody (e.g., pembrolizumab) co-formulated with a human hyaluronidase (e.g. PH20 variant 2) at 380-395 mg every three weeks, results in a ratio of about 1.0 to 2.0 subcutaneous C_{trough} to IV C_{trough} (e.g. geometric mean ratio) of the dose administered by 400 mg Q6W IV route of administration. In one embodiment of the foregoing embodiments, the subcutaneous administration of the anti-PD-1 antibody (e.g. pembrolizumab) co-formulated with PH20 variant 2 is at 395 mg every three weeks. In preferred embodiments of the methods or uses of the invention, the subcutaneous administration of the anti-PD-1 antibody (e.g., pembrolizumab) co-formulated with PH20 variant 2 at 395 mg every three weeks, results in a ratio of about 1.5-2.0 subcutaneous C_{trough} to IV C_{trough} (e.g. geometric mean ratio) of the dose administered by 400 mg Q6W IV route of administration.

In specific embodiments of the methods or uses of the invention, the subcutaneous administration of the anti-PD-1 antibody (e.g., pembrolizumab) co-formulated with a human hyaluronidase (e.g. PH20 variant 2) at 380-395 mg every three weeks results in an AUC_{(0-6 weeks)} of about 0.8 to 1.2 ratio of the AUC_{(0-6 weeks)} of a 400 mg dose of the anti-PD-1 antibody, administered by a Q6W IV route of administration during the treatment duration. In specific embodiments of the methods or uses of the invention, the subcutaneous administration of the dose of anti-PD-1 antibody (e.g., pembrolizumab) co-formulated with a human hyaluronidase (e.g. PH20 variant 2) at 380-395 mg every three weeks results in an AUC_{(0-6 weeks)} of about 0.8 to 1.1 ratio of the AUC_{(0-6 weeks)} of a 400 mg dose of the anti-PD-1 antibody, administered by a Q6W IV route of administration during the treatment duration. In one embodiment of the foregoing embodiments, the subcutaneous administration of the anti-PD-1 antibody (e.g. pembrolizumab) co-formulated with PH20 variant 2 is at 395 mg every three weeks. In preferred embodiments of the methods or uses of the invention, the subcutaneous administration of the anti-PD-1 antibody (e.g., pembrolizumab) co-formulated with PH20 variant 2 at 395 mg every three weeks, results in an AUC_{(0-6 weeks)} of about 1.1 ratio of the AUC_{(0-6 weeks)} of the dose administered by 400 mg Q6W IV route of administration.

In one embodiment of the foregoing embodiments, the ratio is a geometric mean ratio. In one embodiment of the foregoing embodiments, the ratio is at Cycle 1 (3 weeks or six weeks depending on the subcutaneous administration cycle). In one embodiment of the foregoing embodiments, the ratio is at steady state. In one embodiment of the foregoing embodiments, the ratio is during the treatment duration.

In specific embodiments of the methods or uses of the invention, the cancer is selected from the group consisting of: melanoma, lung cancer, head and neck cancer, bladder cancer, breast cancer, gastrointestinal cancer, multiple myeloma, hepatocellular cancer, merkel cell carcinoma, cutaneous squamous cell carcinoma, lymphoma, renal cancer, mesothelioma, ovarian cancer, esophageal cancer, anal cancer, biliary tract cancer, colorectal cancer, endometrial cancer, cervical cancer, thyroid cancer, salivary cancer, prostate cancer (e.g. hormone refractory prostate adenocarcinoma), pancreatic cancer, colon cancer, liver cancer, thyroid cancer, glioblastoma, glioma, and other neoplastic malignancies.

In specific embodiments the lung cancer is non-small cell lung cancer.

In specific embodiments, the lung cancer is small cell lung cancer.

In specific embodiments, the lymphoma is Hodgkin lymphoma.

In specific embodiments, the lymphoma is non-Hodgkin lymphoma. In specific embodiments, the lymphoma is primary mediastinal large B-cell lymphoma (PMBCL). In specific embodiments, the lymphoma is diffuse large B-cell lymphoma (DLBCL). In specific embodiments, the lymphoma is mantle cell lymphoma.

In specific embodiments, the breast cancer is triple negative breast cancer.

In specific embodiments, the breast cancer is ER+/HER2− breast cancer.

In specific embodiments, the breast cancer is HR+/HER2− breast cancer.

In specific embodiments, the breast cancer is HER2+ breast cancer.

In specific embodiments, the breast cancer is ER+ breast cancer.

In specific embodiments, the breast cancer is germline BRCA mutant HER2− breast cancer.

In specific embodiments, the bladder cancer is urothelial cancer.

In specific embodiments, the head and neck cancer is nasopharyngeal cancer. In specific embodiments, the cancer is thyroid cancer. In other embodiments, the cancer is salivary cancer. In other embodiments, the cancer is squamous cell carcinoma of the head and neck.

In specific embodiments, the cancer is metastatic colorectal cancer with high levels of microsatellite instability (MSI-H).

In specific embodiments, the cancer is microsatellite stable (MSS) colorectal cancer.

In specific embodiments, the cancer is a solid tumor with a high level of microsatellite instability (MSI-H).

In specific embodiments, the cancer is a solid tumor with a high mutational burden.

In specific embodiments of the methods or uses of the invention, the cancer is selected from the group consisting of: melanoma, non-small cell lung cancer, small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

In specific embodiments of the methods or uses of the invention, the cancer is selected from the group consisting of: melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

In specific embodiments of the methods or uses of the invention, the cancer is selected from the group consisting of melanoma, non-small cell lung cancer, head and neck squamous cell cancer, urothelial carcinoma, classical Hodgkin lymphoma, primary mediastinal large B-cell lymphoma (PMBCL), MSI-H cancer, MSI-H or Mismatch Repair Deficient colorectal cancer, gastric cancer, gastroesophageal junction adenocarcinoma, esophageal cancer, cervical cancer, hepatocellular cancer, merkel cell carcinoma, renal cell carcinoma, endometrial carcinoma, cutaneous squamous cell carcinoma, Tumor Mutational Burden-High (TMB-H) cancer, and triple negative breast cancer.

In a first embodiment (Embodiment E1), the invention comprises a method of treating cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every approximately six weeks. In specific embodiments, the anti-PD-1 antibody, or antigen binding fragment thereof, is administered once every six weeks.

In a second embodiment (Embodiment E2), the invention comprises a method of treating unresectable or metastatic melanoma in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In specific embodiments, the anti-PD-1 antibody, or antigen binding fragment thereof, is administered once every six weeks. In a third embodiment (Embodiment E3), the invention comprises a method of treating metastatic non-small cell lung cancer (NSCLC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In specific embodiments, the anti-PD-1 antibody, or antigen binding fragment thereof, is administered once every six weeks.

In a sub-embodiment of Embodiment E3 (Embodiment E3-A), the patient has a tumor with high PD-L1 expression [(Tumor Proportion Score (TPS)≥50%)] and was not previously treated with platinum-containing chemotherapy.

In a further sub-embodiment of Embodiment E3 (Embodiment E3-B), the patient has a tumor with PD-L1 expression (TPS ≥1%) and was previously treated with platinum-containing chemotherapy. In specific embodiments of Embodiment E3-B, the patient had disease progression on or after receiving platinum-containing chemotherapy, or at least one prior chemotherapy.

In another sub-embodiment of Embodiment E3 (Embodiment E3-C), the patient has a tumor with PD-L1 expression (TPS≥1%) and was not previously treated with platinum-containing chemotherapy. In one embodiment, the patient has Stage III non-small cell lung cancer, and are not candidates for surgical resection or definitive chemoradiation. In one embodiment, the patient has metastatic non-small cell lung cancer. In yet another sub-embodiment of Embodiment E3 (Embodiment E3-D), the patient's tumor is not tested for PD-L1 expression. In this embodiment, the patient is treated with the anti-PD-1 antibody, or antigen binding fragment thereof, regardless of PD-L1 expression. In specific embodiments, the patient was not previously treated with platinum-containing chemotherapy.

In certain embodiments of Embodiment E3 (including Embodiments E3-A, E3-B, and E3-C), the PD-L1 TPS is determined by an FDA-approved test.

In certain embodiments of Embodiment E3 (including Embodiments E3-A, E3-B, E3-C and E3-D), the patient's tumor has no EGFR or ALK genomic aberrations.

In certain embodiments of Embodiment E3 (including Embodiments E3-A, E3-B, E3-C and E3-D), the patient's tumor has an EGFR or ALK genomic aberration and had disease progression on or after receiving treatment for the EGFR or ALK aberration(s) prior to receiving the anti-PD-1 antibody, or antigen binding fragment thereof.

In a fourth embodiment (Embodiment E4), the invention comprises a method of treating metastatic or Stage III non-small cell lung cancer (NSCLC) in a human patient in need thereof comprising: (1) subcutaneously administering a dose of from about 600 mg to about 1000 mg of an anti-PD-1 antibody (e.g., pembrolizumab), or antigen binding fragment thereof, to the patient once every approximately six weeks, and (2) administering pemetrexed and platinum chemotherapy (e.g., carboplatin) to the patient. In sub-embodiments of Embodiment E4, the patient was not previously treated with an anti-cancer therapeutic prior to starting the combination treatment regimen with the anti-PD-1 antibody, or antigen binding fragment thereof, pemetrexed and carboplatin.

In certain embodiments of Embodiments E3 and E4 (including sub-embodiments thereof), the patient has nonsquamous non-small cell lung cancer.

In certain embodiments of Embodiments E3 and E4 (including sub-embodiments thereof) the patient is also treated with carboplatin and paclitaxel or nab-paclitaxel. In one embodiment, the carboplatin is administered by intravenous infusion at an AUC of 5-6 mg/ml/min, the paclitaxel is administered by intravenous infusion 200 mg/m² every 21 days, and the nab-paclitaxel is administered by intravenous infusion 100 mg/m² every 7 days.

In sub-embodiments of Embodiment E4, pemetrexed is administered to the patient in an amount of 500 mg/m².

In sub-embodiments of Embodiment E4, pemetrexed is administered to the patient in an amount of 500 mg/m² every 3 weeks.

In sub-embodiments of Embodiment E4, pemetrexed is administered to the patient via intravenous infusion every 21 days. In specific embodiments, the infusion time is about 10 minutes.

In sub-embodiments of Embodiment E4 (Embodiment E4-A), the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally.

In sub-embodiments of Embodiments E4 and E4-A (Embodiment E4-B), the invention further comprises administering about 1 mg of vitamin B₁₂ to the patient about 1 week prior to the first administration of pemetrexed and about every three cycles of pemetrexed administration (i.e., approximately every 9 weeks). In certain embodiments, the vitamin B₁₂ is administered intramuscularly.

In sub-embodiments of Embodiments E4, E4-A and E4-B (Embodiment E4-C), the invention further comprises administering about 4 mg of dexamethasone to the patient twice a day on the day before, the day of, and the day after pemetrexed administration. In certain embodiments the dexamethasone is administered orally. In a fifth embodiment (Embodiment E5), the invention comprises a method of treating recurrent or metastatic head and neck squamous cell cancer (HNSCC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In sub-embodiments of Embodiment E5 (Embodiment E5-A), the patient was previously treated with platinum-containing chemotherapy. In certain embodiments, the patient had disease progression on or after platinum-containing chemotherapy.

In sub-embodiments of Embodiment E5 (Embodiment E5-B), the patient has metastatic or unresectable, recurrent HNSCC and the method further comprises administering platinum and 5-FU (Fluorouracil) for first-line treatment of the HNSCC.

In sub-embodiments of Embodiments E5 (Embodiment E5-C), the anti-PD-1 antibody (e.g., pembrolizumab) is administered as a single agent for the first line treatment of a patient with metastatic or unresectable, recurrent HNSCC, wherein the patient's tumors express PD-L1 (CPS ≥1%).

In sub-embodiments of Embodiments E5 (Embodiment E5-D), the anti-PD-1 antibody (e.g., pembrolizumab) is administered in combination with platinum and 5-fluorouracil (5-FU) chemotherapy, in patients for the first-line treatment of metastatic or unresectable recurrent head and neck squamous cell carcinoma whose tumours express PD-L1 with a CPS ≥1.

In sub-embodiments of Embodiments E5 (Embodiment E5-E), the anti-PD-1 antibody (e.g., pembrolizumab) is administered for the treatment of recurrent or metastatic head and neck squamous cell carcinoma in patients whose tumours express PD-L1 with a ≥50% TPS and progressing on or after platinum-containing chemotherapy.

In embodiments of Embodiments E5-B and E5-D, the platinum therapy is carboplatin administered by intravenous infusion at an AUC of 5 mg/ml/min every three weeks, or cisplatin administered by intravenous infusion 100 mg/m² every three weeks, and 5-FU is administered 1000 mg/m²/day 4 days continuous every three weeks.

In a sixth embodiment (Embodiment E6), the invention comprises a method of treating refractory or relapsed classical Hodgkin lymphoma (cHL) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a seventh embodiment (Embodiment E7), the invention comprises a method of treating classical Hodgkin lymphoma (cHL) in a human patient in need thereof comprising subcutaneously administering a dose of from about 600 mg to about 1000 mg of an anti-PD-1 antibody (e.g., pembrolizumab), or antigen binding fragment thereof, to the patient once every approximately six weeks, wherein the patient has relapsed after (a) one or more lines of therapy for cHL, (b) 2 or more lines of therapy for cHL, or (c) 3 or more lines of therapy for cHL.

In sub-embodiments of Embodiments E6 and E7, the patient is an adult patient.

In alternative sub-embodiments of Embodiments E6 and E7, the patient is a pediatric patient.

In an eighth embodiment (Embodiment E8), the invention comprises a method of treating locally advanced or metastatic urothelial carcinoma in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In one embodiment, the patient is not eligible for platinum-containing chemotherapy or has disease progression during or following platinum-containing chemotherapy or within 12 months of neoadjuvant or adjuvant treatment with platinum-containing chemotherapy.

In sub-embodiments of Embodiment E8, the patient is not eligible for cisplatin-containing chemotherapy.

In sub-embodiments of Embodiment E8, the patient had disease progression during or following platinum-containing chemotherapy or within 12 months of neoadjuvant or adjuvant treatment with platinum-containing chemotherapy.

In sub-embodiments of Embodiment E8, the patient's tumor expresses PD-L1. In other sub-embodiments of Embodiment E8, the patient tumor expresses PD-L1 (CPS ≥10).

In a ninth embodiment (Embodiment E9), the invention comprises a method of treating unresectable or metastatic, microsatellite instability-high (MSI-H) or mismatch repair (MMR) deficient solid tumors in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a sub-embodiment of Embodiment E9, the patient had disease progression following prior anti-cancer treatment.

In a sub-embodiment of Embodiment E9, the patient has advanced or recurrent endometrial carcinoma. In one embodiment, there is disease progression on or following prior treatment with a platinum-containing therapy and the patient is not a candidate for curative surgery or radiation.

In a sub-embodiment of Embodiment E9, the patient has unresectable or metastatic gastric, small intestine, or biliary cancer. In one embodiment, the patient has disease progression on or following at least one prior therapy.

In a tenth embodiment (Embodiment E10), the invention comprises a method of treating unresectable or metastatic, MSI-H or MMR deficient colorectal cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a sub-embodiment of Embodiment E10, the patient had disease progression following prior treatment with a fluoropyrimidine, oxaliplatin, and irinotecan.

In an eleventh embodiment (Embodiment E11), the invention comprises a method of treating recurrent locally advanced unresectable or metastatic gastric cancer or gastroesophageal junction adenocarcinoma in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In specific embodiments, the gastric or gastroesophageal junction adenocarcinoma is HER2-positive. In specific embodiments, the invention further comprises treating the patient with trastuzumab, fluoropyrimidine and platinum-containing chemotherapy. In specific embodiments, the treatment with the anti-PD-1 antibody, trastuzumab, fluoropyrimidine and platinum-containing chemotherapy is a first-line treatment. In one embodiment, the trastuzumab is administered at 8 mg/kg on first infusion and 6 mg/kg in subsequent cycles, followed by chemotherapy: cisplatin 80 mg/m² for up to 6 cycles and 5-FU 800 mg/m²/day for 5 days or oxaliplatin 130 mg/m² up to 6-8 cycles, each administered every three weeks and capecitabine 1,000 mg/m² twice a day for 14 days. In one embodiment, the anti-PD-1 antibody (e.g. pembrolizumab) administered subcutaneously every 6 weeks is administered prior to trastuzumab and chemotherapy on Day 1 of each cycle.

In a twelfth embodiment (Embodiment E12), the invention comprises a method of treating recurrent locally advanced or metastatic esophageal or gastroesophageal junction adenocarcinoma in a human patient in need thereof comprising subcutaneously administering a dose of from about 600 mg to about 1000 mg an anti-PD-1 antibody (e.g., pembrolizumab), or antigen binding fragment thereof, to the patient once approximately every six weeks.

In sub-embodiments of Embodiments E12, the method further comprises administering in combination fluoropyrimidine-based chemotherapy and platinum-containing chemotherapy.

In another twelfth embodiment, (Embodiment E12), the invention comprises a method of treating recurrent locally advanced or metastatic esophagus or HER-2 negative gastroesophageal junction adenocarcinoma in a human patient in need thereof comprising subcutaneously administering a dose of from about 600 mg to about 1000 mg an anti-PD-1 antibody (e.g., pembrolizumab), or antigen binding fragment thereof, to the patient once approximately every six weeks, in combination with fluoropyrimidine-based chemotherapy and platinum-containing chemotherapy, and the patient's tumor has a PD-L1 Combined Positive Score (CPS) ≥10.

In sub-embodiments of Embodiments E12, the fluoropyrimidine-based chemotherapy and platinum-containing chemotherapy is cisplatin 80 mg/m² for up to 6 cycles and 5-FU 800 mg/m²/day for 5 days or oxaliplatin 130 mg/m² up to 6-8 cycles, each administered every three weeks and capecitabine 1,000 mg/m² twice a day for 14 days.

In sub-embodiments of Embodiments E11 and E12, the patient's tumor expresses PD-L1.

In sub-embodiments of Embodiments E11 and E12, the patient's tumor has a PD-L1 Combined Positive Score (CPS) ≥1.

In sub-embodiments of Embodiments E11 and E12, the patient had disease progression on or after one or more prior lines of therapy. In specific embodiments, the prior lines of therapy include fluoropyrimidine and platinum-containing chemotherapy.

In sub-embodiments of Embodiments E11 and E12, the patient had disease progression on or after two or more prior lines of therapy including fluoropyrimidine- and platinum-containing chemotherapy.

In sub-embodiments of Embodiments E11 and E12, the patient had disease progression on or after one or more prior lines of therapy including HER2/neu-targeted therapy.

In sub-embodiments of Embodiments E11 and E12, the patient had disease progression on or after two or more prior lines of therapy including HER2/neu-targeted therapy.

In a thirteenth embodiment (Embodiment E13), the invention comprises a method of treating cancer in a human patient in need thereof comprising subcutaneously administering a dose of from about 600 mg to about 1000 mg of an anti-PD-1 antibody (e.g. pembrolizumab), or antigen binding fragment thereof, to the patient once every approximately six weeks, wherein the patient has a cancer selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, hepatocellular carcinoma, merkel cell carcinoma renal cell carcinoma, endometrial carcinoma, cutaneous squamous cell carcinoma, thyroid cancer, and salivary cancer.

In a fourteenth embodiment (Embodiment E14), the invention comprises a method of treating cancer in a human patient in need thereof comprising subcutaneously administering a dose of from about 600 mg to about 1000 mg of an anti-PD-1 antibody (e.g. pembrolizumab), or antigen binding fragment thereof, to the patient once approximately every six weeks, wherein the patient has small cell lung cancer. In a sub-embodiment, the patient was previously treated with platinum-based chemotherapy and at least one other prior line of therapy.

In a fifteenth embodiment (Embodiment E15), the invention comprises a method of treating non-Hodgkin lymphoma in a human patient in need thereof comprising subcutaneously administering a dose of from about 600 mg to about 1000 mg of an anti-PD-1 antibody (e.g. pembrolizumab), or antigen binding fragment thereof, to the patient once approximately every six weeks.

In a sub-embodiment of Embodiment E15, the non-Hodgkin lymphoma is primary mediastinal large B-cell lymphoma (PMBCL). In specific embodiments where the patient has PMBCL, the patient has refractory PMBCL. In specific embodiments, the patient has relapsed after one or more prior lines of therapy. In specific embodiments, the patient has relapsed after two or more prior lines of therapy. In specific embodiments, the patient was not previously treated with another line of therapy. In specific embodiments, the patient is an adult. In specific embodiments, the patient is a pediatric patient.

In a sixteenth embodiment (Embodiment E16), the invention comprises a method of treating metastatic squamous NSCLC in a human patient in need thereof comprising: (1) subcutaneously administering a dose of from about 600 mg to about 1000 mg of an anti-PD-1 antibody (e.g., pembrolizumab), or antigen binding fragment thereof, to the patient once approximately every six weeks, and (2) administering (i) carboplatin and paclitaxel, or (ii) carboplatin and nab-paclitaxel to the patient.

In a seventeenth embodiment (Embodiment E17), the invention comprises a method of treating Merkel cell carcinoma (MCC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In particular, sub-embodiments of Embodiment E17, the cancer is recurrent, locally advanced MCC. In particular, sub-embodiments of Embodiment E17, the cancer is metastatic MCC.

In sub-embodiments of Embodiment E17, the patient is an adult patient. In alternative sub-embodiments of Embodiment E17, the patient is a pediatric patient.

In a eighteenth embodiment (Embodiment E18), the invention comprises a method for adjuvant therapy of melanoma in a human patient in need thereof comprising subcutaneously administering a dose of from about 600 mg to about 1000 mg of an anti-PD-1 antibody (e.g., pembrolizumab), or antigen binding fragment thereof, to a patient once every approximately six weeks, wherein the patient has previously had one or more melanoma lesions resected. In sub-embodiments of Embodiment E18, the method comprises treating resected high-risk stage III melanoma. In sub-embodiments of Embodiment E18, the method comprises treating resected stage IIB or IIC melanoma.

In a nineteenth embodiment (Embodiment E19), the invention comprises a method of treating hepatocellular carcinoma (HCC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks. In specific embodiments of Embodiment E19, the patient was previously treated with sorafenib.

In a twentieth embodiment (Embodiment E20), the invention comprises a method of treating renal cell carcinoma (RCC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In sub-embodiments of Embodiment E20, the cancer is advanced clear cell RCC.

In sub-embodiments of Embodiment E20, the patient has advanced or metastatic renal cell carcinoma (RCC).

In sub-embodiments of Embodiment E20 (Embodiment E20C), the anti-PD-1 antibody (e.g. pembrolizumab) is for the adjuvant treatment of patients with RCC at intermediate-high or high risk of recurrence following nephrectomy, or following nephrectomy and resection of metastatic lesions.

In a twenty-first embodiment (Embodiment E21), the invention comprises a method of treating breast cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a sub-embodiment of Embodiment E21, the breast cancer is triple negative breast cancer. In a further sub-embodiment, the patient is further treated with chemotherapy. In a further sub-embodiment, the TNBC is recurrent unresectable or metastatic TNBC and the patient's tumors express PD-L1 (CPS ≥10).

In a sub-embodiment of Embodiment E21, the breast cancer is ER+/HER2− breast cancer.

In a further sub-embodiment of Embodiment E21, the patient has high-risk early stage or locally advanced TNBC and the method comprises treating the patient with the anti-PD-1 antibody (e.g. pembrolizumab) in combination with chemotherapy as neoadjuvant treatment, and then treating the patient with the anti-PD-1 antibody (e.g. pembrolizumab) as a single agent as adjuvant treatment after surgery. In one embodiment, the patient is administered four cycles of neoadjuvant anti-PD-1 antibody (e.g. pembrolizumab) on Day 1 of cycles 1-4 of treatment regimen in combination with: Carboplatin at AUC 5 mg/mL/min every 3 weeks on Day 1 of cycles 1-4 of the treatment regimen or AUC 1.5 mg/mL/min every week on Day 1, 8, and 15 of cycles 1-4 of the treatment regimen and Paclitaxel 80 mg/m² every week on Day 1, 8, and 15 of cycles 1-4 of treatment regimen; followed by four additional cycles of neoadjuvant anti-PD-1 antibody (e.g. pembrolizumab) on Day 1 of cycles 5-8 of the treatment regimen in combination with Doxorubicin 60 mg/m² or epirubicin 90 mg/m² every 3 weeks on Day 1 of cycles 5-8 of the treatment regimen and Cyclophosphamide 600 mg/m² every 3 weeks on Day 1 of cycles 5-8 of the treatment regimen; and Following surgery, 9 cycles of adjuvant anti-PD-1 antibody (e.g. pembrolizumab).

In a twenty-second embodiment (Embodiment E22), the invention comprises a method of treating nasopharyngeal cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a twenty-third embodiment (Embodiment E23), the invention comprises a method of treating thyroid cancer in a human patient in need thereof comprising administering a dose of from about 600 mg to about 1000 mg of an anti-PD-1 antibody (e.g., pembrolizumab), or antigen binding fragment thereof, to the patient once approximately every six weeks.

In a twenty-fourth embodiment (Embodiment E24), the invention comprises a method of treating salivary cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In a twenty-fifth embodiment (Embodiment E25), the invention comprises a method of treating cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks, wherein the cancer is selected from the group consisting of: melanoma, non-small cell lung cancer (NSCLC), head and neck squamous cell cancer (HNSCC), relapsed or refractory classical Hodgkin lymphoma (cHL), primary mediastinal large B-cell lymphoma (PMBCL), urothelial carcinoma, microsatellite instability-high or mismatch repair deficient colorectal cancer, gastric cancer, esophageal cancer, cervical cancer, hepatocellular carcinoma, merkel cell carcinoma, renal cell carcinoma, endometrial carcinoma, TMB-H cancer, cutaneous squamous cell carcinoma, and triple-negative breast cancer.

In a sub-embodiment of Embodiment 25 (Embodiment 25B), the invention comprises a method of treating cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks, wherein the cancer is selected from the group consisting of: melanoma, non-small cell lung cancer, relapsed or refractory classical Hodgkin lymphoma, primary mediastinal large B-cell lymphoma, head and neck squamous cell cancer, urothelial carcinoma, esophageal cancer, gastric cancer, cervical cancer, PMBCL, MSI-H cancer, hepatocellular carcinoma, Merkel cell carcinoma, renal cell carcinoma, endometrial carcinoma, TMB-H cancer, cutaneous squamous cell carcinoma, and triple-negative breast cancer.

In a twenty-sixth embodiment (Embodiment E26), the invention comprises a method of treating cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks, wherein the cancer is a Heme malignancy.

In a sub-embodiment of Embodiment E26, the heme malignancy is selected from the group consisting of: acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (MCL-1), myelodysplastic syndrome (MDS), non-Hodgkin lymphoma (NHL), and small lymphocytic lymphoma (SLL).

In a twenty-seventh embodiment (Embodiment E27), the invention comprises a method of treating cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks, wherein the patient has a tumor with a high mutational burden. In sub-embodiments of Embodiment E27, the tumor is a solid tumor. In specific embodiments, the patient is an adult patient. In specific embodiments, the patient is a pediatric patient.

In sub-embodiments of Embodiment 27, a high mutational burden is at least about 10 mutations per megabase of genome examined. In other embodiments, the high mutational burden is at least about 11 mutations per megabase of genome examined, at least about 12 mutations per megabase of genome examined, or at least about 13 mutations per megabase of genome examined.

In a twenty-eighth embodiment (Embodiment E28), the invention comprises a method of treating esophageal cancer in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks.

In sub-embodiments of Embodiment E28, the patient progressed with one previous line of standard therapy prior to receiving the anti-PD-1 antibody, or antigen binding fragment thereof. In a further embodiment, the patient progressed with one or more lines of standard therapy prior to receiving the anti-PD-1 antibody, or antigen binding fragment thereof. In another embodiment, the patient progressed with two or more lines of standard therapy prior to receiving the anti-PD-1 antibody, or antigen binding fragment thereof. In particular embodiments, the standard therapy includes one or more of: paclitaxel, docetaxel, or irinotecan.

In sub-embodiments of Embodiment E28, the patient has advanced or metastatic adenocarcinoma or squamous cell carcinoma of the esophagus.

In sub-embodiments of Embodiment E28, the patient has advanced or metastatic Siewert type I adenocarcinoma of the esophagogastric junction.

In sub-embodiments of Embodiment E28, the patient's tumor expresses PD-L1 (Combined Positive Score [CPS]≥10).

In a twenty-ninth embodiment (Embodiment E29), the invention comprises a method of treating high-risk non-muscle invasive bladder cancer (NMIBC) in a human patient in need thereof comprising subcutaneously administering a dose of from about 600 mg to about 1000 mg of an anti-PD-1 antibody (e.g., pembrolizumab), or antigen binding fragment thereof, to the patient once every approximately six weeks. In specific embodiments, the patient has NMIBC with carcinoma in situ (CIS) or CIS plus papillary disease.

In a sub-embodiment of Embodiment E29, the patient was previously treated with standard therapy prior to being treated with the anti-PD-1 antibody, or antigen binding fragment thereof. In specific embodiments, the prior therapy is Bacillus Calmette-Guerin (BCG) therapy. In particular embodiments, the patient did not respond to BCG therapy. In specific embodiments, the patient was ineligible for radical cystectomy or chose not to undergo radical cystectomy.

In a thirtieth embodiment (Embodiment E30), the invention comprises a method of treating cutaneous squamous cell carcinoma (cSCC) in a human patient in need thereof comprising subcutaneously administering a dose of from about 600 mg to about 1000 mg of an anti-PD-1 antibody (e.g., pembrolizumab), or antigen binding fragment thereof, to the patient once every approximately six weeks. In specific embodiments, the cutaneous squamous cell carcinoma is not curable by surgery or radiation.

In a thirty-first embodiment (Embodiment E31), the invention comprises a method of treating endometrial carcinoma in a human patient in need thereof comprising subcutaneously administering a dose of from about 600 mg to about 1000 mg of an anti-PD-1 antibody (e.g., pembrolizumab), or antigen binding fragment thereof, to the patient once every approximately six weeks.

In some sub-embodiments of Embodiment E31, the endometrial carcinoma is advanced endometrial carcinoma that is not MSI-H or mismatch repair deficient (dMMR). In specific embodiments, the patient had disease progression following prior systemic therapy.

In some sub-embodiments of Embodiment E31, the endometrial carcinoma is advanced endometrial carcinoma that is MSI-H or dMMR, as determined by an FDA-approved test, wherein the patient has had disease progression following prior systemic therapy in any setting. In specific embodiments, the patient is not a candidate for curative surgery or radiation.

In a thirty-second embodiment (Embodiment E32), the invention comprises a method of treating cervical carcinoma in a human patient in need thereof comprising subcutaneously administering a dose of from about 600 mg to about 1000 mg of an anti-PD-1 antibody (e.g., pembrolizumab), or antigen binding fragment thereof, to the patient once every approximately six weeks. In one embodiment, the cervical cancer is recurrent or metastatic cervical cancer and the patient had disease progression on or after chemotherapy.

In sub-embodiments of Embodiment E32, the method further comprises treating the patient with chemotherapy, with or without bevacizumab. In specific embodiments, the cervical cancer is persistent, recurrent, or metastatic cervical cancer and the patient's tumor expresses PD-L1 (CPS≥1). In one embodiment, the chemotherapy is paclitaxel 175 mg/m² and cisplatin 50 mg/m² or paclitaxel 175 mg/m² and carboplatin AUC 5 mg/mL/min, and administered on Day 1 every three weeks. In one embodiment, the chemotherapy with bevacizumab is paclitaxel 175 mg/m² and cisplatin 50 mg/m² or paclitaxel 175 mg/m² and carboplatin AUC 5 mg/mL/min and bevacizumab 15 mg/kg, and administered on Day 1 every three weeks.

In sub-embodiments of Embodiment E32, the cervical cancer is recurrent or metastatic cervical cancer with disease progression on or after chemotherapy, the patient's tumor expresses PD-L1 (CPS≥1).

In a thirty-third embodiment (Embodiment E33), the invention comprises a method of treating Stage IB, II, or IIIA non-small cell lung cancer (NSCLC) in a human patient in need thereof comprising subcutaneously administering a dose of from about 600 mg to about 1000 mg of an anti-PD-1 antibody (e.g., pembrolizumab), or antigen binding fragment thereof, to the patient once approximately every six weeks for adjuvant treatment following resection.

In sub-embodiments of Embodiment E33 (E33-A), the patient had prior platinum-based chemotherapy.

In any of the methods or uses of the invention described herein (including Embodiments E1-E33), the anti-PD-1 antibody, or antigen binding fragment thereof, is any of the antibodies or antigen-binding fragments described in Section II of the Detailed Description of the Invention “PD-1 Antibodies and Antigen Binding Fragments Useful in the Invention” herein. In specific embodiments, the anti-PD-1 antibody is pembrolizumab, or an antigen-binding fragment thereof, or an antibody which cross competes with pembrolizumab for binding to human PD-1. In specific embodiments, the anti-PD-1 antibody is a pembrolizumab variant.

In any of the methods or uses of the invention described herein (including Embodiments E1-E33), the anti-PD-1 antibody, or antigen binding fragment thereof, can be co-administered or co-formulated with a hyaluronidase described in Section III. In one embodiment, the anti-PD-1 antibody, or antigen binding fragment thereof is co-administered with a human hyaluronidase. In another embodiment, the anti-PD-1 antibody, or antigen binding fragment thereof, is co-formulated with a human hyaluronidase. In specific embodiments, the human hyaluronidase is rHuPH20. In specific embodiments, the human hyaluronidase is PH20 variant 2.

In any one of the methods or uses of the invention described herein (including Embodiments E1-E33), the anti-PD-1 antibody or antigen-binding fragment thereof is administered to the patient at a dose from about 600 mg to about 1000 mg every six weeks. In further embodiments, the anti-PD-1 antibody or antigen-binding fragment thereof administered to the patient is at a dose from about 600 mg to about 950 mg every six weeks. In further embodiments, the anti-PD-1 antibody or antigen-binding fragment is administered at a dose from about 600 mg to about 900 mg every six weeks. In further embodiments, the anti-PD-1 antibody or antigen-binding fragment thereof administered to the patient is at a dose from about 650 mg to about 800 mg every six weeks. In further embodiments, the anti-PD-1 antibody or antigen-binding fragment thereof administered to the patient is at a dose from about 660 mg to about 800 mg every six weeks. In further embodiments, the anti-PD-1 antibody or antigen-binding fragment is administered at a dose from about 700 mg to about 800 mg every six weeks. In further embodiments, the anti-PD-1 antibody or antigen-binding fragment is administered at a dose from about 760 mg to about 800 mg every six weeks. In further embodiments, the anti-PD-1 antibody or antigen-binding fragment is administered at a dose from about 760 mg to about 790 mg every six weeks.

In any one of the methods or uses of the invention described herein (including Embodiments E1-E33), the anti-PD-1 antibody or antigen-binding fragment is administered at a dose from 600 mg to 1000 mg every six weeks. In further embodiments, the anti-PD-1 antibody or antigen-binding fragment is administered to the patient at a dose from 600 mg to 950 mg every six weeks. In further embodiments, the anti-PD-1 antibody or antigen-binding fragment is administered at a dose from 600 mg to 900 mg every six weeks. In further embodiments, the anti-PD-1 antibody or antigen-binding fragment thereof administered to the patient is at a dose from 650 mg to 800 mg every six weeks. In further embodiments, the anti-PD-1 antibody or antigen-binding fragment thereof administered to the patient is at a dose from 660 mg to 800 mg every six weeks. In further embodiments, the anti-PD-1 antibody or antigen-binding fragment is administered at a dose from 700 mg to 800 mg every six weeks. In further embodiments, the anti-PD-1 antibody or antigen-binding fragment is administered at a dose from 760 mg to 800 mg every six weeks. In further embodiments, the anti-PD-1 antibody or antigen-binding fragment is administered at a dose from 760 mg to 790 mg every six weeks.

In any one of the methods or uses of the invention described herein (including Embodiments E1-E33), the anti-PD-1 antibody or antigen binding fragment thereof is administered at a dose of 760 mg every six weeks. In one embodiment, the anti-PD-1 antibody or antigen-binding fragment is administered at a dose of 770 mg every six weeks. In another embodiment, the anti-PD-1 antibody or antigen-binding fragment is administered at a dose of 780 mg every six weeks. In another embodiment, the anti-PD-1 antibody or antigen-binding fragment is administered at a dose of 790 mg every six weeks. In another embodiment, the anti-PD-1 antibody or antigen-binding fragment is administered at a dose of 800 mg every six weeks. In another embodiment, the anti-PD-1 antibody or antigen-binding fragment is administered at a dose of 810 mg every six weeks. In another embodiment, the anti-PD-1 antibody or antigen-binding fragment is administered at a dose of 650 mg every six weeks. In another embodiment, the anti-PD-1 antibody or antigen-binding fragment is administered at a dose of 750 mg every six weeks.

In any of the methods or uses of the invention described herein (including Embodiments E1-E33), the anti-PD-1 antibody, or antigen binding fragment thereof, is subcutaneously administered to the patient at a dose of from about 300 mg to about 500 mg approximately every three weeks with a human hyaluronidase (including those described under Section III). In any of the methods or uses of the invention described herein (including Embodiments E1-E33), the anti-PD-1 antibody, or antigen binding fragment thereof, is subcutaneously administered to the patient at a dose of from about 300 mg to about 500 mg approximately every three weeks with PH20 variant 2. In any of the methods or uses of the invention described herein (including Embodiments E1-E33), the anti-PD-1 antibody, or antigen binding fragment thereof, is subcutaneously administered to the patient at a dose of from about 300 mg to about 500 mg approximately every three weeks with rHuPH20. In particular embodiments, the anti-PD-1 antibody, or antigen binding fragment thereof, is subcutaneously administered to the patient every three weeks, every three weeks ±5 days, ±4 days, ±3 days, ±2 days or ±1 day.

In further embodiments, the anti-PD-1 antibody or antigen-binding fragment is administered at a dose of from about 300 mg to about 500 mg every three weeks with the human hyaluronidase. In further embodiments, the anti-PD-1 antibody or antigen-binding fragment is administered at a dose of from about 350 mg to about 450 mg every three weeks with the human hyaluronidase. In further embodiments, the anti-PD-1 antibody or antigen-binding fragment is administered at a dose of from about 360 mg to about 420 mg every three weeks with the human hyaluronidase. In further embodiments, the anti-PD-1 antibody or antigen-binding fragment is administered at a dose of from about 380 mg to about 410 mg every three weeks with the human hyaluronidase. In further embodiments, the anti-PD-1 antibody or antigen-binding fragment is administered at a dose of 380 mg every three weeks with the human hyaluronidase. In further embodiments, the anti-PD-1 antibody or antigen-binding fragment is administered at a dose of 385 mg every three weeks with the human hyaluronidase. In another embodiment, the anti-PD-1 antibody or antigen-binding fragment is administered at a dose of 390 mg every three weeks with the human hyaluronidase. In another embodiment, the anti-PD-1 antibody or antigen-binding fragment is administered at a dose of 395 mg every three weeks with the human hyaluronidase. In another embodiment, the anti-PD-1 antibody or antigen-binding fragment is administered at a dose of 325 mg every three weeks with the human hyaluronidase. In another embodiment, the anti-PD-1 antibody or antigen-binding fragment is administered at a dose of 400 mg every three weeks with the human hyaluronidase.

In further embodiments, the anti-PD-1 antibody or antigen-binding fragment is administered at a dose of from 300 mg to 500 mg every three weeks with the human hyaluronidase. In further embodiments, the anti-PD-1 antibody or antigen-binding fragment is administered at a dose of from 350 mg to 450 mg every three weeks with the human hyaluronidase. In further embodiments, the anti-PD-1 antibody or antigen-binding fragment is administered at a dose of from 360 mg to 420 mg every three weeks with the human hyaluronidase. In further embodiments, the anti-PD-1 antibody or antigen-binding fragment is administered at a dose of from 380 mg to 410 mg every three weeks with the human hyaluronidase.

In any one of the methods or uses of the invention described above (including Embodiments E1-E33), the anti-PD-1 antibody, or antigen binding fragment thereof is co-administered with the human hyaluronidase PH20 variant or fragment defined herein. In another embodiment, the anti-PD-1 antibody, or antigen binding fragment thereof is co-formulated with PH20 variant 2 in a composition. For example, US20220089739, the contents of which are hereby incorporated by reference, describes the preparation of liquid compositions comprising pembrolizumab and PH20 variants.

In one embodiment, the composition comprises 130 mg/ml of the anti-PD-1 antibody or antigen binding fragment thereof. In other embodiments, the composition comprises 165 mg/ml of the anti-PD-1 antibody or antigen binding fragment thereof.

In further embodiments, the composition further comprises L-methionine. In particular embodiments, the L-methionine is present in a concentration of about 10 mM.

In further embodiments, the composition further comprises histidine buffer at about pH 5.0 to pH 6.0. In particular embodiments, the histidine is present in a concentration of about 10 mM.

In further embodiments, the composition further comprises sucrose. In particular embodiments, the sucrose is present in a concentration of about 70 mg/mL. In particular embodiments, the sucrose is present at a concentration of 7% (w/v).

In further of the invention, the composition further comprises polysorbate 80. In particular embodiments, the polysorbate 80 is present in a concentration of about 0.2 mg/mL. In particular embodiments, the polysorbate 80 is present at a concentration of 0.02% (w/v).

In specific embodiments, the composition comprises 10 mM L-methionine, 10 mM histidine, pH 5.5, 7% sucrose, 0.02% polysorbate 80, and 130 mg/mL of the anti-PD-1 antibody or antigen-binding fragment thereof.

In specific embodiments, the composition comprises 10 mM L-methionine, 10 mM histidine, pH 5.5, 7% sucrose, 0.02% polysorbate 80, and 165 mg/mL of the anti-PD-1 antibody or antigen-binding fragment thereof.

In specific embodiments of the methods or uses described herein, the anti-PD-1 antibody, or antigen binding fragment thereof, is administered subcutaneously in one or more injections. In specific embodiments, the anti-PD-1 antibody, or antigen binding fragment thereof, is administered in 2 injections.

In one embodiment, 790 mg of the anti-PD-1 antibody, or antigen binding fragment thereof, is administered subcutaneously as a composition comprising 130 mg/mL in one injection. In one embodiment, 790 mg of the anti-PD-1 antibody, or antigen binding fragment thereof, is administered subcutaneously in two injections.

In one embodiment, 790 mg of the anti-PD-1 antibody, or antigen binding fragment thereof, is administered subcutaneously as a composition comprising 165 mg/mL in one injection. In one embodiment, 790 mg of the anti-PD-1 antibody, or antigen binding fragment thereof, is administered subcutaneously as a composition comprising 165 mg/mL in two injections. In a further embodiment, 4.8 mL of the composition comprising 165 mg/mL of the anti-PD-1 antibody, or antigen binding fragment thereof, is administered subcutaneously in one injection.

In one embodiment, 395 mg of the anti-PD-1 antibody, or antigen binding fragment thereof, is administered subcutaneously as a composition comprising 130 mg/mL in one injection. In one embodiment, 395 mg of the anti-PD-1 antibody, or antigen binding fragment thereof, is administered subcutaneously in two injections.

In one embodiment, 395 mg of the anti-PD-1 antibody, or antigen binding fragment thereof, is administered subcutaneously as a composition comprising 165 mg/mL in one injection. In one embodiment, 395 mg of the anti-PD-1 antibody, or antigen binding fragment thereof, is administered subcutaneously as a composition comprising 165 mg/mL in two injections. In a further embodiment, 2.4 mL of the composition comprising 165 mg/mL of the anti-PD-1 antibody, or antigen binding fragment thereof, is administered subcutaneously in one injection. In a further embodiment, 1.2 mL of the composition comprising 165 mg/mL of the anti-PD-1 antibody, or antigen binding fragment thereof, is administered subcutaneously in each of the two injections.

In any methods and uses of the invention, the dose of the anti-PD-1 antibody, or antigen binding fragment thereof described herein is co-formulated or co-administered with a dose of about 700 Units to about 50000 Units of a human hyaluronidase. In one embodiment, the dose is about 2000 Units to about 20000 Units of a human hyaluronidase. In one embodiment, the dose is about 5000 Units to about 15000 Units of a human hyaluronidase. In one embodiment, about 10000 Units to about 30000 Units human hyaluronidase is co-formulated or co-administered with the dose of the anti-PD-1 antibody, or antigen binding fragment thereof. In one embodiment, the dose is about 7000 Units to about 13000 Units of a human hyaluronidase. In one embodiment, the dose is about 8000 Units to about 10000 Units of a human hyaluronidase. In one embodiment, the dose is about 4000 Units to about 6000 Units of a human hyaluronidase. In one embodiment, the dose is 4800 Units of a human hyaluronidase. In one embodiment, the dose is 9600 Units of a human hyaluronidase.

In one embodiment, the human hyaluronidase is co-formulated or co-administered with the anti-PD-1 antibody, or antigen binding fragment thereof at a ratio of about 12.15 Units:1 mg. In one embodiment, the human hyaluronidase is co-formulated or co-administered with the anti-PD-1 antibody, or antigen binding fragment thereof at a ratio of about 12 Units:1 mg. In one embodiment, the human hyaluronidase is co-formulated or co-administered with the anti-PD-1 antibody, or antigen binding fragment thereof at a ratio of about 10 to 14 Units:1 mg. In one embodiment, the human hyaluronidase is co-formulated or co-administered with the anti-PD-1 antibody, or antigen binding fragment thereof at a ratio of about 8 to 18 Units:1 mg. In one embodiment, the human hyaluronidase is co-formulated or co-administered with the anti-PD-1 antibody, or antigen binding fragment thereof at a ratio of about 6 to 25 Units:1 mg. In one embodiment, the human hyaluronidase is co-formulated or co-administered with the anti-PD-1 antibody, or antigen binding fragment thereof at a ratio of about 3 to 36 Units:1 mg.

In any of the methods or uses described herein, including Embodiment E1-E33, and sub-embodiments thereof, the method may further comprise administering one or more “additional therapeutic agents” (as used herein, “additional therapeutic agent” refers to an additional agent relative to the anti-PD-1 antibody or antigen-binding fragment thereof). The additional therapeutic agent may be, e.g., a chemotherapeutic, a biotherapeutic agent (including but not limited to antibodies to CTLA4, TIGIT, VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in specific embodiments of the methods or uses of the invention, the method further comprises administering an additional therapeutic agent. In particular embodiments, the additional therapeutic agent is an anti-CTLA4 antibody or antigen binding fragment thereof, an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof, an anti-ILT3 antibody, or antigen binding fragment thereof, or an anti-ILT4 antibody, or antigen binding fragment thereof. In one embodiment, the additional therapeutic agent is a Newcastle disease viral vector expressing IL-12. In a further embodiment, the additional therapeutic agent is dinaciclib. In another embodiment, the additional therapeutic agent is navarixin. In a further embodiment, the additional therapeutic agent is vicriviroc.

In a further embodiment, the additional therapeutic agent is an oncolytic virus. In one embodiment, the additional therapeutic agent is Coxsackievirus or CVA21. In one embodiment, the additional therapeutic agent is CAVATAK™.

In yet another embodiment, the additional therapeutic agent is a STING agonist. In a further embodiment, the additional therapeutic agent is an IL-27 antagonist. In one embodiment, the additional therapeutic agent is a PARP inhibitor. In one embodiment, the additional therapeutic agent is a multi-kinase inhibitor. In one embodiment, the additional therapeutic agent is a MEK inhibitor. In one embodiment, the additional therapeutic agent is a 4-1BB agonist. In one embodiment, the additional therapeutic agent is nemtabrutinib. In another embodiment, the additional therapeutic agent is belzutifan.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In specific embodiments which comprise a step of administering an additional therapeutic agent (i.e., in addition to the anti-PD-1 antibody (e.g., pembrolizumab) or antigen-binding fragment thereof), the additional therapeutic agent in the combination therapy may be administered using the same dosage regimen (dose, frequency and duration of treatment) that is typically employed when the agent is used as monotherapy for treating the same cancer. In other embodiments, the patient receives a lower total amount of the additional therapeutic agent in the combination therapy than when that agent is used as monotherapy, e.g., smaller doses, less frequent doses, and/or shorter treatment duration.

Determination of the appropriate dose is made by the clinician, e.g., using parameters or factors known or suspected in the art to affect treatment or predicted to affect treatment. The appropriate dosage (“therapeutically effective amount”) of the agent will depend, for example, on the condition to be treated, the severity and course of the condition, whether the agent is administered for preventive or therapeutic purposes, previous therapy, the patient's clinical history and response to the agent, the type of agent used, and the discretion of the attending physician. Generally, the dose begins with an amount somewhat less than the optimum dose and it is increased by small increments thereafter until the desired or optimum effect is achieved relative to any negative side effects.

The additional therapeutic agent in a combination therapy can be administered orally, intratumorally, or parenterally, including the intravenous, intramuscular, intraperitoneal, subcutaneous, rectal, topical, and transdermal routes of administration. For example, the combination treatment may comprise an anti-PD-1 antibody or antigen binding fragment thereof, and an anti-CTLA antibody or antigen binding fragment thereof, both of which may be administered intravenously or subcutaneously, as well as a chemotherapeutic agent, which may be administered orally.

In specific embodiments, a combination therapy of the invention may be used prior to or following surgery to remove a tumor and may be used prior to, during, or after radiation therapy. A combination therapy of the invention may also be used when a patient's tumor is non-resectable.

In specific embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention may be used to treat a tumor that is large enough to be found by palpation or by imaging techniques well known in the art, such as MRI, ultrasound, or CAT scan. In specific embodiments, a combination therapy of the invention is used to treat an advanced stage tumor having dimensions of at least about 200 mm³′ 300 mm³, 400 mm³, 500 mm³, 750 mm³, or up to 1000 mm³.

In specific embodiments, a combination therapy of the invention is administered to a human patient who has a cancer that expresses PD-L1. In specific embodiments, PD-L1 expression is detected using a diagnostic anti-human PD-L1 antibody, or antigen binding fragment thereof, in an TIC assay on an FFPE or frozen tissue section of a tumor sample removed from the patient. A patient's physician may order a diagnostic test to determine PD-L1 expression in a tumor tissue sample removed from the patient prior to initiation of treatment with the anti-PD-1 antibody, or antigen-binding fragment thereof, but it is envisioned that the physician could order the first or subsequent diagnostic tests at any time after initiation of treatment, such as for example after completion of a treatment cycle.

### V. Kits and Compositions

The invention also relates to a pharmaceutical composition for subcutaneous injection comprising a dose of from about 600 mg to about 1000 mg of an anti-PD-1 antibody or antigen binding fragment thereof, and from about 700 Units to about 50000 Units of a human hyaluronidase. The invention also relates to a pharmaceutical composition for subcutaneous injection comprising a dose of from about 600 mg to about 1000 mg of an anti-PD-1 antibody or antigen binding fragment thereof, and from about 7290 Units to about 12150 Units of a human hyaluronidase. In one embodiment, the pharmaceutical composition for subcutaneous injection comprises a dose of from about 700 mg to about 800 mg of an anti-PD-1 antibody or antigen binding fragment thereof, and from about 700 Units to about 50000 Units of a human hyaluronidase. In one embodiment, the pharmaceutical composition for subcutaneous injection comprises a dose of from about 700 mg to about 800 mg of an anti-PD-1 antibody or antigen binding fragment thereof, and from about 8505 Units to about 9720 Units of a human hyaluronidase. In one embodiment, the pharmaceutical composition for subcutaneous injection comprises a dose of 790 mg of an anti-PD-1 antibody or antigen binding fragment thereof, and 9600 Units of a human hyaluronidase.

The invention also relates to a pharmaceutical composition for subcutaneous injection comprising a dose of from about 600 mg to about 1000 mg of an anti-PD-1 antibody or antigen binding fragment thereof. In one embodiment, the pharmaceutical composition for subcutaneous injection comprises a dose of from about 700 mg to about 800 mg of an anti-PD-1 antibody or antigen binding fragment thereof. In one embodiment, the pharmaceutical composition for subcutaneous injection comprises a dose of from about 760 mg to about 790 mg of an anti-PD-1 antibody or antigen binding fragment thereof. In one embodiment, the pharmaceutical composition for subcutaneous injection comprises a dose of 790 mg of an anti-PD-1 antibody or antigen binding fragment thereof.

The invention also relates to a pharmaceutical composition for subcutaneous injection comprising a dose of from about 300 mg to about 500 mg of an anti-PD-1 antibody or antigen binding fragment thereof, and from about 700 Units to about 50000 Units of a human hyaluronidase. The invention also relates to a pharmaceutical composition for subcutaneous injection comprising a dose of from about 300 mg to about 500 mg of an anti-PD-1 antibody or antigen binding fragment thereof, and from about 3645 Units to about 6075 Units of a human hyaluronidase. In one embodiment, the pharmaceutical composition for subcutaneous injection comprises a dose of from about 350 mg to about 400 mg of an anti-PD-1 antibody or antigen binding fragment thereof, and from about 700 Units to about 50000 Units of a human hyaluronidase. In one embodiment, the pharmaceutical composition for subcutaneous injection comprises a dose of from about 350 mg to about 400 mg of an anti-PD-1 antibody or antigen binding fragment thereof, and from about 4252 Units to 4860 Units of a human hyaluronidase. In one embodiment, the pharmaceutical composition for subcutaneous injection comprises a dose of 395 mg of an anti-PD-1 antibody or antigen binding fragment thereof, and 4800 Units of a human hyaluronidase. In one embodiment, the pharmaceutical composition for subcutaneous injection comprises a dose of 395 mg of pembrolizumab, and about 33 μg of the human hyaluronidase of SEQ ID NO: 18. In another embodiment, the pharmaceutical composition for subcutaneous injection comprises a dose of 790 mg of pembrolizumab, and about 66 μg of the human hyaluronidase of SEQ ID NO: 18. In one embodiment, the pharmaceutical composition for subcutaneous injection comprises a dose of 395 mg of an anti-PD-1 antibody, and about 33 μg of the human hyaluronidase of SEQ ID NO: 18. In another embodiment, the pharmaceutical composition for subcutaneous injection comprises a dose of 790 mg of an anti-PD-1 antibody, and about 66 μg of the human hyaluronidase of SEQ ID NO: 18. In one embodiment of the foregoing embodiments, the anti-PD-1 antibody is a monoclonal antibody comprising a heavy chain consisting of a sequence of amino acids as set forth in SEQ ID NO: 11 and a light chain consisting of a sequence of amino acids as set forth in SEQ ID NO: 5.

In any of the methods, uses and composition described herein, about 700 Units to about 50000 Units human hyaluronidase is co-formulated with the dose of the anti-PD-1 antibody, or antigen binding fragment thereof. In one embodiment, about 3000 Units to about 15000 Units human hyaluronidase is co-formulated with the dose of the anti-PD-1 antibody, or antigen binding fragment thereof. In one embodiment, about 3000 Units to about 7000 Units human hyaluronidase is co-formulated with the dose of the anti-PD-1 antibody, or antigen binding fragment thereof. In one embodiment, about 7000 Units to about 14000 Units human hyaluronidase is co-formulated with the dose of the anti-PD-1 antibody, or antigen binding fragment thereof. In one embodiment, about 10000 Units to about 30000 Units human hyaluronidase is co-formulated with the dose of the anti-PD-1 antibody, or antigen binding fragment thereof. In one embodiment, about 8000 Units to about 10000 Units human hyaluronidase is co-formulated with the dose of the anti-PD-1 antibody, or antigen binding fragment thereof. In one embodiment, about 4000 Units to about 5000 Units human hyaluronidase is co-formulated with the dose of the anti-PD-1 antibody, or antigen binding fragment thereof. In one embodiment, about 9600 Units human hyaluronidase is co-formulated with the dose of the anti-PD-1 antibody, or antigen binding fragment thereof. In one embodiment, about 4800 Units human hyaluronidase is co-formulated with the dose of the anti-PD-1 antibody, or antigen binding fragment thereof.

In one embodiment, the human hyaluronidase is co-formulated with the anti-PD-1 antibody, or antigen binding fragment thereof at a ratio of about 12.15 Units:1 mg. In one embodiment, the human hyaluronidase is co-formulated or co-administered with the anti-PD-1 antibody, or antigen binding fragment thereof at a ratio of about 12 Units:1 mg. In one embodiment, the human hyaluronidase is co-formulated or co-administered with the anti-PD-1 antibody, or antigen binding fragment thereof at a ratio of about 10 to 14 Units:1 mg. In one embodiment, the human hyaluronidase is co-formulated or co-administered with the anti-PD-1 antibody, or antigen binding fragment thereof at a ratio of about 8 to 18 Units:1 mg. In one embodiment, the human hyaluronidase is co-formulated or co-administered with the anti-PD-1 antibody, or antigen binding fragment thereof at a ratio of about 6 to 25 Units:1 mg. In one embodiment, the human hyaluronidase is co-formulated or co-administered with the anti-PD-1 antibody, or antigen binding fragment thereof at a ratio of about 3 to 36 Units:1 mg.

In the foregoing embodiments, the pharmaceutical composition can be contained in a vial, an on-body device, or one or more pre-filled syringes. Any of the pharmaceutical compositions described herein can be used in the methods or uses described in Section IV.

The invention also relates to a kit for treating a patient with cancer, the kit comprising: (a) a composition for subcutaneous injection comprising a dose of from about 600 mg to about 1000 mg, or from about 300 mg to about 500 mg (or a dose or composition described in Section IV or V), of an anti-PD-1 antibody or antigen binding fragment thereof, and optionally a human hyaluronidase (in any of the amounts described in Section III, IV or V), or any of the pharmaceutical compositions in the foregoing embodiments under this section and (b) instructions for using the composition or pharmaceutical compositions of (a) in any of the methods or uses for treating cancer described herein.

The kits of the invention may provide the anti-PD-1 antibody, or antigen-binding fragment thereof and optionally a human hyaluronidase, in a container and includes a package insert. The container contains at least a dose of from about 600 mg to about 1000 mg, or from about 300 mg to about 500 mg of a composition comprising an anti-PD-1 antibody, or antigen binding fragment thereof, and optionally, about 700 Units to about 50000 Units of a human hyaluronidase, and the package insert, or label, which comprises instructions for treating a patient with cancer using the composition. The container may be comprised of any shape and/or material (e.g., plastic or glass). For example, the container might be a vial, syringe or bottle. The kit may further comprise other materials that may be useful in administering the medicaments, such as on-body devices, needles and syringes. In specific embodiments of the kit, the instructions state that the medicament is intended for use in treating a patient as described in any of Embodiments E1-E33 above in Section IV entitled Methods and Uses of the Invention.

In one embodiment, the composition comprises 130 mg/ml of the anti-PD-1 antibody or antigen binding fragment thereof. In other embodiments, the composition comprises 165 mg/ml of the anti-PD-1 antibody or antigen binding fragment thereof.

In further embodiments, the composition further comprises L-methionine. In particular embodiments, the L-methionine is present in a concentration of about 10 mM.

In further embodiments, the composition further comprises histidine buffer at about pH 5.0 to pH 6.0. In particular embodiments, the histidine is present in a concentration of about 10 mM.

In further embodiments, the composition further comprises sucrose. In particular embodiments, the sucrose is present in a concentration of about 70 mg/mL. In particular embodiments, the sucrose is present at a concentration of 7% (w/v).

In further of the invention, the composition further comprises polysorbate 80. In particular embodiments, the polysorbate 80 is present in a concentration of about 0.2 mg/mL. In particular embodiments, the polysorbate 80 is present at a concentration of 0.02% (w/v).

In specific embodiments, the composition comprises 10 mM L-methionine, 10 mM histidine, pH 5.5, 7% sucrose, 0.02% polysorbate 80, and 130 mg/mL of the anti-PD-1 antibody or antigen-binding fragment thereof.

In specific embodiments, the composition comprises 10 mM L-methionine, 10 mM histidine, pH 5.5, 7% sucrose, 0.02% polysorbate 80, and 165 mg/mL of the anti-PD-1 antibody or antigen-binding fragment thereof.

In further embodiments, the composition further comprises 2000 U/ml of PH20 variant 2.

In further embodiments, the composition further comprises 2000 U/ml of rHuPH20.

In one embodiment, the composition is contained in a vial. In another embodiment, the composition is contained in one or more pre-filled syringes. In one embodiment, the composition is contained in two pre-filled syringes.

In any of the kits or compositions of the invention, the anti-PD-1 antibody or antigen binding fragment can be any of the antibodies or antigen-binding fragments described in Section II of the Detailed Description of the Invention “PD-1 Antibodies and Antigen Binding Fragments Useful in the Invention”. In one embodiment, the anti-PD-1 antibody, or antigen binding fragment thereof, is pembrolizumab. In another embodiment, the anti-PD-1 antibody, or antigen binding fragment thereof, is a pembrolizumab variant.

In any of the kits or compositions of the invention, the anti-PD-1 antibody, or antigen binding fragment thereof, can be co-formulated with a human hyaluronidase described in Section III.

These and other aspects of the invention, including the exemplary specific embodiments listed below, will be apparent from the teachings contained herein.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Population PK Model Development

Pembrolizumab is currently approved for use in multiple cancer indications at a dose of either 200 mg or 2 mg/kg Q3W or 400 mg Q6W administered as an IV infusion. An alternative subcutaneous (“SC”) formulation with hyaluronidase would provide convenience and flexibility to patients and prescribers. A Phase I clinical study, designed to estimate the bioavailability of a SC formulation of pembrolizumab combined with the hyaluronidase PH20 variant 2 (“SC pembrolizumab-HLN”, or “pembrolizumab-HLN SC”) at two different solution strengths of pembrolizumab (concentration 165 mg/ml (Arm 1) and 130 mg/ml (Arm 2)) was performed. The bioavailability of two different subcutaneous solution strengths of pembrolizumab (in two independent study arms) was estimated in this study using the subcutaneous dose (650 mg over a 6-wk dosing interval (cycle), given at two different concentration/volumes (as shown in Table 6) of pembrolizumab compared with the IV dose of pembrolizumab (400 mg over a 6-wk dosing interval)).

TABLE 6
Pembrolizumab-HLN SC Formulations

| | |
|---|---|
| 130 mg/mL pembrolizumab | 165 mg/ml pembrolizumab |
| 2000 U/ml PH20 Variant 2 | 2000 U/ml PH20 Variant 2 |
| 7% w/v sucrose | 7% w/v sucrose |
| 0.02% w/v polysorbate 80 | 0.02% w/v polysorbate 80 |
| 10 mM histidine buffer | 10 mM histidine buffer |
| 10 mM L-methionine | 10 mM L-methionine |

Patients in each study arm with advanced melanoma or non-small cell lung cancer (NSCLC) or Renal Cell Carcinoma (RCC) were each administered both the SC and IV pembrolizumab in a cross-over design as follows: Cycle 1: SC 650 mg; Cycle 2: IV 400 mg; Cycle 3: SC 650 mg; Cycle 4-18: IV 400 mg.

Eligible patients were ≥18 years of age, had unresectable Stage III or IV melanoma not amenable to local therapy, measurable disease per RECIST v1.1, an Eastern Cooperative Oncology Group performance status of 0 or 1, or had not received prior therapy for advanced disease (except BRAF/MEK inhibitor for BRAF^{V600} mutant disease and prior adjuvant or neoadjuvant therapy received ≥4 weeks from randomization); had histologically- or cytologically-confirmed diagnosis of NSCLC that is either Metastatic (Stage IV [M1a, M1b, or M1c per current AJCC criteria]) nonsquamous NSCLC with no EGFR, ALK, or ROS1 genomic tumor aberrations, or Metastatic (Stage IV [M1a, M1b, or M1c per current AJCC criteria]) squamous NSCLC; had histologically or cytologically-confirmed diagnosis of RCC with clear cell component that is with or without sarcomatoid features, unresectable, locally advanced/metastatic (i.e., Stage IV RCC per AJCC); and had not received prior systemic therapy for advanced ccRCC.

For participants with NSCLC or RCC, treatment with pembrolizumab-HLN SC and pembrolizumab IV included combinations with standard of care therapy as follows:

NSCLC: With pemetrexed and platinum (carboplatin or cisplatin) chemotherapy for 1L treatment of patients with metastatic nonsquamous NSCLC with no EGFR or ALK genomic tumor aberrations; or with carboplatin and either paclitaxel or nab-paclitaxel for 1L treatment of patients with metastatic squamous NSCLC.

RCC: With axitinib for 1L treatment of patients with advanced RCC.

TABLE 7
Study Interventions

| | Intervention | Unit Dose | Dosage | | Route of | Regimen/ |
|---|---|---|---|---|---|---|
| Arm | Name | Strength(s) | Level(s) | | Administration | Treatment Period/ |
| Arm 1 | Pembrolizumab- | 231 mg | 650 | mg | SC | 3.94 mL SC Q6W Day 1 of |
| | HLN | deliverable dose | | | | Cycle 1 and Cycle 3 |
| | SC 165 | per 1.4 mL vial | | | | |
| | mg/mL | | | | | |
| Arm 2 | Pembrolizumab- | 182 mg | 650 | mg | SC | 5 mL SC Q6W Day 1 of |
| | HLN | deliverable dose | | | | Cycle 1 and Cycle 3 |
| | SC 130 | per 1.4 mL vial | | | | |
| | mg/mL | | | | | |
| Arms 1 | pembrolizumab | 100 mg/vial | 400 | mg | IV Infusion | 400 mg IV Q6W from |
| and 2 | | | | | | Cycles 2 and 4-18 |
| Arms 1 | pemetrexed | 100 mg vial | 500 | mg/m² | IV Infusion | 500 mg/m² IV Q3W |
| and 2 | | 500 mg vial | | | | |
| Arms 1 | carboplatin | 1 mL, 10 mg | AUC 5 | | IV Infusion | Day 1 of each 21-day cycle |
| and 2 | | | mg/mL/min | | | for 4 cycles |
| | | | nonsquamous | | | |
| | | | AUC 6 | | | |
| | | | mg/mL/min | | | |
| | | | squamous | | | |
| Arms 1 | paclitaxel | 1 mL, 6 mg | 200 | mg/m² | IV Infusion | Day 1 of each 21-day cycle |
| and 2 | | | | | | for 4 cycles |
| Arms 1 | nab- | 100 mg of | 100 | mg/m² | IV Infustion | Day 1, 8, 15 of each 21-day |
| and 2 | paclitaxel | paclitaxel in | | | | cycle for 4 cycles |
| | | single-use vial | | | | |
| | | for | | | | |
| | | reconstitution | | | | |
| Arms 1 | Arms 1 and | 5 mg tablet | 5 | mg | Oral | BID continuously |
| and 2 B | 2 B | | | | | |
| Arms 1 | cisplatin | 50 mg vial | 75 | mg/m² | IV Infusion | Day 1 of each 21-day cycle |

| | |
|---|---|
| and 2 B | for 4 cycles |

Serum concentration data from 81 subjects collected through cycles 1 (i.e., weeks 0-6) and 2 (i.e., weeks 7-12) from the Phase I clinical trial were used to characterize the PK of SC pembrolizumab given as the coformulation with hyaluronidase, along with extensive historical pembrolizumab IV PK data using population PK analysis. Non-linear mixed-effects modeling was applied to the Phase 1 data with priors from the previously established pembrolizumab reference PK model. The reference pembrolizumab PK model was based on pembrolizumab PK data collected from 2993 patients with various cancers who received pembrolizumab doses of 1 to 10 mg/kg Q2W, 2 to 10 mg/kg Q3W, or 200 mg Q3W in Phase I or Phase III clinical studies. The pembrolizumab absorption phase PK parameters were estimated for SC administration from the Phase I data, and any differences between the two solution strengths were also evaluated. Distribution and elimination parameters (Clearance (CL), central volume of distribution (Vc), inter-compartmental clearance (Q), and peripheral volume of distribution (Vp)) were fixed from the reference IV model, since these phases are the same for IV and SC administrations. Given the small sample size and short duration of the SC administrations in the study i.e., two treatment cycles, parameters describing the time-dependency and effects of patient baseline characteristics on pembrolizumab PK were also fixed from the previously established reference IV PK model.

The new population PK model developed was able to simultaneously describe pembrolizumab PK after IV or SC (with hyaluronidase) administrations. The final parameter estimates of the combined SC and IV population PK model are displayed in Table 8. The absorption phase for SC administration was characterized by a first order absorption rate (ka) and bioavailability (F) parameters. Distribution and elimination phases were described by a two-compartment model with time-dependent clearance and a fixed effect of body weight as established historically in the reference pembrolizumab PK model. Inclusion of a covariate effect for the strength of SC solution was not statistically significant, indicating no meaningful difference in the bioavailability and absorption rate between the two pembrolizumab-HLN SC solution strengths (i.e., Arm 1 165 mg/mL and Arm 2 130 mg/ml). Goodness of fit evaluation demonstrated the absence of structural bias as a function of drug concentration or time. The analysis showed that pembrolizumab-HLN when administered SC had an estimated bioavailability of 57% (range: 38% to 75%). Median time to achieve maximum pembrolizumab serum concentration in the subcutaneous formulation with hyaluronidase was estimated to be 4 days (range, 2 to 35 days). In addition, anti-drug antibodies (ADA) were observed in 1 of 83 (<2%) subjects in the Phase I study.

TABLE 8

| | | | |
|---|---|---|---|
| Parameter | Value | % RSE | % CV |

Estimated parameters using Phase 1 study data

| | | | |
|---|---|---|---|
| Ka (/day) [absorption rate] | 0.279 | 5.81 | 42.4 |
| F [bioavailability] | 0.572 | 3.04 | 20.9 |
| Residual error, SC | 0.274 | 2.47 | |
| Residual error, IV | 0.211 | 6.02 | |

Fixed parameters from reference IV model

| | | | |
|---|---|---|---|
| CL (L/day) | 0.281 | — | 30.6 |
| Vc (L) | 3.53 | — | 19.1 |
| Q (L/day) | 0.889 | — | 30.6 |
| Vp (L) | 2.75 | — | 19.1 |
| Maximum effect of time on CL | −0.218 | — | 79.5 |
| TI₅₀ (day) | 65.5 | — | |
| Hill | 2.99 | — | |
| α for CL and Q (allometric scaling factor) | 0.534 | — | |
| α for Vc and Vp (allometric scaling factor) | 0.514 | — | |
| Albumin effect on CL | −0.849 | — | |
| eGFR effect on CL | 0.123 | — | |
| Sex effect on CL | −0.162 | — | |
| Baseline ECOG effect on CL | −0.0697 | — | |
| Baseline tumor size effect on CL | 0.0933 | — | |
| Bilirubin effect on CL | −0.0488 | — | |
| Albumin effect on Vc | −0.233 | — | |
| Sex effect on Vc | −0.131 | — | |
| Tumor type effect (NSCLC vs other) on Vc | −0.059 | — | |

CV, coefficient of variation of between-subject distributions of parameters; eGFR, estimated glomerular filtration rate; NSCLC, non-small cell lung cancer; RSE, relative standard error; TI₅₀, time at which 50% of the maximum effect on clearance has been achieved. Presented population mean parameter estimates represent a typical patient with mean baseline characteristics.

The ongoing results of the Phase 1 study show that there are no new systemic safety signals for SC pembrolizumab-HLN. All injection site reactions were nonserious, mostly mild (Grade 1), and effectively managed. The overall safety profile during both SC pembrolizumab-HLN and pembrolizumab IV cycles were consistent with the known safety profiles of pembrolizumab monotherapy and the standard of care treatments administered in participants with NSCLC and RCC.

The two tested SC solution strengths of pembrolizumab-HLN (130 mg/ml and 165 mg/ml) had similar absorption PK. SC administration of pembrolizumab-HLN was well tolerated with no significant ADAs. Based upon an analysis of the PK model-based simulations using the estimated bioavailability and between-subject variability from this study it is believed that a subcutaneous dose of pembrolizumab-HLN of 760 to 790 mg Q6W should lead to similar exposure as the approved dose of 400 mg Q6W of pembrolizumab IV.

### Example 2

A six-weekly (Q6W) dosing schedule for SC pembrolizumab co-formulated with hyaluronidase across multiple tumor types based on an evaluation using modeling and simulation with 400 mg Q6W IV as a reference regimen.

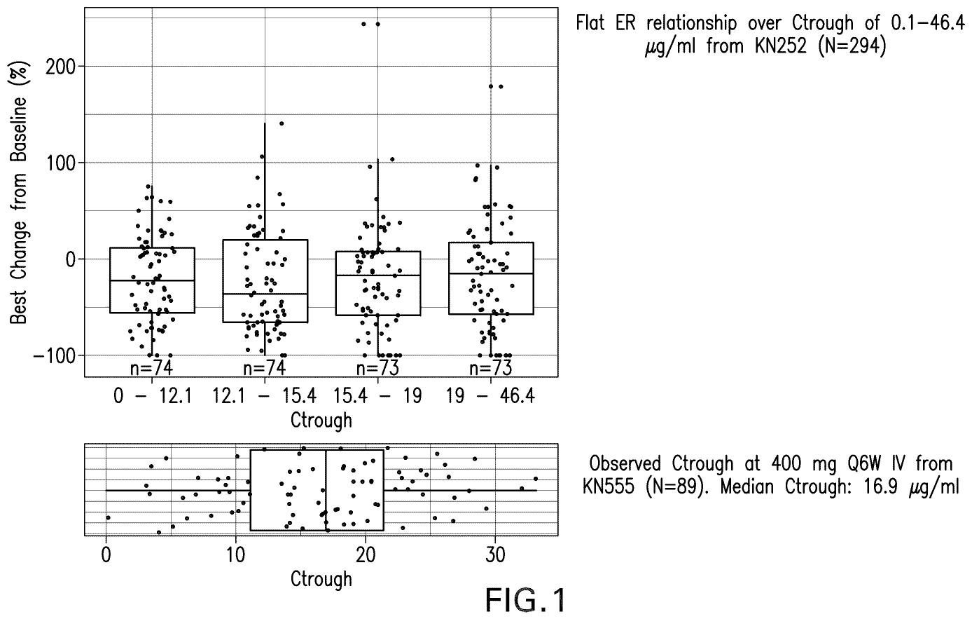

Pembrolizumab, an anti-PD-1 checkpoint inhibitor currently approved for use in multiple cancer indications, has demonstrated safety and efficacy when administered by intravenous infusion (IV) at a dose of 200 mg Q3W, 2 mg/kg Q3W or 400 mg Q6W. The robust characterization of pembrolizumab pharmacokinetics (PK) and exposure (concentration)-response (E-R) relationships for both efficacy and safety allow the use of model-based approaches to support alternative routes of administration for pembrolizumab. Previously, KN-252 efficacy data in 294 advanced melanoma patients showed flat exposure-response relationship over a Ctrough range of 3.5-32.5 ug/ml at the 200 mg Q3W IV dose of pembrolizumab. KN-555 PK data in 89 advanced melanoma patients shows observed Ctrough of 400 mg Q6W IV is well within the range of the previously established flat exposure-response relationship. Hence Ctrough of 400 mg Q6W IV is sufficient to maximize pembrolizumab efficacy [FIG. 1].

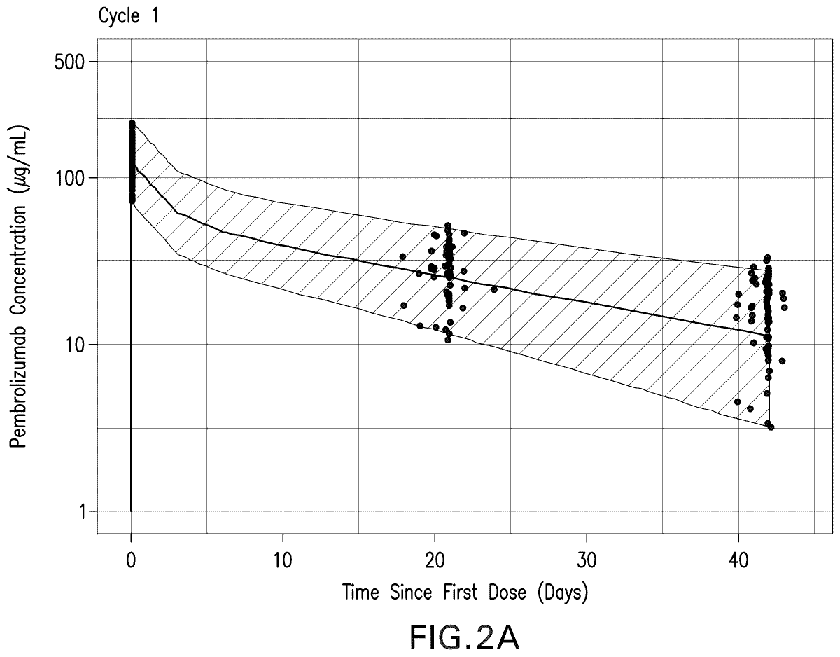

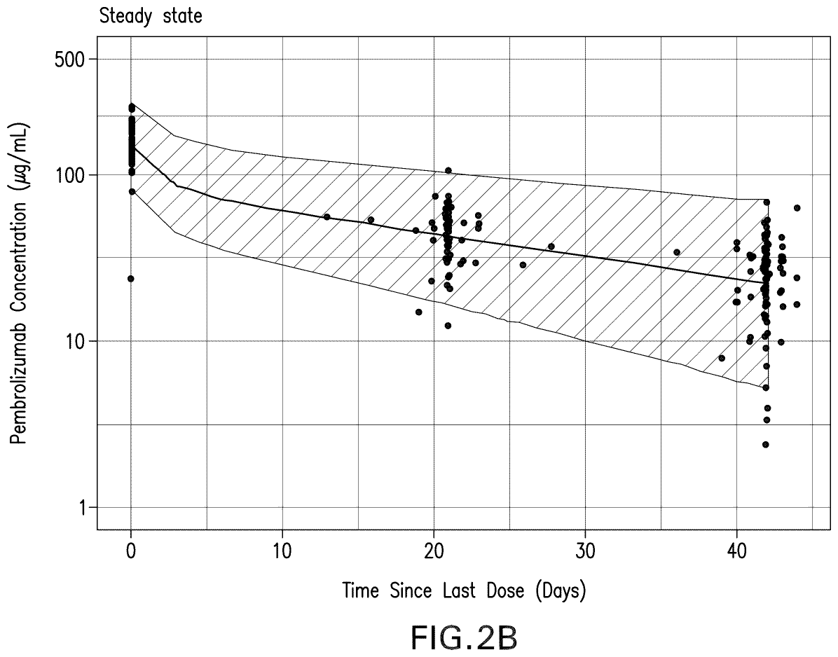

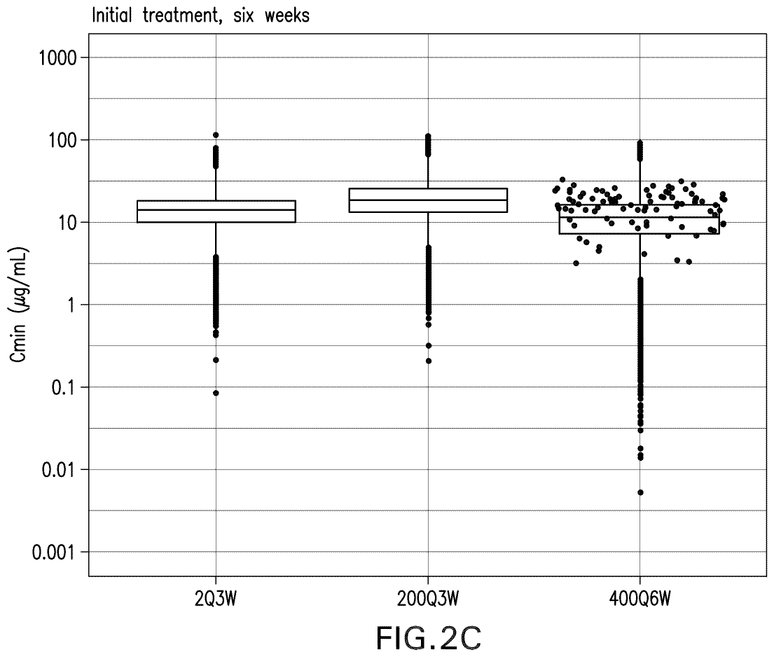

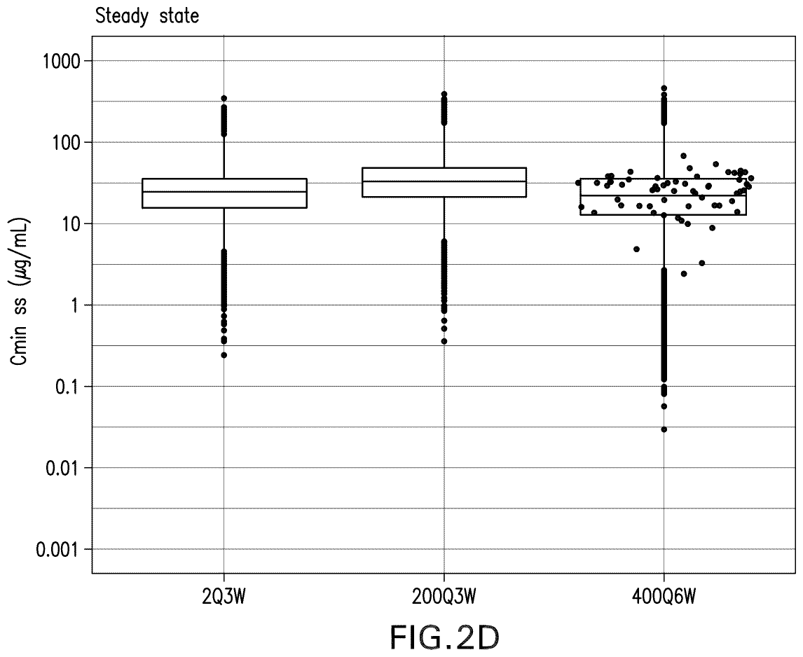

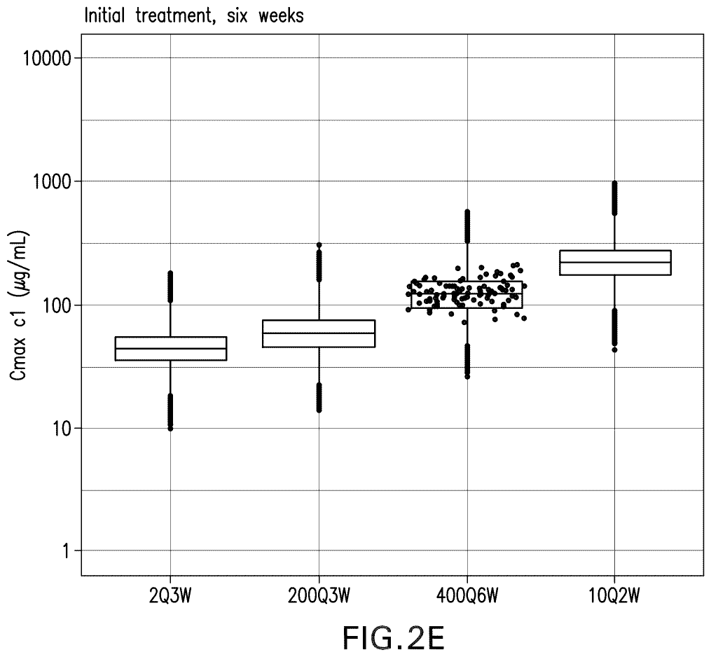

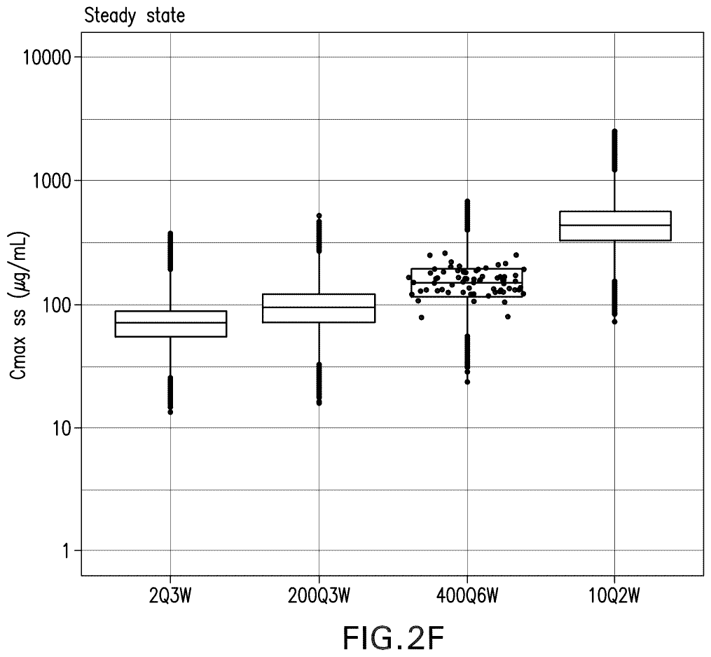

KEYNOTE-555 (NCT03665597) was an open-label, multicohort, phase 1 clinical trial investigating pembrolizumab in patients with advanced melanoma. Cohort B investigated the pharmacokinetics, efficacy, and safety of intravenous pembrolizumab at a dose of 400 mg every 6 weeks. 101 patients were screened and received treatment. A clinically meaningful objective response rate and durable progression-free survival within the expected range for first-line pembrolizumab was observed. Objective response rate was 50.5% (95% CI, 40.4-60.6); 19 patients (18.8%) had complete response and 32 (31.7%) had partial response. Median duration of response was not reached (NR; range, 2.4+ to 21.0+ months). Median progression-free survival was 13.8 months (95% CI, 4.1-NR). Observed pharmacokinetic exposures were consistent with model predictions for pembrolizumab 400 mg Q6W IV and within exposures at other approved and tested regimens (2 mg/kg or 200 mg Q3W IV and 10 mg/kg Q2W IV) (see FIG. 2). Safety in Cohort B was comparable to the known profile of pembrolizumab in melanoma with the 200 mg Q3W IV regimen. Grade 3-5 treatment-related adverse events occurred in 13 patients (12.9%). Three patients (3.0%) discontinued due to treatment-related adverse events.

The phase 2 KEYNOTE-B68 trial investigated the efficacy and safety of pembrolizumab administered 400 mg every six weeks IV in relapsed/refractory (R/R) classical Hodgkin lymphoma (cHL) or primary mediastinal B-cell lymphoma (PMBCL). At data cut-off, 66 pts (60 R/R cHL, 6 R/R PMBCL) were enrolled. Median follow-up (range) was 8.9 months (1-15.9) for pts with R/R cHL and 10.6 months (5.1-15.4) with R/R PMBCL. The ORR was 65% (95% CI, 51.6-76.9 [33.3% CR; 31.7% PR]) for pts with R/R cHL, and 50% (95% CI, 11.8-88.2 [33.3% CR; 16.7% PR]) for R/R PMBCL. Drug-related adverse events (AE) occurred in 24 pts (40%) with R/R cHL and 2 pts (33.3%) with R/R PMBCL. Grade ≥3 drug-related AEs occurred in 3 pts (5%) with R/R cHL and 1 pt (16.7%) with R/R PMBCL. Immune-mediated AEs occurred in 13 pts (21.7%) with R/R cHL and 1 pt (16.7%) with R/R PMBCL. Grade ≥3 immune-mediated AEs occurred in 2 pts (3.3%) with R/R cHL. With approximately 9 months of follow up, pembrolizumab 400 mg Q6W IV demonstrated robust antitumor activity in pts with R/R cHL and R/R PMBCL with no new safety concerns. The ORR observed was similar to pembrolizumab 200 mg Q3W IV in R/R cHL and R/R PMBCL.

PK model-based simulations were performed to select the SC dose, targeting consistency of the SC PK exposure profile with that of the approved 400 mg Q6W IV dose and overall exposure profiles based on clinical experience with pembrolizumab IV. The simulations were performed on a pooled dataset (total N=3105 subjects) of phase 1 studies evaluating pembrolizumab-HLN SC, and the reference pembrolizumab IV PK dataset including 2993 subjects with melanoma or non-small cell lung cancer (NSCLC) from previous Phase I and Phase III trials of IV pembrolizumab.

Pembrolizumab serum concentrations were simulated for doses ranging from 600 mg to 1200 mg Q6W of pembrolizumab-HLN SC and 400 mg Q6W of pembrolizumab IV from cycle 1 through cycle 3 (18 weeks, achieving steady state) using the combined SC and IV PK model (described in Table 8), including estimates of population mean PK parameters as well as between-subject-variability in each parameter and residual error. For each subject in the dataset, the simulated trough concentration at the end of the dosing interval (C_{trough}) and area under curve (AUC) exposure were determined both over Cycle 1 (first dose) and Cycle 3 (representing steady state). C_{trough} and AUC_{0-6 wks} indicate PK exposure measures and are regarded as drivers of pembrolizumab efficacy. Cycle 1 represents the PK exposures achieved after the first dose is administered. Cycle 3 represents the PK exposures achieved at steady state, which are the exposures that will then be maintained throughout treatment duration. The geometric mean (GM) of C_{trough} and AUC_{0-6 wks} was calculated for each SC dose and the 400 mg IV dose of pembrolizumab. Then, the geometric mean ratio (GMR) of SC versus IV pembrolizumab (as the ratio of GM of each formulation group) for both of these PK exposure measures were calculated, for treatment cycles 1 and 3.

PK model-based simulations indicate that pembrolizumab-HLN SC (with hyaluronidase PH20 variant 2) doses ranging from 600 to 1000 mg Q6W lead to comparable exposures as the approved dose of 400 mg Q6W of pembrolizumab administered via IV infusion, and are safe in view of the highest clinically evaluated dose at 10 mg/kg Q2W IV with established safety. In principle, similar PK exposures lead to similar efficacy and safety of pembrolizumab, given that the exposure-response relationships for both efficacy and safety are already well established for pembrolizumab. Hence, this range of SC doses is expected to have comparable efficacy and safety as 400 mg Q6W IV.

For the lower end of exposures (AUC or C_{trough}), establishing non-inferiority is adequate, using a generally accepted margin of a lower bound of 90% CI around GMR >0.8. This would ascertain that the efficacy of a dose of pembrolizumab administered subcutaneously co-formulated with hyaluronidase is no worse than the efficacy of the dose of pembrolizumab (400 mg Q6W) administered by an IV route of administration (“IV pembrolizumab”). The simulations developed showed that SC pembrolizumab-HLN doses of 600 mg Q6W or higher all had a mean SC:IV C_{trough} ratio greater than 1 and SC pembrolizumab-HLN doses of 600 mg Q6W or higher all had a mean SC:IV AUC ratio greater than 0.8. See Table 9A and 9B. Table 9A-B shows the SC (subcutaneous):IV (Intravenous) Geometric Mean (GM) Ratio (GMR) of C_{trough} in Cycle 1 and C_{trough} at steady state (Ctroughss) across different evaluated Q6W SC doses relative to 400 mg Q6W IV of pembrolizumab. Table 9C-D shows the SC:IV GMR of AUC_{0-6 wks} in Cycle 1 (AUC.C1) and at steady state (AUC.ss).

TABLE 9A
GM and GMR by Trial for C_{trough} Cycle 1

| Regimen | Geomean | Regimen.IV | Geomean.IV | GMR |
|---|---|---|---|---|
| 600 mg SC Q6W | 10.5 | 400 mg IV Q6W | 10.5 | 1.00 |
| 620 mg SC Q6W | 10.9 | 400 mg IV Q6W | 10.5 | 1.04 |
| 640 mg SC Q6W | 11.3 | 400 mg IV Q6W | 10.5 | 1.08 |
| 650 mg SC Q6W | 11.4 | 400 mg IV Q6W | 10.5 | 1.09 |
| 660 mg SC Q6W | 11.6 | 400 mg IV Q6W | 10.5 | 1.10 |
| 680 mg SC Q6W | 12.0 | 400 mg IV Q6W | 10.5 | 1.14 |
| 700 mg SC Q6W | 12.3 | 400 mg IV Q6W | 10.5 | 1.17 |
| 720 mg SC Q6W | 12.7 | 400 mg IV Q6W | 10.5 | 1.21 |
| 740 mg SC Q6W | 13.0 | 400 mg IV Q6W | 10.5 | 1.24 |
| 760 mg SC Q6W | 13.4 | 400 mg IV Q6W | 10.5 | 1.28 |
| 790 mg SC Q6W | 13.9 | 400 mg IV Q6W | 10.5 | 1.32 |
| 800 mg SC Q6W | 14.1 | 400 mg IV Q6W | 10.5 | 1.34 |
| 900 mg SC Q6W | 15.8 | 400 mg IV Q6W | 10.5 | 1.50 |
| 1000 mg SC Q6W | 17.6 | 400 mg IV Q6W | 10.5 | 1.68 |
| 1100 mg SC Q6W | 19.3 | 400 mg IV Q6W | 10.5 | 1.84 |
| 1200 mg SC Q6W | 21.1 | 400 mg IV Q6W | 10.5 | 2.01 |

TABLE 9B
GM and GMR by trial for C_{troughss}

| Regimen | GEOMEAN | Regimen IV | GEOMEAN.IV | GMR |
|---|---|---|---|---|
| 600 mg SC Q 6W | 17.4 | 400 mg IV Q6W | 17.7 | 0.983 |
| 620 mg SC Q 6W | 18.0 | 400 mg IV Q6W | 17.7 | 1.020 |
| 640 mg SC Q 6W | 18.6 | 400 mg IV Q6W | 17.7 | 1.050 |
| 650 mg SC Q 6W | 18.9 | 400 mg IV Q6W | 17.7 | 1.070 |
| 660 mg SC Q 6W | 19.2 | 400 mg IV Q6W | 17.7 | 1.080 |
| 680 mg SC Q 6W | 19.7 | 400 mg IV Q6W | 17.7 | 1.110 |
| 700 mg SC Q 6W | 20.3 | 400 mg IV Q6W | 17.7 | 1.150 |
| 720 mg SC Q 6W | 20.9 | 400 mg IV Q6W | 17.7 | 1.180 |
| 740 mg SC Q 6W | 21.5 | 400 mg IV Q6W | 17.7 | 1.210 |
| 760 mg SC Q 6W | 22.1 | 400 mg IV Q6W | 17.7 | 1.250 |
| 790 mg SC Q 6W | 22.9 | 400 mg IV Q6W | 17.7 | 1.290 |
| 800 mg SC Q 6W | 23.2 | 400 mg IV Q6W | 17.7 | 1.310 |
| 900 mg SC Q 6W | 26.1 | 400 mg IV Q6W | 17.7 | 1.470 |
| 1000 mg SC Q 6W | 29.0 | 400 mg IV Q6W | 17.7 | 1.640 |
| 1100 mg SC Q 6W | 31.9 | 400 mg IV Q6W | 17.7 | 1.800 |
| 1200 mg SC Q 6W | 34.8 | 400 mg IV Q6W | 17.7 | 1.970 |

TABLE 9C
GM and GMR by Trial for AUC Cycle 1

| Regimen | Geomean | Regimen.IV | Geomean.IV | GMR |
|---|---|---|---|---|
| 600 mg SC Q6W | 1060 | 400 mg IV Q6W | 1310 | 0.809 |
| 620 mg SC Q6W | 1100 | 400 mg IV Q6W | 1310 | 0.840 |
| 640 mg SC Q6W | 1130 | 400 mg IV Q6W | 1310 | 0.863 |
| 650 mg SC Q6W | 1150 | 400 mg IV Q6W | 1310 | 0.878 |
| 660 mg SC Q6W | 1170 | 400 mg IV Q6W | 1310 | 0.893 |
| 680 mg SC Q6W | 1200 | 400 mg IV Q6W | 1310 | 0.916 |
| 700 mg SC Q6W | 1240 | 400 mg IV Q6W | 1310 | 0.947 |
| 720 mg SC Q6W | 1270 | 400 mg IV Q6W | 1310 | 0.969 |
| 740 mg SC Q6W | 1310 | 400 mg IV Q6W | 1310 | 1.000 |
| 760 mg SC Q6W | 1340 | 400 mg IV Q6W | 1310 | 1.020 |
| 790 mg SC Q6W | 1400 | 400 mg IV Q6W | 1310 | 1.070 |
| 800 mg SC Q6W | 1410 | 400 mg IV Q6W | 1310 | 1.080 |
| 900 mg SC Q6W | 1590 | 400 mg IV Q6W | 1310 | 1.210 |
| 1000 mg SC Q6W | 1770 | 400 mg IV Q6W | 1310 | 1.350 |
| 1100 mg SC Q6W | 1940 | 400 mg IV Q6W | 1310 | 1.480 |
| 1200 mg SC Q6W | 2120 | 400 mg IV Q6W | 1310 | 1.620 |

TABLE 9D
GM and GMR by trial for AUCss

| Regimen | Geomean | Regimen IV | Geomean IV | GMR |
|---|---|---|---|---|
| 600 mg SC Q6W | 1550 | 400 mg IV Q6W | 1840 | 0.842 |
| 620 mg SC Q6W | 1600 | 400 mg IV Q6W | 1840 | 0.870 |
| 640 mg SC Q6W | 1650 | 400 mg IV Q6W | 1840 | 0.897 |
| 650 mg SC Q6W | 1680 | 400 mg IV Q6W | 1840 | 0.913 |
| 660 mg SC Q6W | 1710 | 400 mg IV Q6W | 1840 | 0.929 |
| 680 mg SC Q6W | 1760 | 400 mg IV Q6W | 1840 | 0.957 |
| 700 mg SC Q6W | 1810 | 400 mg IV Q6W | 1840 | 0.984 |
| 720 mg SC Q6W | 1860 | 400 mg IV Q6W | 1840 | 1.010 |
| 740 mg SC Q6W | 1910 | 400 mg IV Q6W | 1840 | 1.040 |
| 760 mg SC Q6W | 1970 | 400 mg IV Q6W | 1840 | 1.070 |
| 790 mg SC Q6W | 2040 | 400 mg IV Q6W | 1840 | 1.110 |
| 800 mg SC Q6W | 2070 | 400 mg IV Q6W | 1840 | 1.120 |
| 900 mg SC Q6W | 2330 | 400 mg IV Q6W | 1840 | 1.270 |
| 1000 mg SC Q6W | 2590 | 400 mg IV Q6W | 1840 | 1.410 |
| 1100 mg SC Q6W | 2840 | 400 mg IV Q6W | 1840 | 1.540 |
| 1200 mg SC Q6W | 3100 | 400 mg IV Q6W | 1840 | 1.680 |

SC pembrolizumab-HLN doses ranging from 760 to 790 mg Q6W have similar PK exposures as the approved dose of 400 mg Q6W of pembrolizumab administered via IV infusion. Based upon the analyses conducted, efficacy of SC pembrolizumab-HLN at the dose of 760 to 790 mg Q6W is expected to be similar to that of 400 mg Q6W IV pembrolizumab based on the following:

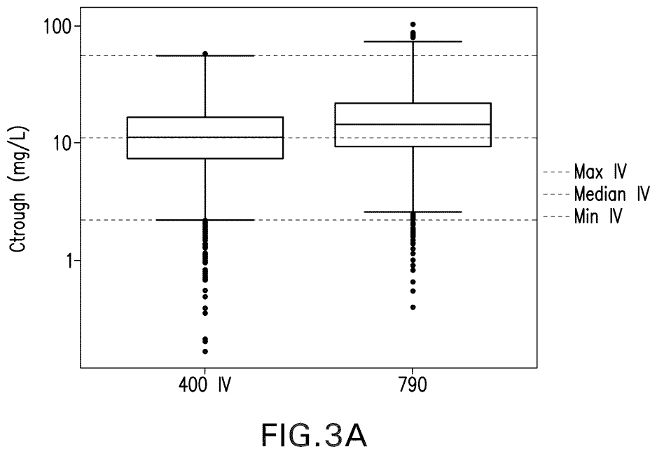

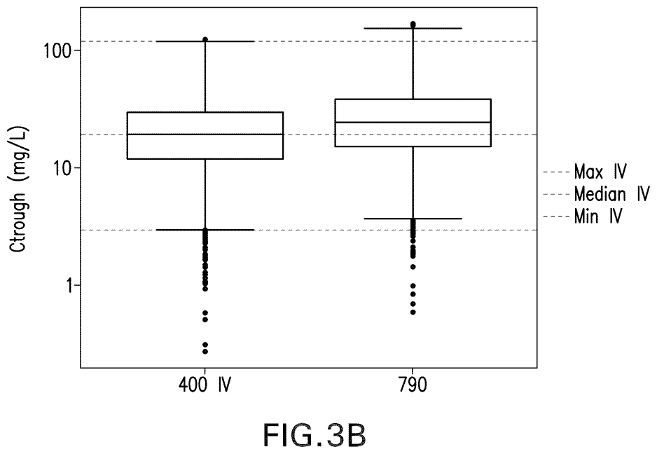

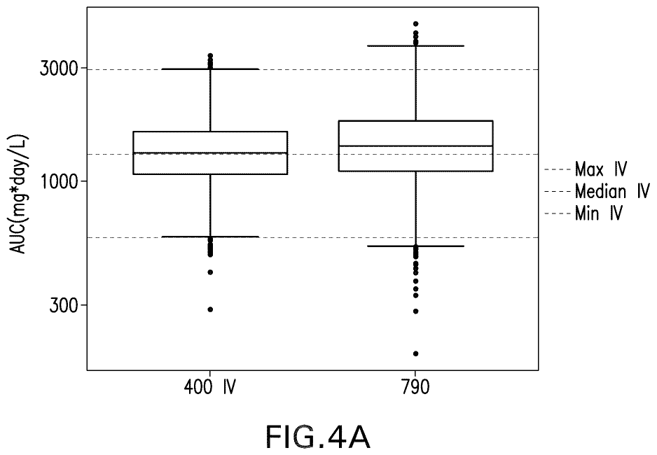

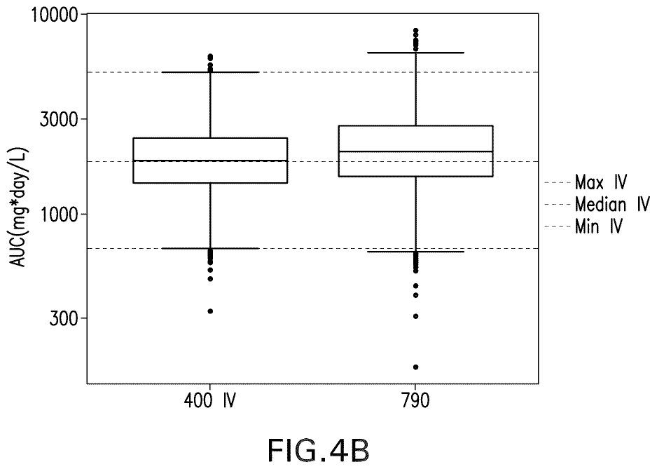

C_{trough} at a 760 to 790 mg Q6W SC dose is expected to be ~25 to 30% higher than 400 mg Q6W IV, through treatment duration. Moreover, the distributions of C_{trough} largely overlap between SC and IV at both Cycle 1 and steady state. See FIGS. 3A and 3B. AUC_{0-6 wks} exposure at a 760 to 790 mg Q6W SC dose is expected to be ~2 to 10% higher than 400 mg Q6W IV, through treatment duration. Moreover, the distributions of AUC_{0-6 wks} largely overlap between SC and IV at both Cycle 1 and steady state. See FIGS. 4A and 4B.

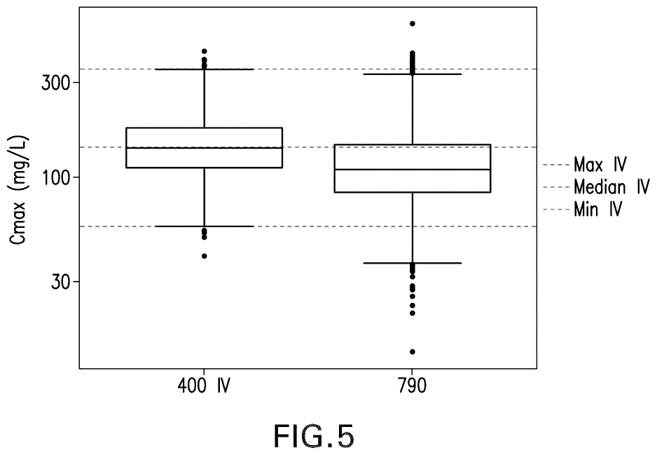

In the case of comparing SC pembrolizumab to IV pembrolizumab, it is important to note the key differences that inherently exist in the PK profiles between SC and IV administrations. Typically, for comparable doses (adjusted for bioavailability) the concentrations with SC administration gradually accrue over ~4 days, and the peak concentrations (Cₘₐₓ) after a SC administration of pembrolizumab are much lower than the Cₘₐₓ achieved at the end of an IV infusion. Specifically, with the 760 to 790 mg Q6W SC dose, the Cₘₐₓ is expected to be much reduced (~38 to 35% lower at Cycle 1 and ~25 to 22% lower at steady state) than the Cₘₐₓ achieved at 400 mg Q6W IV. See FIG. 5. Thus, with the pembrolizumab-HLN SC dose range, there is no increase expected in Cₘₐₓ throughout treatment relative to the approved IV dose of 400 mg Q6W. Further, all PK exposures of 760 to 790 mg Q3W SC pembrolizumab-HLN will certainly remain well below the 5-fold higher dose/exposures at 10 mg/kg Q2W IV, which is the highest clinically evaluated dose with established safety. Therefore, the safety profile after SC administration of 760 to 790 mg Q6W SC pembrolizumab-HLN is unlikely to differ from the safety profile previously established for the 400 mg Q6W IV pembrolizumab dose identified, and hence quantitative assessment for the upper bound of exposures was not further evaluated.

The selected dose for phase III clinical trials of pembrolizumab SC with hyaluronidase PH20 variant 2 is 790 mg Q6W. FIGS. 3A and 3B summarize the results of the population simulation including variability for C_{trough} for a dose of 790 mg Q6W SC and 400 mg Q6W IV of pembrolizumab. The simulations developed showed that the 790 mg Q6W SC dose leads to a range of C_{trough} across different patients that generally overlaps with the 400 mg Q6W IV dose. FIGS. 4A and 4B summarize the results of the population simulation including variability for AUC_{0-6 wks} for a dose of 790 mg Q6W SC and 400 mg Q6W IV of pembrolizumab. The simulations showed that the 790 mg Q6W SC dose leads to a range of AUC_{0-6 wks} across different patients that generally overlaps with the 400 mg Q6W IV dose. FIG. 5 summarizes the results of the population simulation including variability for Cₘₐₓ for a dose of 790 mg Q6W SC and 400 mg Q6W IV of pembrolizumab. The simulations showed that the 790 mg Q6W SC dose leads to a range of Cₘₐₓ across different patients that is generally lower than the 400 mg Q6W IV dose. FIGS. 3A and 3B, 4A and 4B depict the distribution (5^{th}, 25^{th}, 50^{th}, 75^{th}, and 95^{th} percentiles) at cycle 1 and steady state of C_{trough} and AUC_{0-6 wks} respectively, and FIG. 5 depicts the distribution (5^{th}, 25^{th}, 50^{th}, 75^{th}, and 95^{th} percentiles) at steady state of Cₘₐₓ using PK model-based simulations at a dose of 790 mg Q6W SC and 400 mg Q6W IV of pembrolizumab. Simulated PK exposure measures in 3105 subjects are shown.

Overall, the model-based simulations as supported by Tables 9A-D, FIGS. 3A, 3B, 4A, 4B and 5 indicate that a dose of 790 mg Q6W of pembrolizumab-HLN administered SC should lead to an optimal PK exposure profile that is similar to that of the approved dose of 400 mg Q6W of pembrolizumab IV, thus maintaining efficacy, while also remaining within the clinical safety margin.

### Example 3

A Phase 3 Randomized, Open-label Clinical Study to Evaluate the Pharmacokinetics and Safety of Subcutaneous Pembrolizumab Co-formulated With Hyaluronidase (pembrolizumab-HLN) Versus Intravenous Pembrolizumab, Administered With Chemotherapy, in the First-line Treatment of Participants With Metastatic Non-small Cell Lung Cancer

This is a Phase 3, randomized, active-controlled, parallel-group, multisite, open-label study of pembrolizumab-HLN and platinum doublet chemotherapy vs pembrolizumab IV and platinum doublet chemotherapy in participants with treatment-naïve metastatic NSCLC.

After a screening period of up to 28 days, participants will be randomly assigned in a 2:1 ratio to Arm 1 or Arm 2. One cycle is 6 weeks. Arm 1 is pembrolizumab-HLN Q6W for up to 18 cycles in combination with platinum doublet chemotherapy. Arm 2 is Pembrolizumab IV Q6W for up to 18 cycles in combination with platinum doublet chemotherapy.

Platinum doublet chemotherapy is as follows: For Nonsquamous NSCLC: Up to 4 infusions of pemetrexed Q3W with a platinum chemotherapy (cisplatin Q3W or carboplatin Q3W), followed by pemetrexed maintenance until one of the conditions for discontinuation of study intervention is met. For Squamous NSCLC: Up to 4 infusions of carboplatin Q3W with a taxane (paclitaxel Q3W or nab-paclitaxel [Days 1, 8, 15, 22, 29, and 36 of Cycles 1 and 2]).

Randomization will be stratified by ECOG performance status, histology, PD-L1 TPS, and region. Each participant will receive study intervention until one of the conditions for discontinuation of study intervention is met.

The dual primary objectives of the study are to compare Cycle 1 AUC_{0-6 wks} and steady-state (Cycle 3) C_{trough} for pembrolizumab-HLN vs pembrolizumab IV at Q6W dosing regimens. Noninferiority will be evaluated with the noninferiority margin of 0.8. Tumor response will be evaluated per an adjustment to RECIST 1.1. Safety assessments will also be performed.

Pembrolizumab IV in combination with platinum doublet chemotherapy is indicated for the first-line treatment of patients with metastatic NSCLC. This study will primarily compare pembrolizumab exposures between pembrolizumab-HLN (Arm 1) and pembrolizumab IV (Arm 2) over Q6W dosing regimens, both in combination with chemotherapy, in participants with treatment-naïve metastatic NSCLC. Additional descriptive comparisons of pembrolizumab C_{trough} between pembrolizumab-HLN and pembrolizumab 200 mg IV Q3W will also be made using model-based exposures.
Study Objectives, and Endpoints:
In participants with treatment-naïve metastatic NSCLC:

TABLE 10

| Objectives | Endpoints |
|---|---|
| Primary | |
| Objective: To compare pembrolizumab-HLN to | Cycle 1 AUC_{0-6 wks} |
| pembrolizumab IV with respect to Cycle 1 AUC | |
| Expected outcome: pembrolizumab-HLN is | |
| noninferior to pembrolizumab IV with respect to | |
| geometric mean pembrolizumab Cycle 1 AUC | |
| Objective: To compare pembrolizumab-HLN to | Steady-state (Cycle 3) C_{trough} |
| pembrolizumab IV with respect to steady-state | The primary analysis will be performed on the |
| (Cycle 3) C_{trough} | model-based values of C_{trough} |
| Expected outcome: pembrolizumab-HLN is | |
| noninferior to pembrolizumab IV with respect to | |
| geometric mean pembrolizumab steady-state | |
| (Cycle 3) C_{trough} | |
| Secondary | |
| To evaluate pembrolizumab exposure for | Cycle 1: Cₘₐₓ and C_{trough} |
| pembrolizumab-HLN relative to pembrolizumab | Steady state (Cycle 3): AUC_{0-6 wks} and Cₘₐₓ |
| IV Q6W | |
| To evaluate the development of circulating anti- | Anti-pembrolizumab antibodies |
| pembrolizumab antibodies for pembrolizumab- | |
| HLN and pembrolizumab IV | |
| To evaluate pembrolizumab C_{trough} for | Cycle 1: Model-based C_{trough} |
| pembrolizumab-HLN relative to pembrolizumab | Steady state: Model-based C_{trough} |
| IV Q3W | |
| To evaluate pembrolizumab-HLN and | Objective response: CR or PR |
| pembrolizumab IV with respect to ORR per | |
| RECIST 1.1 as assessed by BICR | |
| To evaluate pembrolizumab-HLN and | PFS: The time from randomization to the first |
| pembrolizumab IV with respect PFS per | documented disease progression or death due to |
| RECIST 1.1 as assessed by BICR | any cause, whichever occurs first |
| To evaluate pembrolizumab-HLN and | OS: The time from randomization to death due |
| pembrolizumab IV with respect to OS | to any cause |
| To evaluate pembrolizumab-HLN and | DOR: The time from the first documented |
| pembrolizumab IV with respect DOR per | evidence of CR or PR until disease progression |
| RECIST 1.1 as assessed by BICR | or death due to any cause, whichever occurs |
| | first |
| To evaluate the safety and tolerability of | AE |
| pembrolizumab-HLN and pembrolizumab IV | Discontinuation of study intervention due to |
| | AEs |

TABLE 11
Overall Study Design

| Intervention Group | | Dose | | Dose | Route of | Treatment |
|---|---|---|---|---|---|---|
| Name | Drug | Strength | | Frequency | Administration | Period |
| Arm 1 | Pembrolizumab- | 790 | mg | Q6W | SC | 18 cycles |
| (nonsquamous and | HLN | | | | | |
| squamous) | | | | | | |
| Arm 2 | Pembrolizumab | 400 | mg | Q6W | IV | 18 cycles |
| (nonsquamous and | | | | | | |
| squamous) | | | | | | |
| Arms 1&2 | Pemetrexed | 500 | mg/m² | Q3W | IV | No limit |
| (nonsquamous) | | | | | | |
| Arms 1&2 | Cisplatin | 75 | mg/m² | Q3W | IV | 2 cycles |
| (nonsquamous) | | | | | | |
| Arms 1&2 | Carboplatin | AUC5-6^{b} | | Q3W | IV | 2 cycles |
| (nonsquamous and | | | | | | |
| squamous) | | | | | | |
| Arms 1&2 | Paclitaxel | 200 | mg/m² | Q3W | IV | 2 cycles |
| (squamous) | | | | | | |
| Arms 1&2 | Nab-paclitaxel | 100 | mg/m² | Days 1, 8, 15, | IV | 2 cycles |

| | |
|---|---|
| (squamous) | 22, 29, and 36 |
| | of Cycles 1 |
| | and 2 |

AUC = area under the curve;
IV = intravenous;
Q3W = every 3 weeks;
Q6W = every 6 weeks;
SC = subcutaneous.
Cycle = 6 weeks
^{b}AUC 5 mg/ml/min for nonsquamous; AUC 6 mg/ml/min for squamous

### Rationale for Pharmacokinetic Endpoints

The dual primary endpoints of Cycle 1 AUC_{0-6 wks} and steady state (Cycle 3) C_{trough} will be used to compare pembrolizumab exposures between pembrolizumab-HLN and pembrolizumab IV Q6W. Cycle 1 AUC_{0-6 wks} is the most conservative PK value to ensure noninferiority of SC exposure relative to IV. Any differences in exposure between SC and IV administrations of pembrolizumab should reduce after multiple dosing due to accumulation. Therefore, demonstrating noninferiority of SC AUC exposure at Cycle 1 will imply noninferiority at steady-state as well. Based upon our analyses, the higher AUC exposure at steady-state following SC dosing is not expected to exceed the established clinical safety margin for pembrolizumab (i.e., exposure at 10 mg/kg Q2W IV).

Steady-state (Cycle 3) C_{trough} is the concentration at the end of the 6-week dosing interval after the third dose, which is steady state for pembrolizumab. Because the pharmacological activity of mAbs is mediated through direct interaction with a specific target, target saturation can be used as a surrogate for maximal pharmacologic and therapeutic activity. Pembrolizumab exposures at the approved IV doses are expected to maintain PD-1 target saturation throughout the dosing interval and thereby efficacy. Therefore, C_{trough} for the approved IV dose of 400 mg Q6W can be considered a threshold above which target saturation and efficacy will be maintained. Demonstration of noninferiority of pembrolizumab steady-state (Cycle 3) C_{trough} for pembrolizumab-HLN would enable the inference that efficacy similar to pembrolizumab IV dosing will be maintained.

The secondary PK endpoints will enable further characterization of pembrolizumab exposure: For comparison with pembrolizumab IV Q6W: Cycle 1: Cₘₐₓ, C_{trough}; Steady state (Cycle 3): AUC_{0-6 wks} and Cₘₐₓ. For comparison with pembrolizumab IV Q3W: Model-based C_{trough} at Cycle 1 and steady state.

PK model-based simulations indicate that a pembrolizumab SC dose of 790 mg Q6W leads to similar exposures as the approved pembrolizumab IV dose of 400 mg Q6W. Efficacy is expected to be retained with SC pembrolizumab at the dose of 790 mg Q6W based on the following:
C_{trough} at a 790 mg Q6W SC dose is expected to be approximately 30% higher than 400 mg Q6W IV throughout the treatment duration. Moreover, the distributions of C_{trough} overlap between SC and IV at both Cycle 1 and steady state (See FIGS. 3A and 3B).
AUC_{0-6 wks} exposure at a 790 mg Q6W SC dose is expected to be approximately 10% higher than 400 mg Q6W IV throughout the treatment duration. Moreover, the distributions of AUC_{0-6 wks} overlap between SC and IV at both Cycle 1 and steady state (See FIGS. 4A and 4B).

Safety is expected to be maintained with SC pembrolizumab at the dose of 790 mg Q6W based on the following:
Cₘₐₓ is expected to be lower (by approximately 35% at Cycle 1 and approximately 22% at steady state) than the Cₘₐₓ achieved at 400 mg Q6W IV (FIG. 5). Therefore, there is no increase in Cₘₐₓ expected throughout the duration of treatment relative to the approved dose of 400 mg Q3W IV.
All SC exposures (Cₘₐₓ, C_{avg}, C_{trough}) over the dosing interval of 6 weeks and throughout the duration of treatment are expected to remain below the Cₘₐₓ and initial concentrations of 400 mg Q6W IV and well below the highest dose and exposures established for clinical safety (i.e., 10 mg/kg Q2W).

Overall, as discussed above, the model-based simulations developed indicate that a dose of 790 mg Q6W of pembrolizumab-HLN administered subcutaneously should lead to an optimal PK exposure profile that is similar to that of the approved dose of 400 mg Q6W of pembrolizumab IV, thus maintaining efficacy, while also remaining within the clinical safety margin.

### Example 4

A three-weekly (Q3W) dosing schedule for SC pembrolizumab-HLN across multiple tumor types based on an evaluation using modeling and simulation with Q6W SC pembrolizumab-HLN as a reference regimen

PK model-based simulations developed indicate that a dosing regimen of SC pembrolizumab with the hyaluronidase PH20 variant 2 of 790 mg Q6W leads to exposures similar to the approved dose of 400 mg Q6W of pembrolizumab administered via IV infusion. In principle, similar PK exposures lead to similar efficacy and safety of pembrolizumab, given that the exposure-response relationships for both efficacy and safety are already well established for pembrolizumab. Using such an exposure-matching approach, a Q3W dose of SC pembrolizumab-HLN was selected using PK model-based simulations, with the Q6W SC pembrolizumab-HLN dose as a reference.

PK model-based simulations were performed to select the Q3W SC dose, targeting consistency of the SC PK exposure profile with that of Q6W SC dose and overall exposure profiles based on clinical experience with pembrolizumab IV. The simulations were performed on a pooled dataset (total N=3105 subjects) of phase 1 studies evaluating pembrolizumab-HLN SC, and the reference pembrolizumab IV PK dataset including 2993 subjects with melanoma or non-small cell lung cancer (NSCLC) from previous Phase I and Phase III trials of IV pembrolizumab.

Pembrolizumab serum concentrations were simulated for doses ranging from 380 mg to 410 mg Q3W of pembrolizumab-HLN SC and a range of Q6W SC doses corresponding to double the Q3W doses from cycle 1 through cycle 6 or cycle 3 (18 weeks, achieving steady state) using the combined SC and IV PK model (described in Table 8), including estimates of population mean PK parameters as well as between-subject-variability in each parameter and residual error. For each subject in the dataset, the simulated trough concentration at the end of the dosing interval (C_{trough}) and area under curve (AUC) exposure were determined both over Cycle 1 (first dose) and steady state (Cycle 6 for Q3W or Cycle 3 for Q6W). C_{trough} and AUC indicate PK exposure measures and are regarded as drivers of pembrolizumab efficacy. Cycle 1 represents the PK exposures achieved after the first dose is administered. PK exposures achieved at steady state are the exposures that will then be maintained throughout treatment duration. The geometric mean (GM) of C_{trough} and AUC was calculated for each SC dose of pembrolizumab. Then, the geometric mean ratio (GMR) of SC versus IV pembrolizumab (as the ratio of GM of each formulation group) for both of these PK exposure measures were calculated, for treatment cycles 1 and steady state.

The simulations developed showed that Q3W SC pembrolizumab-HLN doses that are half of the corresponding Q6W SC pembrolizumab-HLN dose had Ctrough exceeding the Q6W SC dose from cycle 1 through steady state (mean SC-Q3W:SC-Q6W C_{trough} ratio greater than 1), comparable AUC exposure at cycle 1 (mean SC-Q3W:SC-Q6W AUC ratio greater than 0.8) and the same AUC exposure at steady state (mean SC-Q3W:SC-Q6W AUC ratio of 1). See Tables 12A-D. Tables 12A-B shows the Q3W:Q6W Geometric Mean Ratio (GMR) of C_{trough} in Cycle 1 and C_{trough} at steady state (C_{troughss}) across different evaluated Q3W and Q6W SC doses of pembrolizumab-HLN. In Table 12A, the sampling period of the SC-Q3W regimen was 3 weeks, and the sampling period for the SC-Q6W reference was 6 weeks. In Table 12B, the sampling period of the SC-Q3W regimen was 6 weeks, and the sampling period for the SC-Q6W reference was 6 weeks. Tables 12C-D shows the Q3W:Q6W GMR of AUC_{0-6 wks} in Cycle 1 (AUC.C1), Cycle 2, and at steady state (AUC.ss) across different evaluated Q3W and Q6W SC doses of pembrolizumab-HLN. In Tables 12C-D, the sampling period of the SC-Q3W regimen was 6 weeks, and the sampling period for the SC-Q6W reference was 6 weeks.

TABLE 12A
GM and GMR by trial for Ctrough Cycle 1 3W

| Regimen | GEOMEAN | Regimen REF | GEOMEAN.REF | GMR |
|---|---|---|---|---|
| 380 mg SC Q3W | 15.3 | 760 mg SC Q6W | 13.4 | 1.14 |
| 390 mg SC Q3W | 15.8 | 780 mg SC Q6W | 13.7 | 1.15 |
| 395 mg SC Q3W | 16.0 | 790 mg SC Q6W | 13.9 | 1.15 |
| 410 mg SC Q3W | 16.6 | 820 mg SC Q6W | 14.4 | 1.15 |

TABLE 12B
GM and GMR by trial for Ctrough ss

| Regimen | GEOMEAN | Regimen.REF | GEOMEAN.REF | GMR |
|---|---|---|---|---|
| 380 mg SC Q3W | 34.5 | 760 mg SC Q6W | 22.1 | 1.56 |
| 390 mg SC Q3W | 35.4 | 780 mg SC Q6W | 22.6 | 1.57 |
| 395 mg SC Q3W | 35.9 | 790 mg SC Q6W | 22.9 | 1.57 |
| 410 mg SC Q3W | 37.2 | 820 mg SC Q6W | 23.8 | 1.56 |

TABLE 12C
GM and GMR by trial for AUC.6w ss

| Regimen | GEOMEAN | Regimen.REF | GEOMEAN.REF | GMR |
|---|---|---|---|---|
| 380 mg SC Q3W | 1950 | 760 mg SC Q6W | 1970 | 0.990 |
| 390 mg SC Q3W | 2000 | 780 mg SC Q6W | 2020 | 0.990 |
| 395 mg SC Q3W | 2030 | 790 mg SC Q6W | 2040 | 0.995 |
| 410 mg SC Q3W | 2110 | 820 mg SC Q6W | 2120 | 0.995 |

TABLE 12D
GM and GMR by trial for AUC.6w Cycle 1/2

| Regimen | GEOMEAN | Regimen.REF | GEOMEAN.REF | Cycle | GMR |
|---|---|---|---|---|---|
| 380 mg SC Q3W | 1120 | 760 mg SC Q6W | 1340 | C1 | 0.836 |
| 380 mg SC Q3W | 1680 | 760 mg SC Q6W | 1730 | C2 | 0.971 |
| 390 mg SC Q3W | 1150 | 780 mg SC Q6W | 1380 | C1 | 0.833 |
| 390 mg SC Q3W | 1730 | 780 mg SC Q6W | 1780 | C2 | 0.972 |
| 395 mg SC Q3W | 1160 | 790 mg SC Q6W | 1400 | C1 | 0.829 |
| 395 mg SC Q3W | 1750 | 790 mg SC Q6W | 1800 | C2 | 0.972 |
| 410 mg SC Q3W | 1200 | 820 mg SC Q6W | 1450 | C1 | 0.828 |
| 410 mg SC Q3W | 1820 | 820 mg SC Q6W | 1870 | C2 | 0.973 |

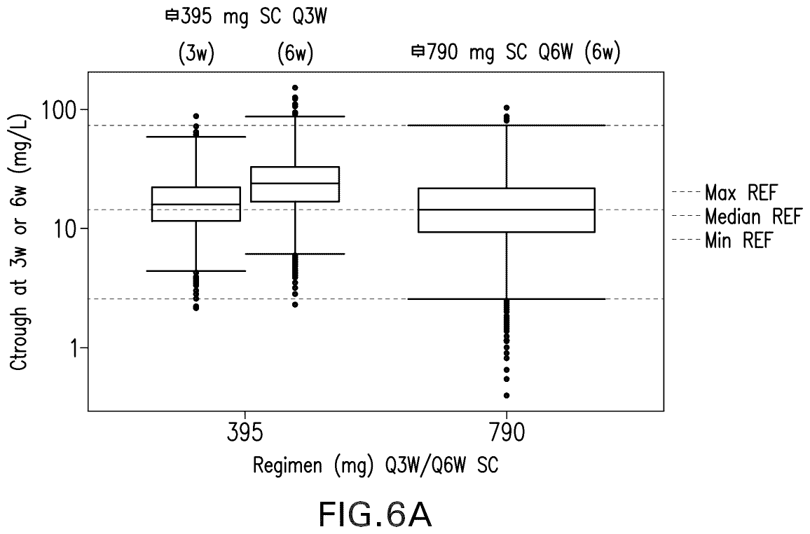

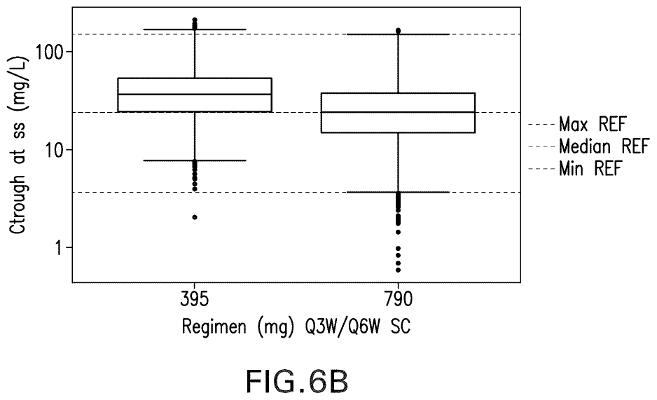

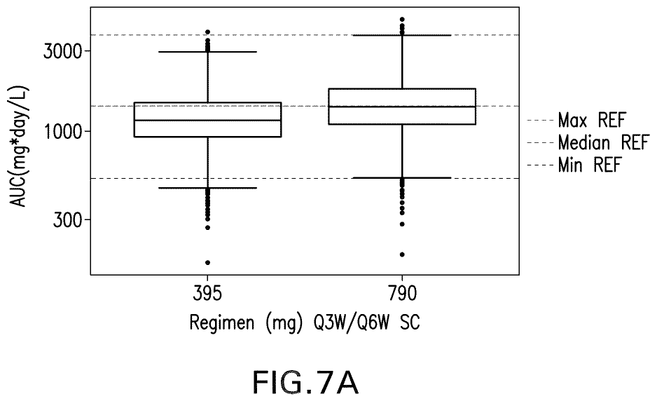

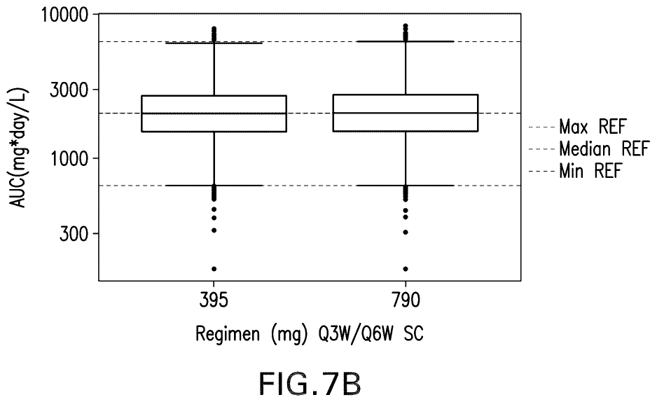

The selected dose of pembrolizumab SC with hyaluronidase PH20 variant 2 is 790 mg Q6W. The corresponding selected dose of pembrolizumab SC with hyaluronidase PH20 variant 2 is at Q3W is 395 mg. Based upon our analyses, efficacy is expected to be retained with SC pembrolizumab-HLN at the dose of 395 mg Q3W based on the following: C_{trough} at a 395 mg Q3W SC dose is expected to be ~15 to 60% higher than 790 mg Q6W SC, through treatment duration. Moreover, the distributions of C_{trough} largely overlap between SC-Q3W and SC-Q6W at both Cycle 1 and steady state. See FIGS. 6A and 6B. AUC_{0-6 wks} exposure at a 395 mg Q3W SC dose is expected to be similar to 790 mg Q6W SC, through treatment duration. Moreover, the distributions of AUC_{0-6 wks} largely overlap between SC-Q3W and SC-Q6W at both Cycle 1 and steady state. See FIGS. 7A and 7B. Safety is expected to be maintained with SC pembrolizumab-HLN at the dose of 395 mg Q3W given Cmax will be maintained lower than the 790 mg Q6W dose throughout treatment from cycle 1 through steady state.

FIGS. 6A-6B summarize the results of the population simulation including variability for C_{trough} for a dose of 395 mg Q3W SC and 790 mg Q6W SC of pembrolizumab-HLN. The simulations developed showed that the 395 mg Q3W SC dose leads to a range of C_{trough} across different patients that generally overlaps with the 790 mg Q6W SC dose. FIGS. 7A and 7B summarize the results of the population simulation including variability for AUC_{0-6 wks} for a dose of 395 mg Q3W SC and 790 mg Q6W SC of pembrolizumab-HLN. The simulations showed that the 395 mg Q3W SC dose leads to a range of AUC_{0-6 wks} across different patients that generally overlaps with the 790 mg Q6W SC dose. FIGS. 6A and 6B, 7A and 7B depict the distribution (5^{th}, 25^{th}, 50^{th}, 75^{th}, and 95^{th} percentiles) at cycle 1 and steady state of C_{trough} and AUC_{0-6 wks} respectively, using PK model-based simulations at a dose of 395 mg Q3W and 790 mg Q6W SC of pembrolizumab-HLN. Simulated PK exposure measures in 3105 subjects are shown.

Overall, the model-based simulations as supported by Tables 12A-D, FIGS. 6A, 6B, 7A and 7B indicate that a dose of 395 mg Q3W of pembrolizumab-HLN administered SC should lead to an optimal PK exposure profile that is consistent with that of 790 mg Q6W of pembrolizumab-HLN SC, thus maintaining efficacy, while also remaining within the clinical safety margin. In addition, based upon our analyses, 300-500 mg Q3W SC pembrolizumab-HLN doses are also expected to be safe and efficacious, and comparable to the PK exposure for 600-1000 mg Q6W SC pembrolizumab-HLN doses.

About 50 participants with unresectable, advanced melanoma will be allocated to Arm 4 of the phase I clinical trial in Table 7 (165 mg/mL pembrolizumab-HLN SC, 2.39 ml). Treatment cycles are 21 days (Q3W) at 395 mg pembrolizumab dose with pembrolizumab-HLN SC. PK exposure measures in Cycle 1 and at steady-state (Cycle 6), including Ctrough, Cmax, and AUC will be monitored. Overall survival, safety and anti-pembrolizumab antibodies will be monitored in Arm 4. Pembrolizumab trough concentration (Ctrough) after pembrolizumab-HLN SC treatment will be measured by collecting patient PK samples at predose (0-3 hours) on Day 1 of Cycles 1 and 6; any time on Days 2, 4, 6, 10, and 15 of Cycles 1 and 6. Pembrolizumab maximum plasma concentration (Cmax) after pembrolizumab-HLN SC treatment will be measured by collecting patient PK samples predose (0-3 hours) on Day 1 of Cycles 1 and 6; any time on Days 2, 4, 6, 10, and 15 of Cycles 1 and 6. Pembrolizumab area under the curve (AUC) after pembrolizumab-HLN SC treatment will be measured by collecting patient PK samples predose (0-3 hours) on Day 1 of Cycles 1 and 6; any time on Days 2, 4, 6, 10, and 15 of Cycles 1 and 6.

### Example 5

A six-weekly (Q6W) dosing schedule for SC pembrolizumab (without hyaluronidase) across multiple tumor types based on an evaluation using PK modeling and simulation with 400 mg Q6W IV as a reference regimen.

PK data from a phase 1 study of SC pembrolizumab-HLN as described above were generally consistent with PK data from the phase 1 study of SC pembrolizumab without hyaluronidase (KEYNOTE-555 Cohort A, same formulations in Table 6 without hyaluronidase). Results comparing both studies are shown in Table 13. As such, the range of bioavailability largely overlap for these formulations and data confirm that addition of hyaluronidase does not significantly impact PK of pembrolizumab SC administration. Thus, the distributions of PK exposures (C_{trough}, AUC, Cₘₐₓ) resulting from administration of a given dose of SC pembrolizumab with hyaluronidase or without hyaluronidase are expected to be similar. Hence, by inference the selected Q6W dose of 790 mg and dose range of 600 to 1000 mg identified for SC pembrolizumab with hyaluronidase are also expected to be safe and efficacious when applied to SC pembrolizumab without hyaluronidase.

TABLE 13
Pharmacokinetic data of subcutaneous formulation
of Pembrolizumab-HLN and Pembrolizumab

| | Pembrolizumab-HLN SC | Pembrolizumab SC |
|---|---|---|
| | (N = 81 subjects for PK | (N = 31 subjects for PK |
| | analysis) | analysis) |
| Bioavailability | 57% (range: | 66% (range: |
| | 38% to 75%) | 33% to 98%) |
| Tmax | 4 days (range: | 5.5 days (range: |
| | 2-35 days) | 3-14 days) |
| ADA | 1 of 83 | 0 of 36 |

### Example 6

### Pharmacokinetics of Pembrolizumab Across Tumor Types and Combination Therapy

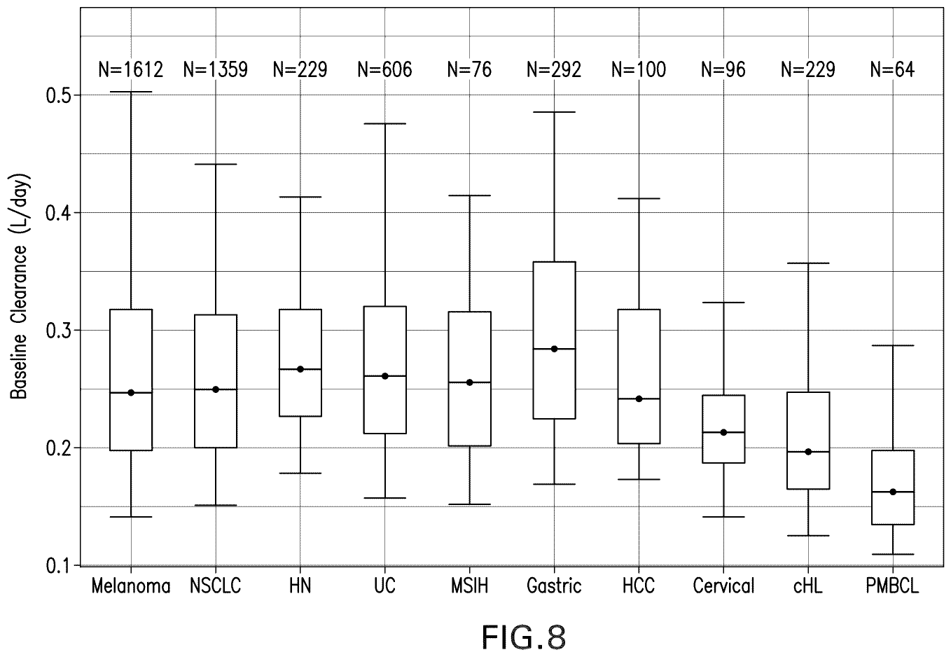

Over the course of pembrolizumab clinical development, extensive historical and emerging data from across multiple indications and treatment settings have indicated that both PK and immunogenicity are generally consistent across tumor types and between monotherapy and combination treatment. Furthermore, the exposure-response relationships of pembrolizumab for both efficacy and safety have been well established based on 8 randomized dose comparisons in melanoma and non-small lung cancer (NSCLC) and are shown to be flat in the clinically studied >5-fold dose/exposure range from 2 mg/kg Q3W to 10 mg/kg Q2W IV. A consistent, flat exposure-response relationship has also been observed for other indications (e.g., Head and Neck Squamous Cell Cancer (HNSCC), Classical Hodgkin Lymphoma (cHL), UC (urothelial cancer), GC (gastric cancer), PMBCL (Primary Mediastinal Large B-Cell Lymphoma), and MSI-H cancers) based on pooled analysis of data from treatment arms across studies (FIG. 8).

### Consistency of PK Across Tumor Types

The reference PK model of pembrolizumab, which serves as the basis for the pembrolizumab label, is based on conclusive analyses characterizing pembrolizumab PK based on a robust dataset of 2993 participants with melanoma or NSCLC from KEYNOTE-001, KEYNOTE-002, KEYNOTE-006, KEYNOTE-010, and KEYNOTE-024 (including doses of 2 mg/kg Q3W, 10 mg/kg Q3W, 10 mg/kg Q2W, and 200 mg Q3W). Tumor type did not have a meaningful impact on PK in the reference analysis. The model has been additionally evaluated for consistency of PK parameters across other approved indications (HNSCC, UC, GC, MSI-H cancers, cHL, PMBCL, HCC and cervical cancer). Data from participants with these cancer types were added to the reference dataset (based on melanoma and NSCLC), and the parameters of the reference model were re-estimated. This updated PK analysis including several approved tumor types yielded consistent model parameter estimates as the reference analysis based on melanoma and NSCLC.

Furthermore, the consistency of PK between individual tumor types and the reference PK model (melanoma and NSCLC) has been evaluated by overlaying observed concentrations against the 90% prediction interval of the model. These analyses revealed that observed concentrations decreased within the range of predicted concentrations, regardless of weight-based or fixed dosing; minor differences were seen in hematological tumors such as cHL and PMBCL that are not considered clinically meaningful. This indicates that the PK model was able to adequately describe pembrolizumab PK across tumor types, confirming the similarity in pembrolizumab PK across indications after IV administration.

### Consistency of PK Between Monotherapy and Combination Treatment

Chemotherapy is typically metabolized in the liver and does not typically have any effect on the disposition of monoclonal antibodies within the body. As is well described in the literature, monoclonal antibodies are predominantly catabolized by the human reticuloendothelial system. In general, PK interaction between pembrolizumab and small molecules is not pharmacologically expected, as metabolic or transporter pathways are not involved in the disposition of pembrolizumab.

In multiple clinical studies in which pembrolizumab was administered with chemotherapy (KEYNOTE-021, KEYNOTE-189: with carboplatin/cisplatin and pemetrexed; KEYNOTE-407: with carboplatin and paclitaxel/nab-paclitaxel; KEYNOTE-048: with carboplatin/cisplatin and 5-FU; and KEYNOTE-426: with axitinib), no effect was seen on overall exposure of pembrolizumab, as compared with that with pembrolizumab monotherapy.

### Applicability to Pembrolizumab-HLN Administration

In the context of SC administration, bioavailability and absorption are not expected to be impacted by tumor type or combination treatment (Anselmo A C et al., Nat Rev Drug Discov. 2019; 18:19-40) and given the distribution and elimination phases are same as IV, the consistency of PK across tumor types and treatment settings should be maintained. Therefore, pembrolizumab PK parameter comparisons made between SC and IV formulations based on the studies conducted in NSCLC in combination with chemotherapy should equally apply to draw inferences on matching exposures, and consequently on bridging efficacy and safety, between SC and IV for pembrolizumab indications.

1-67. (canceled)

68. A method of treating cancer in a human patient in need thereof comprising subcutaneously administering to the patient a dose of from 325 mg to 400 mg of an anti-PD-1 antibody, and a human hyaluronidase, every three weeks, wherein the anti-PD-1 antibody is a monoclonal antibody comprising: (a) a heavy chain comprising a sequence of amino acids as set forth in any one of SEQ ID NOs: 10-15, and (b) a light chain comprising a sequence of amino acids as set forth in SEQ ID NO:5.

69. The method of claim 68, wherein the dose of the anti-PD-1 antibody is 380 mg to 410 mg.

70. The method of claim 68, wherein the dose of the anti-PD-1 antibody is 395 mg.

71. The method of claim 68, wherein the anti-PD-1 antibody and the human hyaluronidase are co-formulated in a composition.

72. The method of claim 71, wherein about 700 Units to about 50000 Units of human hyaluronidase is co-formulated with the anti-PD-1 antibody.

73. The method of claim 72, wherein the composition comprises a dose of 395 mg of the anti-PD-1 antibody and 4800 Units of the human hyaluronidase.

74. The method of claim 68, wherein the human hyaluronidase consists of the amino acid sequence set forth in SEQ ID NO: 19.

75. The method of claim 68, wherein the human hyaluronidase consists of the amino acid sequence set forth in SEQ ID NO: 20.

76. The method of claim 70, wherein the human hyaluronidase consists of the amino acid sequence set forth in SEQ ID NO: 20.

77. The method of claim 68, wherein the cancer is a microsatellite instability-high (MSI-H) colorectal cancer or solid tumor, or the patient has a tumor with a high mutational burden which contains ≥10 mutations/megabase (Mut/Mb).

78. The method of claim 68, wherein the cancer is resected Stage IB, II, or IIIA non-small cell lung cancer.

79. The method of claim 68, wherein the cancer is metastatic non-small cell lung cancer wherein the patient has a tumor with PD-L1 expression as measured by a tumor proportion score (TPS) of ≥1%.

80. The method of claim 68, wherein the cancer is advanced or metastatic nonsquamous non-small cell lung cancer (NSCLC), and the patient's tumor has no EGFR or ALK genomic aberrations, and wherein the method further comprises administering a therapeutically effective amount of pemetrexed and platinum chemotherapy to the patient.

81. The method of claim 79, wherein the pemetrexed is administered by intravenous infusion to the patient in an amount of 500 mg/m² every 21 days, and the platinum chemotherapy is cisplatin administered to the patient in an amount of 75 mg/m² every 21 days.

82. The method of claim 68, wherein the cancer is squamous NSCLC and the method further comprises administering a therapeutically effective amount of carboplatin and paclitaxel or nab-paclitaxel.

83. The method of claim 82, wherein the carboplatin is administered by intravenous infusion at an AUC of 5-6 mg/ml/min, the paclitaxel is administered by intravenous infusion 200 mg/m² every 21 days, and the nab-paclitaxel is administered by intravenous infusion 100 mg/m² every 7 days.

84. The method of claim 68, wherein the cancer is metastatic, unresectable, or recurrent head and neck squamous cell cancer (HNSCC), and the patient's tumors express PD-L1 (CPS ≥1).

85. The method of claim 68, wherein the cancer is metastatic, unresectable, or recurrent head and neck squamous cell cancer (HNSCC), and the method further comprises administering platinum and 5-FU (Fluorouracil) to the patient.

86. The method of claim 68, wherein the cancer is relapsed or refractory classical Hodgkin lymphoma (cHL), refractory or relapsed primary mediastinal large B-cell lymphoma (PMBCL), unresectable or metastatic melanoma, or resected stage IIB, IIC, or III melanoma.

87. The method of claim 68, wherein the cancer is recurrent locally advanced unresectable or metastatic gastric cancer or gastroesophageal junction adenocarcinoma that is HER2-positive, the patient's tumor has a PD-L1 Combined Positive Score (CPS) ≥1, and the method further comprises administering trastuzumab, fluoropyrimidine and platinum-containing chemotherapy.

88. The method of claim 68, wherein the cancer is recurrent locally advanced or metastatic esophageal or gastroesophageal junction adenocarcinoma, and the method further comprises administering fluoropyrimidine-based chemotherapy and platinum-containing chemotherapy to the patient.

89. The method of claim 68, wherein the cancer is recurrent locally advanced or metastatic esophageal or gastroesophageal junction adenocarcinoma, wherein the patient's tumor has a PD-L1 Combined Positive Score (CPS) ≥10.

90. The method of claim 68, wherein the cancer is recurrent or metastatic cervical cancer, wherein the patient's tumor expresses PD-L1 (CPS≥1).

91. The method of claim 90, wherein the method further comprises administering paclitaxel and cisplatin or paclitaxel and carboplatin, with or without bevacizumab.

92. The method of claim 68, wherein the cancer is cutaneous squamous cell carcinoma.

93. The method of claim 68, wherein the cancer is advanced or metastatic renal cell carcinoma (RCC).

94. The method of claim 68, wherein the cancer is RCC at intermediate-high or high risk of recurrence following nephrectomy, or following nephrectomy and resection of metastatic lesions, and the method comprises administering the anti-PD-1 antibody for adjuvant treatment.

95. The method of claim 68, wherein the cancer is advanced endometrial carcinoma that is not MSI-H or mismatch repair deficient (dMMR).

96. The method of claim 68, wherein the cancer is recurrent, unresectable or metastatic triple negative breast cancer (TNBC), and the patient's tumor expresses PD-L1 as measured by a Combined Positive Score (CPS) ≥10, and the method further comprises administering to the patient carboplatin and paclitaxel.

97. The method of claim 68, wherein the cancer is high-risk early stage or locally advanced TNBC, and the method comprises administering the anti-PD-1 antibody and carboplatin and paclitaxel for neoadjuvant treatment, and then administering the anti-PD-1 antibody, doxorubicin and cyclophosphamide.


SEQUENCE LISTING (25 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 446 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 447 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 446 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 445 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 15 - protein, 445 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 16 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 17 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 18 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 19 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 20 - protein, 447 aa, synthetic construct [sequence omitted]

SEQ ID NO: 21 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 22 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELK

SEQ ID NO: 23 - protein, 122 aa, synthetic construct
QVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 24 - protein, 112 aa, synthetic construct
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 25 - protein, 122 aa, synthetic construct
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS